(19) United States
(12) Patent Application Publication
(10) Pub. No.: US 2019/0177735 A1
(43) Pub. Date: Jun. 13, 2019

(54) METHODS AND COMPOSITIONS FOR MODIFICATION OF PLASTID GENOMES

(71) Applicant: North Carolina State University, Raleigh, NC (US)

(72) Inventors: Heike I. Sederoff, Raleigh, NC (US); Chase Lawrence Beisel, Raleigh, NC (US); Colin Murphree, Raleigh, NC (US); Soundarya Srirangan, Raleigh, NC (US)

(21) Appl. No.: 16/327,505

(22) PCT Filed: Sep. 1, 2017
(86) PCT No.: PCT/US2017/049913

§ 371 Date: Feb. 22, 2019

Related U.S. Application Data
(60) Provisional application No. 62/383,074, filed on Sep. 2, 2016.

(51) Int. Cl. C12N 15/82

(52) U.S. Cl. CPC C12N 15/8214; C12N 2310/20; C12N 15/8213

(57) ABSTRACT

The invention relates to methods of modifying a plastid genome using sequence specific nucleases, as well as reverse transcriptase polypeptides and plastid modification cassettes. The invention further relates to methods of modifying a plastid or a mitochondrial genome using ATP-dependent DNA ligase D (LigD) and DNA-binding protein Ku (Ku). Also included are the plants, plant cells, and seeds produced by these methods.

### PRIORITY

This application claims priority from U.S. Provisional Application No. 62/383,074, filed on Sep. 2, 2016, the contents of which are incorporated herein by reference in their entireties.

### STATEMENT OF GOVERNMENT SUPPORT

This invention was made with government support under grant number DE-AR0000207 awarded by the United States Department of Energy (DOE) and grant number 1639429 awarded by the National Science Foundation (NSF). The government has certain rights to this invention.

### FIELD

The invention relates to methods of modifying a plastid genome using sequence specific nucleases, as well as reverse transcriptase polypeptides and plastid modification cassettes. The invention further relates to methods of modifying a plastid genome and/or a mitochondrial genome using ATP-dependent DNA ligase D (LigD) and DNA-binding protein Ku (Ku). Also included are the plants, plant cells, and seeds produced by these methods.

### BACKGROUND

Plant plastids are an excellent compartment for the expression, of transgenes because they can produce high levels of protein (Daniell et al., 2009; Oey et al., 2009a; Ruhlman et al., 2010; Ruf et al. 2001). It is also significant that in the majority of flowering plants including major crops, inheritance of the plastid genome is through the maternal parent (Corriveau and Coleman, 1988), and transmission of plastids through pollen is rare (Ruf et al., 2007; Svab and Maliga, 2007). Thus, plastid transformation provides a strong level of biological containment. Another advantage is that the integration of a transgene in the plastid genome proceeds by homologous recombination and is therefore precise and predictable. Hence, variable position effects on gene expression or the inadvertent inactivation of a host gene by integration of the transgene is avoided. Furthermore, plastid genes are not subject to gene silencing or RNA interference. It is also noteworthy that multiple transgenes organized as a polycistronic unit can be expressed from the plastid genome (Staub and Maliga, 1995; Quesada-Vargas et al., 2005). For general current reviews of plastid transformation see Bock (2015) or Maliga (2012).

Industrial and pharmaceutical proteins have successfully been produced in plant chloroplasts (“molecular farming” or “pharming”) and several companies have formed to utilize the advantages of producing pharmaceutical enzymes and vaccines in plants (for review: Sack et al. 2015, Stoger et al. 2014). Product-specific benefits of plant-based systems have also been exploited, including bioencapsulation and the mucosal delivery of minimally processed topical and oral products with a lower entry barrier than pharmaceuticals for injection (Sack et al. 2015; Stoger et al. 2014). A new and interesting use of plastid transformation is the expression of double-stranded RNAs in plastids to confer insect resistance to crops (Zhang et al. 2015) For review of herbicide and insect resistance through chloroplast transformation see Bock (2007).

However, genetic engineering of the prokaryote-like genome in plastids has been successful only in a few plant species using a biolistic approach (Boynton et al. 1988; Svab et al. 1990), or PEG mediated introduction of transgenes (Golds et al., 1993). Chloroplasts of important crops including rice, wheat and maize have not yet been reproducibly or efficiently stably transformed and/or cannot be made homoplasmic (Bock 2015). Attempts to stably transform the chloroplast of the model plant Arabidopsis thaliana so far have been unsuccessful.

Although the plastid genome is relatively small in size (ca. 120-240kb), chloroplasts are highly polyploid and carry between 80 (e.g., Chlamydomonas) and thousands of copies per chloroplast. Most flowering plants have many chloroplasts per cell (10-100). Therefore, to generate a transgenic homoplasmic (all plastid chromosomes in a plant are identical and not segregating) plant, usually several segregating generations must be screened and selected, or several iterations of tissue culture regeneration are required, making the process lengthy and expensive. In some crop plants (e.g. rice), homoplasmy has not been achieved

### SUMMARY

Shortcomings of the plastid modification and transformation systems developed thus far are addressed by the present specification through methods that will enable precision editing and modification of the plastid genome that can result in homoplasmic transplastomic plants useful, for example, for producing commercial products and for research.

One aspect of the invention provides a method of modifying a plastid genome of a plant cell, comprising: introducing into a plant cell (a) a polynucleotide encoding a reverse transcriptase (RT) polypeptide fused to a plastid transit peptide; (b) a recombinant nucleic acid comprising (i) a plastid modification cassette, (ii) a first recognition site located immediately 5′ of the plastid modification cassette, (iii) a second recognition site located immediately 3′ of the plastid modification cassette, and (iv) a plastid localization sequence operably located 5′ of the first recognition site; and (c) a polynucleotide encoding a sequence-specific nuclease fused to a plastid transit peptide, thereby modifying the plastid genome of said plant cell.

A second aspect provides a modifying a plastid genome of a plant cell, comprising introducing into a plant cell a polynucleotide encoding an ATP-dependent DNA ligase D (LigD) fused to a plastid transit peptide and a polynucleotide encoding a DNA-binding protein Ku (Ku) fused to a plastid transit peptide, thereby modifying the plastid genome of said plant cell.

A third aspect provides a method of modifying a plastid genome of a plant cell, comprising introducing into a plant cell (a) a recombinant nucleic acid linked to a plastid localization sequence and comprising a polynucleotide encoding a reverse transcriptase polypeptide and a sequence-specific nuclease; and (b) a recombinant nucleic acid comprising (i) a plastid modification cassette, (ii) a first recognition site located immediately 5′ of the plastid modification cassette, (iii) a second recognition site located immediately 3′ of the plastid modification cassette, and (iv) a plastid localizing sequence operably located 5′ of the first recognition site, thereby modifying the plastid genome of the plant cell.

A fourth aspect provides method of producing a plant cell having a modified plastid genome, comprising introducing into a plant cell (a) a polynucleotide encoding a reverse transcriptase (RT) polypeptide fused to a plastid transit peptide; (b) a recombinant nucleic acid comprising (i) a plastid modification cassette, (ii) a first recognition site located immediately 5′ of the plastid modification cassette, (iii) a second recognition site located immediately 3′ of the plastid modification cassette, and (iv) a plastid localization sequence operably located 5′ of the first recognition site; and (c) a polynucleotide encoding a sequence-specific nuclease fused to a plastid transit peptide, thereby producing a plant cell having a modified plastid genome.

A fifth aspect provides a method of producing a plant cell having a modified plastid genome, comprising introducing into a plant cell (a) a recombinant nucleic acid linked to a plastid localization sequence and comprising a polynucleotide encoding a reverse transcriptase polypeptide and a sequence-specific nuclease; and (b) a recombinant nucleic acid comprising (i) a plastid modification cassette, (ii) a first recognition site located immediately 5′ of the plastid modification cassette, (iii) a second recognition site located immediately 3′ of the plastid modification cassette, and (iv) a plastid localizing sequence operably located 5′ of the first recognition site, thereby modifying the plastid genome of the plant cell.

A sixth aspect provides a method of expressing a polynucleotide sequence of interest (POI) in a plastid, comprising introducing into a plant cell: (a) a polynucleotide encoding a reverse transcriptase polypeptide fused to a plastid transit peptide and a sequence-specific nuclease fused to a plastid transit peptide, or a polynucleotide linked to a plastid localization sequence and encoding a reverse transcriptase polypeptide and a sequence-specific nuclease; and (b) a recombinant nucleic acid comprising (i) a plastid modification cassette, (ii) a first recognition site located immediately 5′ of the plastid modification cassette, (iii) a second recognition site located immediately 3′ of the plastid modification cassette, and (iv) a plastid localization sequence operably located 5′ of the first recognition site, wherein said plastid modification cassette comprises a POI, thereby expressing the POI in a plastid

A seventh aspect provides a method of transforming a plastid genome, comprising: introducing into a plant cell: (a) a polynucleotide encoding a reverse transcriptase polypeptide fused to a plastid transit peptide and a sequence-specific nuclease fused to a plastid transit peptide, or a polynucleotide linked to a plastid localization sequence and encoding a reverse transcriptase polypeptide and a sequence-specific nuclease; and (b) a recombinant nucleic acid comprising (i) a plastid modification cassette, (ii) a first recognition site located immediately 5′ of the plastid modification cassette, (iii) a second recognition site located immediately 3′ of the plastid modification cassette, and (iv) a plastid localization sequence operably located 5′ of the first recognition site, wherein said plastid modification cassette comprises a POI, thereby transforming said plastid genome.

Additionally provided are the plants, plant parts, and plant cells comprising a plastid genome modified using the method of the invention as well as crops and products produced therefrom. In some particular aspects, the invention provides seeds and progeny plants produced from the plants of the invention.

The foregoing and other aspects of the present invention will now be described in more detail with respect to other embodiments described herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art. Therefore, aspects of the invention that are described with respect to one embodiment may be incorporated in a different embodiment although not specifically described relative thereto. That is, all embodiments and/or features of any embodiment can be combined in any way and/or combination. Applicant reserves the right to change any originally filed claim and/or file any new claim accordingly, including the right to be able to amend any originally filed claim to depend from and/or incorporate any feature of any other claim or claims although not originally claimed in that manner. These and other objects and/or aspects of the present invention are explained in detail in the specification set forth below. Further features, advantages and details of the present invention will be appreciated by those of ordinary skill in the art from a reading of the figures and the detailed description of the preferred embodiments that follow, such description being merely illustrative of the present invention.

### BRIEF DESCRIPTION OF THE DRAWINGS

FIGS. 1A-1C show a schematic of exemplary transformation constructs and vector. Construct element abbreviations and symbols used: FIG. 1A: NUC_{pro/ter} (generic nuclear promoter and terminator used for gene expression); Chp TP, (plastid transit peptide). hCas9 (human codon optimized CRISPR associated protein 9); ovals, 6×His tag; FIG. 1B: PC-GW-mCherry vector used for cloning and expression of the Cas9 cassette (FIG. 1A.); FIG. 1C: attR1 and attR2 recombination sites for sub cloning into PC-GW series vectors (e.g. PC-GW-mCherry); AtU6 ₚᵣₒ (Arabidopsis thaliana U6 RNA polymerase III promoter); ELVd (Eggplant Latent Viroid derived non-coding RNA sequence); gRNA (guide RNA).

FIG. 2 shows the targeted guide RNA's designed for the precise editing of Ath psbH and Ath psbA gene models with the CRISPR/Cas technology. Dark grey shading and light grey shading in the sequence represents gRNA and protospacer adjacent motif region, respectively.

FIGS. 3A-3C show a schematic of the exemplary constructs for use in plastid transformation. Construct element abbreviations and symbols used: FIG. 3A: NUC_{pro/ter} (generic nuclear promoter and terminator used for gene expression); Chp TP, (plastid transit peptide). RT (Reverse transcriptase); ovals, 3XFLAG tag; FIG. 3B: FRS (First Recognition Sequence Comprising the R, V 5′ UTR, and PBS sequences), R (Repeat containing sequences used during reverse transcription for strong stop DNA transfer, the two R sequences are identical), V 5′ UTR (untranslated region found between R and PBS sequences (Primer Binding Sequences used for mRNA attachment to tRNA primer)), a plastid modification cassette, SRS (Second Recognition Sequence comprising PPT, V 3′ UTR, and R sequences), PPT (polypurine tag used by reverse transcriptase to initiate second strand DNA synthesis after RNAseH cleavage), V 3′ UTR (untranslated region between PPT and R sequences); FIG. 3C: LTS/RTS (homology arms used for homologous recombination with plastid genome), Chp_{pro/ter} (generic chloroplast promoter and terminator), 5′ UTR/3′ UTR (optional untranslated regions). GOI, any gene/polynucleotide of interest.

FIG. 4 shows an example transformation cassette for the nuclear transformation of plastid-targeted ATP-dependent DNA ligase D (LigD) and DNA-binding protein Ku (Ku) to assist DNA repair mechanisms in plastids.

FIG. 5 shows an example expression cassette carrying a gene coding for the Murine Moloney Reverse Transcriptase.

FIG. 6 shows the heritability of the gene coding for the reverse transcriptase protein in plants. DNA was extracted from plants suspected to carry the reverse transcriptase gene, and that gene was identified using Polymerase Chain Reaction (PCR). The presence of a band indicates the presence of the RT gene. Lanes labeled “C” contain DNA from wild (nontransgenic) Arabidopsis thaliana, none of which produce a band. The other lanes are extracted from transgenic plants that are in the first generation (T1), or progeny of the first generation (second generation, or T2).

FIG. 7 is a western blot that provides a comparison between extraction techniques in recovering reverse transcriptase produced by tobacco. Lane 1 is protein extracted from normal tobacco, whereas lanes 2, 3, and 4 are protein extracted from transgenic tobacco expressing chloroplast targeted reverse transcriptase. Lanes 2, 3, and 4 differ in extraction methods, the major difference being that protein in lanes 2 and 4 are extracted with high levels of detergent, whereas lane 3 is essentially water and salt.

FIG. 8 is a western blot that shows that RT protein can be produced in stable lines (heritable) of Arabidopsis thaliana.

FIG. 9 shows an example plastid modification cassette. The cassette was synthesized and cloned at HindIII site in pUC57 vector by Genscript. Cassettes were sub-cloned into PC-GW-mCherry (plant transformation vector) (Genbank accession: KP826771) by gateway cloning.

FIG. 10 shows the exemple construct of FIG. 9 in more detail.

FIG. 11 shows an example of a workflow design for generating transgenic Arabidopsis and confirming the presence of the transgene introduced in a plastid modification cassette. The plastid modification cassette is inserted into Arabidopsis cells via agrobacterium using the floral dip method. Here, the plastid modification cassette construct co-expresses a fluorescent protein (marker gene) that allows us to identify plants containing the construct by segregating seed. Plants were verified to be transgenic by PCR analysis.

FIG. 12A-12C show that the transgenes in the plastid modification cassette are heritable and expressed. FIG. 12A shows identification of plants carrying the plastid modification cassette through the use of mcherry fluorescence sorting (the seeds fluoresce red). FIG. 12B shows about 80 plants identified from mcherry fluorescence sorting growing in a greenhouse. FIG. 12C shows Arabidopsis thaliana lines which carry the full length plastid modification cassette. The plants which carry the cassette are revealed by the presence of a single dark band.

FIGS. 13A-13C show that plastid modification cassette RNA is expressed and the expression is heritable. FIGS. 13A-13B provide the results of quantitative PCR assays showing the abundance of RNA in plastid modification cassette expressing tobacco and wild type tobacco. FIG. 13C shows both tobacco and Arabidopsis lines express and maintain the plastid modification cassette.

FIG. 14 shows an example process for converting a recombinant eukaryotic Cas9 (targeted to the nucleus) into one targeted to the chloroplast via the replacement of nuclear localization signal (NLS) with a chloroplast transit peptide transit peptide (TP) sequence. 3×FLAG: Flag epitope tag commonly used to identify and purify recombinant proteins in plants. 6× His: Hexahistidine tag commonly used to identify and purify recombinant proteins.

FIG. 15 shows second generation Arabidopsis plants that carry a chloroplast targeted Cas9 gene.

FIG. 16A-16B are western blots showing the production of chloroplast targeted Cas9 protein in transgenic Arabidopsis (FIG. 16A) and Tobacco (FIG. 16B). . The “+ ctrl” lane in is a commercially prepared Cas9 protein produced using E. coli. “Col 0” represents wild type Arabidopsis material. Two first generation transgenic Arabidopsis plants are shown to produce the chloroplast localized Cas9 protein (FIG. 16A). The single dark band in lane 2 of FIG. 16B represents Cas9 protein produced in transgenic Tobacco.

FIG. 17 shows that sgRNA cassettes comprise the correct cassette sequence. Each of the 10 sgRNA cassettes have a unique 20 nucleotide target site that is amplified via PCR.

FIG. 18 shows transient sgRNA in tobacco.

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measureable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein,, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, “chimeric” refers to a nucleic acid molecule or a polypeptide in which at least two components are derived from different sources (e.g., different organisms, different coding regions).

“Complement” as used herein can mean 100% complementarity or identity with the comparator nucleotide sequence or it can mean less than 100% complementarity (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like, complementarity).

The terms “complementary” or “complementarity,” as used herein, refer to the natural binding of polynucleotides under permissive salt and temperature conditions by base-pairing. For example, the sequence “A-G-T” binds to the complementary sequence “T-C-A” (5′ to3′). Complementarity between two single-stranded molecules may be “partial,” in which only some of the nucleotides bind, or it may be complete when total complementarity exists between the single stranded molecules. The degree of complementarity between nucleic acid strands has significant effects on the efficiency and strength of hybridization between nucleic acid strands.

A “fragment” or “portion” of a nucleotide sequence of the invention will be understood to mean a nucleotide sequence of reduced length relative (e.g., reduced by 1, 2, 3, 4, 5, 6, 7, 8, 9, 10, 11, 12, 13, 14, 15, 16, 17, 18, 19, 20 or more nucleotides) to a reference nucleic acid or nucleotide sequence and comprising, consisting essentially of and/or consisting of a nucleotide sequence of contiguous nucleotides identical or substantially identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to the reference nucleic acid or nucleotide sequence. Such a nucleic acid fragment or portion according to the invention may be, where appropriate, included in a larger polynucleotide of which it is a constituent. In some embodiments, a repeat of a spacer-repeat sequence can comprise a fragment of a repeat sequence of a wild-type CRISPR locus or a fragment of repeat sequence of a synthetic CRISPR array, wherein the fragment of the repeat retains the function of a repeat in a CRISPR array of hybridizing with the tracr nucleic acid and being bound by the Cas9 polypeptide or wherein the fragment of the repeat retains the function of a repeat in a CRISPR array of hybridizing necessary for being functional with a Cpf1 polypeptide. In some embodiments, the invention may comprise a functional fragment of a Cas9 nuclease or a Cpf1 nuclease. A Cas9 or Cpf1 functional fragment retains one or more of the activities of a native Cas9 or Cpf1 nucleases including, but not limited to, nuclease activity (e.g., HNH nuclease activity, RuvC or RuvC-like nuclease activity), DNA, RNA and/or PAM recognition and binding activities. A functional fragment of a Cas9 nuclease may be encoded by a fragment of a Cas9 polynucleotide. A functional fragment of a Cpf1 nuclease may be encoded by a fragment of a Cpf1 polynucleotide.

As used herein, the term “gene” refers to a nucleic acid molecule capable of being used to produce sgRNA, mRNA, antisense RNA, RNAi (miRNA, siRNA, shRNA), anti-microRNA antisense oligodeoxyribonucleotide (AMO), and the like. Genes may or may not be capable of being used to produce a functional protein or gene product. Genes can include both coding and non-coding regions (e.g., introns, regulatory elements, promoters, enhancers, termination sequences and/or 5′ and 3′ untranslated regions). A gene may be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

A “heterologous” or a “recombinant” nucleic acid is a nucleotide sequence not naturally associated with a host cell into which it is introduced, including non-naturally occurring multiple copies of a naturally occurring nucleotide sequence or a promoter operably linked to a nucleic acid sequence to which it is not naturally operably linked.

As used herein, “modify,” “modifying” or “modification” (and grammatical variations thereof) of a plastid genome means any alteration of the genome of a plastid. Such modifications can include, but are not limited to, deleting one or more nucleotides or an entire nucleic acid region (transcribed and untranscribed regions), altering (inhibiting and/or activating) the expression of an endogenous polynucleotide, introducing one or more point mutations, introducing a synthetic promoter, introducing a regulatory RNA, introducing a polynucleotide to be expressed from an endogenous operon (e.g., no promoter or other regulatory sequence introduced), introducing a gene expression construct; and/or introducing an operon expression construct, and the like. Thus, in some embodiments, modifying a plastid genome comprises transforming a plastid genome. These and other modifications of the plastid genome can be carried out using the constructs and methods described herein.

Different nucleic acids or proteins having homology are referred to herein as “homologues.” The term homologue includes homologous sequences from the same and other species and orthologous sequences from the same and other species. “Homology” refers to the level of similarity between two or more nucleic acid and/or amino acid sequences in terms of percent of positional identity (i.e., sequence similarity or identity). Homology also refers to the concept of similar functional properties among different nucleic acids or proteins. Thus, the compositions and methods of the invention further comprise homologues to the nucleotide sequences and polypeptide sequences of this invention. “Orthologous,” as used herein, refers to homologous nucleotide sequences and/ or amino acid sequences indifferent species that arose from a common ancestral gene during speciation. A homologue of a nucleotide sequence of this invention has a substantial sequence identity (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and/or 100%) to said nucleotide sequence of the invention. Thus, for example, a homologue of any sequence-specific nuclease (e.g., Cpf1, Cas9, meganuclease, TALEN, ZFN), reverse transcriptase, Ku or LigD useful with this invention may be at least about 70% homologous or more to any other sequence-specific sequence, reverse transcriptase, Ku or LigD, respectively.

As used herein, hybridization, hybridize, hybridizing, and grammatical variations thereof, refer to the binding of two fully complementary nucleotide sequences or substantially complementary sequences in which some mismatched base pairs may be present. The conditions for hybridization are well known in the art and vary based on the length of the nucleotide sequences and the degree of complementarity between the nucleotide sequences. In some embodiments, the conditions of hybridization can be high stringency, or they can be medium stringency or low stringency depending on the amount of complementarity and the length of the sequences to be hybridized. The conditions that constitute low, medium and high stringency for purposes of hybridization between nucleotide sequences are well known in the art (See, e.g., Gasiunas et al. (2012) Proc. Natl. Acad. Sci. 109:E2579-E2586; M. R. Green and J. Sambrook (2012) Molecular Cloning: A Laboratory Manual. 4th Ed., Cold Spring Harbor Laboratory Press, Cold Spring Harbor, N.Y.).

As used herein, the terms “increase,” “increasing,” “increased,” “enhance,” “enhanced,” “enhancing,” and “enhancement” (and grammatical variations thereof) describe an elevation of at least about 15%, 25%, 50%, 75%, 100%, 150%, 200%, 300%, 400%, 500% or more as compared to a control. Thus, for example, increased transcription of a target DNA can mean an increase in the transcription of the target gene of at least about 15%, 25%, 50%, 75%, 100%, 150%, 200%, 300%, 400%, 500% or more as compared to a control. Thus, for example, the increase in expression of a polypeptide can mean an increase of about 15%, 25%, 50%, 75%, 100%, 150%, 200%, 300%, 400%, 500% or more as compared expression of said polypeptide in a control plant or plant cell that has not been modified according to this invention.

As used herein, the terms “reduce,” “reduced,” “reducing,” “reduction,” “diminish,” “suppress,” and “decrease” (and grammatical variations thereof), describe, for example, a decrease of at least about 5%, 10%, 15%, 20%, 25%, 35%, 50%, 75%, 80%, 85%, 90%, 95%, 97%, 98%, 99%, or 100% as compared to a control. In particular embodiments, the reduction can result in no or essentially no (i.e., an insignificant amount, e.g., less than about 10% or even 5%) detectable activity or amount. Thus, for example, reduced transcription of a target DNA can mean a reduction in the transcription of the target gene of at least about 5%, 10%, 15%, 20%, 25%, 35%, 50%, 75%, 80%, 85%, 90%, 95%, 97%, 98%, 99%, or 100% as compared to a control.

A “native” or “wild type” nucleic acid, nucleotide sequence, polypeptide or amino acid sequence refers to a naturally occurring or endogenous nucleic acid, nucleotide sequence, polypeptide or amino acid sequence. Thus, for example, a “wild type mRNA” is an mRNA that is naturally occurring in or endogenous to the organism. A “homologous” nucleic acid sequence is a nucleotide sequence naturally associated with a host cell into which it is introduced.

Also as used herein, the terms “nucleic acid,” “nucleic acid molecule,” “nucleic acid construct,” “nucleotide sequence” and “polynucleotide” refer to RNA or DNA that is linear or branched, single or double stranded, or a hybrid thereof. The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made. The nucleic acid constructs of the present disclosure can be DNA or RNA, but are preferably DNA. Thus, although the nucleic acid constructs of this invention may be described and used in the form of DNA, depending on the intended use, they may also be described and used in the form of RNA.

As used herein, the term “nucleotide sequence” refers to a heteropolymer of nucleotides or the sequence of these nucleotides from the 5′ to 3′ end of a nucleic acid molecule and includes DNA or RNA molecules, including cDNA, a DNA fragment or portion, genomic DNA, synthetic (e.g., chemically synthesized) DNA, plasmid DNA, mRNA, and anti-sense RNA, any of which can be single stranded or double stranded. The terms “nucleotide sequence” “nucleic acid,” “nucleic acid molecule,” “oligonucleotide” and “polynucleotide” are also used interchangeably herein to refer to a heteropolymer of nucleotides. All nucleic acids provided herein have 5′ and 3′ ends. Further, except as otherwise indicated, nucleic acid molecules and/or nucleotide sequences provided herein are presented herein in the 5′ to 3′ direction, from left to right and are represented using the standard code for representing the nucleotide characters as set forth in the U.S. sequence rules, 37 CFR §§ 1.821-1.825 and the World Intellectual Property Organization (WIPO) Standard ST.25.

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence.

Any plant can be employed in practicing this invention including an angiosperm, a gymnosperm, a monocot, a dicot, a C3, C4, and/or CAM plant, and/or an algae (e.g., a microalgae, and/or a macroalgae).

The term “plant part,” as used herein, includes but is not limited to reproductive tissues (e.g., petals, sepals, stamens, pistils, receptacles, anthers, pollen, flowers, fruits, flower bud, ovules, seeds, embryos, nuts, kernels, ears, cobs and husks); vegetative tissues (e.g., petioles, stems, roots, root hairs, root tips, pith, coleoptiles, stalks, shoots, branches, bark, apical meristem, axillary bud, cotyledon, hypocotyls, and leaves); vascular tissues (e.g., phloem and xylem); specialized cells such as epidermal cells, parenchyma cells, chollenchyma cells, schlerenchyma cells, stomates, guard cells, cuticle, mesophyll cells; callus tissue; and cuttings. The term “plant part” also includes plant cells, including plant cells that are intact in plants and/or parts of plants, plant protoplasts, plant tissues, plant organs, plant cell tissue cultures, plant calli, plant clumps, and the like. As used herein, “shoot” refers to the above ground parts including the leaves and stems. As used herein, the term “tissue culture” encompasses cultures of tissue, cells, protoplasts and callus.

As used herein, “plant cell” refers to a structural and physiological unit of the plant, which typically comprise a cell wall but also includes protoplasts. A plant cell of the present invention can be in the form of an isolated single cell or can be a cultured cell or can be a part of a higher-organized unit such as, for example, a plant tissue (including callus) or a plant organ. In some embodiments, a plant cell can be an algal cell.

Non-limiting examples of a plant or part thereof of the present invention include woody, herbaceous, horticultural, agricultural, forestry, nursery, ornamental plant species and plant species useful in the production of biofuels, and combinations thereof.

In some embodiments of this invention, a plant, plant part or plant cell can be from a genus including, but not limited to, the genus of Arabidopsis, Zea, Nicotiana, Solanum, Triticum, Musa, Camelina, Sorghum, Gossypium, Brassica, Allium, Armoracia, Poa, Agrostis, Lolium, Festuca, Calamogrostis, Deschampsia, Spinacia, Beta, Pisum, Chenopodium, Helianthus, Pastinaca, Daucus, Petroselium, Populus, Prunus, Castanea, Eucalyptus, Acer, Quercus, Salix, Juglans, Picea, Pinus, Abies, Lemna, Wolffia, Spirodela, Oryza or Gossypium.

In some embodiments, a plant, plant part or plant cell can be from a species including, but not limited to, the species of Camelina alyssum (Mill.) Thell., Camelina macrocarpa Andrz. ex DC., Camelina rumelica Velen., Camelina sativa (L.) Crantz, Sorghum bicolor (e.g.,

Sorghum bicolor L. Moench), Gossypium hirsutum, Brassica oleracea, Brassica rapa, Brassica napus, Raphanus sativus, Armoracia rusticana, Allium sative, Allium cepa, Populus grandidentata, Populus tremula, Populus tremuloides, Prunus serotina, Prunus pensylvanica, Castanea dentate, Populus balsamifer, Populus deltoids, Acer Saccharum, Acer nigrum, Acer negundo, Acer rubrum, Acer saccharinum, Acer pseudoplatanus or Oryza sativa. In some embodiments, the plant, plant part or plant cell can be, but is not limited to, a plant of, or a plant part, or plant cell from turfgrass (bluegrass, bentgrass, ryegrass, fescue), feather reed grass, tufted hair grass, spinach, beets, chard, quinoa, sugar beets, lettuce, sunflower (Helianthus annuus), peas (Pisum sativum), parsnips (Pastinaca sativa), carrots (Daucus carota), parsley (Petroselinum crispum), duckweed, apple, tomato, pear, pepper (Capsicum), bean (e.g., green and dried), cucurbits (e.g., squash, cucumber, honeydew melon, watermelon, cantaloupe, and the like), papaya, mango, pineapple, avocado, stone fruits (e.g., plum, cherry, peach, apricot, nectarine, and the like), grape (wine and table), strawberry, raspberry, blueberry, mango, cranberry, gooseberry, banana, fig, citrus (e.g., clementine, kumquat, orange, grapefruit, tangerine, mandarin, lemon, lime, and the like), nuts (e.g., hazelnut, pistachio, walnut, macadamia, almond, pecan, and the like), lychee (Litchi), soybean, corn, sugar cane, peanuts, cotton, canola, oilseed rape, sunflower, rapeseed, alfalfa, timothy, tobacco, tomato, sugarbeet, potato, sweetpotato, pea, carrot, cereals (e.g., wheat, rice, barley, rye, millet, sorghum, oat, triticale, and the like), buckwheat, roses, tulips, violets, basil, oil palm, elm, ash, oak, maple, fir, spruce, cedar, pine, birch, cypress, eucalyptus, willow, coffee, miscanthus, arundo, and/or switchgrass.

In further embodiments, a plant and/or plant cell can be an alga or alga cell from a class including, but not limited to, the class of Bacillariophyceae (diatoms), Haptophyceae, Phaeophyceae (brown algae), Rhodophyceae (red algae) or Glaucophyceae (red algae). In still other embodiments, a plant and/or plant cell can be an algae or algae cell from a genus including, but not limited to, the genus of Achnanthidium, Actinella, Nitzschia, Nupela, Geissleria, Gomphonema, Planothidium, Halamphora, Psammothidium, Navicula, Eunotia, Stauroneis, Chlamydomonas, Dunaliella, Nannochloris, Nannochloropsis, Scenedesmus, Chlorella, Cyclotella, Amphora, Thalassiosira , Phaeodactylum, Chrysochromulina, Prymnesium, Thalassiosira, Phaeodactylum, Glaucocystis, Cyanophora, Galdieria, or Porphyridium. Additional nonlimiting examples of genera and species of diatoms useful with this invention are provided by the US Geological Survey/Institute of Arctic and Alpine Research at westerndiatoms.colorado.edu/species.

As used herein, a “plastid” refers to a group of double membrane organelles found in plants, which vary in structure and function and contain DNA. A plastid can include, but is not limited to, a chloroplast, a leucoplast, an amyloplast, an etioplast, a chromoplast, a rhodoplast, a muroplast, an elaioplast, a proteinoplast and/or a proplastid.

Any reverse transcriptase (RT) polypeptide may be used with this invention. Exemplary RT polypeptides include those from a gypsy retrotransposon, Ty4 (copia) retrotransposon, a Moloney Murine Leukemia Virus (M-MLV), a Human Immunodeficiency Virus (HIV-1), a Cauliflower Mosaic Virus (CaMV), and/or a Homo sapiens Telomerase Reverse Transcriptase. In particular embodiments, the RT polypeptide can be encoded by a nucleotide sequence that is codon optimized for a plant into which it is to be introduced and/or modified to inhibit the endogenous RNAseH activity.

LigD and Ku are enzymes in bacterial nonhomologous end-joining (NHEJ) pathways. Any known or later identified LigD and Ku polypeptides and the polynucleotides encoding them may be used with this invention to assist in DNA repair. Exemplary LigD and Ku polypeptides/polynucleotides include those from Pseudomonas aeruginosa, Mycobacterium tuberculosis, Streptomyces coelicolor, Archaeoglobus fulgidus, Bacillus subtilis, Bacillus halodurans, and/or Bordatella pertussis. In some embodiments, a LigD polypeptide and a Ku polypeptide may each be encoded by a nucleotide sequence that is codon optimized for a plant into which it is to be introduced.

As used herein, a “sequence-specific nuclease” refers to a nuclease having a recognition sequence targeting them to a specific location in a nucleic acid, resulting in the introduction of strand breaks at specific sites in the nucleic acid. Both naturally occurring and programmable sequence-specific nucleases are useful with this invention. Sequence specific nucleases are well known and include, but are not limited, to Cpf1, Cas9, meganucleases, zinc finger nucleases (ZFNs) and/or transcription activator-like effector nucleases (TALENs).

A “meganuclease” refers to an endonuclease that has a large DNA recognition site to which the nuclease binds and cuts. Meganucleases are also known as “homing endonucleases.”

Any meganuclease now known or later identified can be screened for use with this invention, including but not limited to, H-Drel, I-SceI, I-SceII, I-SceIII, I-SceIV, I-SceV, I-SceVI, I-SceVII, I-CeuI, I-CeuAIIP, I-CreI, I-CrepsbIP, I-CrepsbIIP, I-CrepsbIIIP, I-CrepsbIVP, I-TliI, I-PpoI, Pi-PspI, F-SceI, F-SceII, F-SuvI, F-CphI, F-TevI, F-TevII, I-AmaI, I-AniI, I-ChuI, I-Cmoel, I-CpaI, I-CpaII, I-CsmI, I-CvuI, I-CvuAIP, I-DdiII, I-DirI, I-DmoI, I-HmuI, I-HmuII, I-HsNIP, I-LlaI, I-MsoI, I-NaaI, I-NanI, I-NclIP, I-NgrIP, I-NitI, I-NjaI, I-Nsp236IP, I-PakI, I-PboIP, I-PcuIP, I-PcuAI, I-PcuVI, I-PgrIP, I-PobIP, I-PorI, I-PorIIP, I-PbpIP, I-SpBetalP, I-ScaI, I-SexIP, I-SneIP, I-SpomI, I-SpomCP, I-SpomIP, I-SpomIIP, I-SquIP, I-Ssp68031, I-SthPhiJP, I-SthPhiST3P, I-SthPhiSTe3bP, I-TdeIP, I-TevI, I-TevII, I-TevIII, I-UarAP, I-UarHGPAIP, I-UarHGPA13P, I-VinIP, I-ZbiIP, PI-Mgal, PI-MtuI, PI-MtuHIP PI-MtuHIIP, PI-PfuI, PI-PfuII, PI-PkoI, PI-PkoII, PI-Rma43812IP, PI-SpBetaIP, PI-Scel, PI-TfuI, PI-TfuII, PI-Thyl, PI-TliI, and/or PI-TliII.

In some embodiments, an endonuclease may bind to a native or endogenous recognition sequence. In other embodiments, the endonuclease may be modified such that it binds a non-native or exogenous recognition sequence and does not bind a native or endogenous recognition sequence. Meganucleases and known and described in, for example, U.S. Pat. No. 8,685,737; U.S. Pat. No. 8,765,448; and U.S. Pat. No.8,921,332, each of which are incorporated by reference in their entireties for the teachings relevant to meganucleases. A “transcription activator-like effector nuclease” (TALEN) is a nuclease that targets specific nucleic acid sequences for cleavage. A TALEN is produced by fusing a transcription activator-like effector DNA-binding domain to a DNA cleavage (nuclease) domain from, for example, a type II restriction endonuclease, e.g., a nonspecific cleavage domain from a type II restriction endonuclease such as Fokl. The TAL-effector DNA binding domain interacts with DNA in a sequence-specific manner through one or more tandem repeat domains and may be engineered to bind to a desired target sequence. Other useful endonucleases, in addition to FokI, may include, for example, HhaI, HindIII, Nod, BbvCI, EcoRI, BglI, and/or AlwI. Thus, in some embodiments, the TALEN comprises a TAL effector domain comprising a plurality of TAL effector repeat sequences that, in combination, bind to a specific nucleotide sequence in the target DNA sequence, such that the TALEN cleaves the target DNA within or adjacent to the specific or target nucleotide sequence. TALENs and their use are known in the art and those useful with this invention can include any that are now known or any later identified, see, for example, U.S. Pat. No. 8,685,737 and U.S. Pat. No. 9,393,257, each of which are incorporated by reference in their entireties for the teachings relevant to TALENs. Zinc-finger nucleases (ZFNs) are produced by fusing together a zinc finger DNA-binding domain (e.g., Cys₂-His₂ zinc-finger domain) and a DNA-cleavage domain (e.g., Fok1 restriction enzyme). Zinc-finger domains have been developed that recognize nearly all of the 64 possible nucleotide triplets, which allows for modular assembly of ZFNs for sequence specific targeting and editing. See, for example, U.S. Pat. No. 8,685,737 and U.S. Pat. No. 8,106,255, each of which is incorporated by reference in its entirety for the teachings relevant to ZFNs.

Also useful with this invention are chimeric endonucleases that can be produced by linking DNA binding sequence(s) and DNA cleavage domains DNA binding sequences include, for example, zinc finger binding domains and/or meganuclease recognition sites. DNA cleavage domains include restriction endonuclease cleavage domains. U.S. Pat. No. 8,921,332, which is incorporated by reference in its entireties for the teachings relevant to chimeric endonucleases.

“Cas9 polypeptide” or “Cas9 nuclease” refers to a large group of endonucleases that catalyze the double stranded DNA cleavage in the CRISPR Cas system. These polypeptides are well known in the art and many of their structures (sequences) are characterized (See, e.g., WO2013/176772; WO/2013/188638). The domains for catalyzing the cleavage of the double stranded DNA are the RuvC domain and the HNH domain. The RuvC domain is responsible for nicking the (+) strand and the HNH domain is responsible for nicking the (−) strand (See, e.g., Gasiunas et al. PNAS 109(39):E2579-E2586 (Sep. 4, 2012)). A Cas9 nuclease comprising a mutation in the RuvC endonuclease domain and a mutation in the HNH endonuclease domain, which results in the disruption of both RuvC and HNH nuclease activity is called a deactivated Cas9 (dCas9).

Any Cas9 nuclease known or later identified to catalyze DNA cleavage in a CRISPR-Cas system may be used with this invention. In some embodiments, a Cas9 polypeptide useful with this invention comprises at least 70% identity (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like) to an amino acid sequence encoding a Cas9 nuclease. As known in the art, such Cas9 nucleases comprise a HNH motif and a RuvC motif (See, e.g., W02013/176772; WO/2013/188638). In representative embodiments, a functional fragment of a Cas9 nuclease can be used with this invention. A Cas9 functional fragment retains one or more of the activities of a native Cas9 nuclease including, but not limited to, HNH nuclease activity, RuvC nuclease activity, DNA, RNA and/or PAM recognition and binding activities. A functional fragment of a Cas9 polypeptide may be encoded by a fragment of a Cas9 polynucleotide. A Cas9 polypeptide useful with this invention includes deactivated Cas9 (dCas9) polypeptides and fragments thereof (thereby eliminating nuclease activity but retaining one or more of DNA, RNA and/or PAM recognition and binding activities). In some embodiments, a dCas9 can be fused to FokI (fCas9) to assist in cleavage specificity (see, e.g., Guilinger et al. Nat. Biotechnol. 32(2):577-582 (2014) and Tsai et al. Nat. Biotechnol. 32(6):569-576 (2014)). Moreover, in particular embodiments, the Cas9 polypeptide can be encoded by a nucleotide sequence that is codon optimized for a plant comprising the target DNA.

CRISPR-Cas systems and groupings of Cas9 nucleases are well known in the art and include, for example, a Streptococcus pyogenes group of Cas9 nucleases, a Staphylococcus aureus group of Cas9 nucleases, a Neisseria meningitidis group of Cas9 nucleases, a Streptococcus thermophilus CRISPR 1 (Sth CR1) group of Cas9 nucleases, a Streptococcus thermophilus CRISPR 3 (Sth CR3) group of Cas9 nucleases, a Lactobacillus buchneri CD034 (Lb) group of Cas9 nucleases, and a Lactobacillus rhamnosus GG (Lrh) group of Cas9 nucleases. Additional Cas9 nucleases include, but are not limited to, those of Lactobacillus curvatus CRL 705. Still further Cas9 nucleases useful with this invention include, but are not limited to, a Cas9 from Lactobacillus animalis KCTC 3501, and Lactobacillus farciminis WP 010018949.1.

“Cpf1 polypeptide” or “Cpf1 nuclease” refers to a family of RNA guided endonucleases that catalyze double stranded DNA breaks in the CRISPR Cas system. These polypeptides are well known in the art and many of their structures (sequences) are characterized (See, e.g., U2UMQ6.1, WP_051666128.1). The domain for catalyzing the cleavage of the double stranded DNA is the RuvC domain. Cpf1 differs from Cas9 in that it does not possess a HNH domain and has a distinct N terminal recognition structure. The RuvC domain is responsible for nicking both the (+) strand and the (−) strand (see Zetsche, Bernd, et al. Cell 163.3 (2015): 759-771.).

Any Cpf1 nuclease known or later identified to catalyze DNA cleavage in a CRISPR-Cas system may be used with this invention. In some embodiments, a Cpf1 polypeptide useful with this invention comprises at least 70% identity (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like) to an amino acid sequence encoding a Cpf1 nuclease. As known in the art, such Cpf1 nucleases comprise a RuvC motif (See, e.g., U2UMQ6.1, WP_051666128.1). In representative embodiments, a functional fragment of a Cpf1 nuclease can be used with this invention. A Cpf1 functional fragment retains one or more of the activities of a native Cpf1 nuclease including, but not limited to, RuvC nuclease activity, CRISPR array processing, DNA, RNA and/or PAM recognition and binding activities. A functional fragment of a Cpf1 polypeptide may be encoded by a fragment of a Cpf1 polynucleotide. In particular embodiments, the Cpf1 polypeptide can be encoded by a nucleotide sequence that is codon optimized for a plant comprising the target DNA.

CRISPR-Cas systems and groupings of Cpf1 nucleases are well known in the art and include, for example, a Acidaminococcus sp. Group of Cpf1 nucleases, a Lachnospiraceae bacterium group of Cpf1 nucleases.

A “repeat sequence” as used herein refers, for example, to any repeat sequence of a wild-type CRISPR locus or a repeat sequence of a synthetic CRISPR array that are separated by “spacer sequences.” A repeat sequence useful with this invention can be any known or later identified repeat sequence of a CRISPR locus or it can be a synthetic repeat designed to function in a CRISPR Type II system. Accordingly, in some embodiments, a spacer-repeat sequence, a repeat-spacer-repeat, or CRISPR array can comprise a repeat that is substantially identical (e.g. at least about 70% identical (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a repeat from a wild-type Type II CRISPR array or a Type V CRISPR array. In some embodiments, a repeat sequence may be 100% identical to a repeat from a wild-type CRISPR array. In additional embodiments, a repeat sequence useful with this invention may comprise a nucleotide sequence comprising a partial repeat that is a fragment or portion of consecutive nucleotides of a repeat sequence of a CRISPR locus or synthetic CRISPR array.

A “CRISPR spacer-repeat nucleic acid” as used herein comprises a spacer sequence as described herein that is linked to the 5′ end of a repeat sequence as described herein. Optionally, a CRISPR spacer-repeat nucleic acid can further comprise a repeat sequence linked to the 5′ end of the spacer-repeat nucleic acid (i.e., linked to the 5′ end of the spacer of the spacer-repeat nucleic acid).

A “CRISPR array” as used herein means a nucleic acid molecule that comprises two or more repeat sequences, or a portion of each of said repeat sequences, and at least one spacer sequence, wherein one of the two or more repeat sequences, or said portion thereof, is linked to the 5′ end of the spacer sequence and the other of the two repeat sequences, or portion thereof, is linked to the 3′ end of the spacer sequence. In a recombinant CRISPR array, the combination of repeat sequences and spacer sequences is synthetic, made by man and not found in nature. In some embodiments, a “CRISPR array” refers to a nucleic acid construct that comprises from 5′ to 3′ at least one spacer-repeat sequence (e.g., 1, 2, 3, 4, 5, 6, 7, 8, 9, 10, 11, 12, 13, 14, 15, 16, 17, 18, 19, 20, 21, 22, 23, 24, 25 or more spacer-repeat sequences, and any range or value therein), wherein the 5′ end of the 5′ most spacer-repeat of the array is linked to a repeat sequence, thereby all spacers in said array are flanked on both the 5′ end and the 3′ end by a repeat sequence.

A CRISPR array of the invention can be of any length and comprise any number of spacer sequences alternating with repeat sequences, as described above. In some embodiments, a CRISPR array can comprise, consist essentially of, or consist of 1 to about 100 spacer sequences, each linked on its 5′ end and its 3′ end to a repeat sequence (e.g., repeat-spacer-repeat-spacer-repeat-spacer-repeat-spacer-repeat, and so on, so that each CRISPR array begins and ends with a repeat). Thus, in some embodiments, a recombinant CRISPR array of the invention can comprise, consist essentially of, or consist of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more, spacer sequences each linked on its 5′ end and its 3′ end to a repeat sequence.

In some embodiments, the repeat sequence of a first CRISPR spacer-repeat nucleic acid can be operably linked directly to the 5′ end of a spacer of a second CRISPR spacer-repeat nucleic acid (i.e., no linking nucleotides) or via at least one linking nucleotide (e.g., about 1 to about 100 or more nucleotides).

In some embodiments, a CRISPR spacer-repeat nucleic acid or CRISPR array can be linked with a tracr nucleic acid (tracrDNA, tracrRNA) to form a single guide nucleic acid (sgDNA, sgRNA).

A “protospacer sequence” refers to the target double stranded DNA and specifically to the portion of the target DNA (e.g., or target region in the genome) that is fully or substantially complementary (and hybridizes) to the spacer sequence of a CRISPR spacer-repeat sequence, a CRISPR spacer-repeat-repeat sequence, and/or a CRISPR array. The protospacer sequence is next to a protospacer-adjacent motif (PAM) (PAM is 5′ to the protospacer for Cpf1 and PAM is 5′ to the protospacer for Cas9) that is recognized by the Cas9 protein or the Cpf1 protein. Protospacer-adjacent motifs are either known in the art and/or can be determined through established methods.

A “spacer sequence” as used herein is a nucleotide sequence that is complementary to a target DNA (i.e., target region in the plastid genome or the “protospacer sequence,” which is flanked by a protospacer adjacent motif (PAM) sequence, which is immediately 3′ of the protospacer or target DNA sequence for Cas9 or). The spacer sequence can be fully complementary or substantially complementary (e.g., at least about 70% complementary (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a target DNA. In representative embodiments, the spacer sequence has 100% complementarity to the target DNA. In some embodiments, the complementarity of the 3′ region of the spacer sequence to the target DNA is 100% but is less than 100% in the 5′ region of the spacer and therefore the overall complementarity of the spacer sequence to the target DNA is less than 100%. Thus, for example, the first 7, 8, 9, 10, 11, 12, 13, 14, 15, 16, and the like, nucleotides in the 3′ region of a 20 nucleotide spacer sequence (seed sequence) can be 100% complementary to the target DNA, while the remaining nucleotides in the 5′ region of the spacer sequence are substantially complementary (e.g., at least about 70% complementary) to the target DNA. In some embodiments, the last 7 to 12 nucleotides (5′ to 3′) of the spacer sequence can be 100% complementary to the target DNA, while the remaining nucleotides in the 5′ region of the spacer sequence are substantially complementary (e.g., at least about 70% complementary) to the target DNA. In other embodiments, the last 7 to 10 nucleotides of the spacer sequence can be 100% complementary to the target DNA, while the remaining nucleotides in the 5′ region of the spacer sequence are substantially complementary (e.g., at least about 70% complementary) to the target DNA. In representative embodiments, the last 7 nucleotides (within the seed) of the spacer sequence can be 100% complementary to the target DNA, while the remaining nucleotides in the 5′ region of the spacer sequence are substantially complementary (e.g., at least about 70% complementary) to the target DNA.

In representative embodiments, a spacer sequence of a CRISPR spacer-repeat nucleic acid of the invention comprises at least about 16 consecutive nucleotides of a target nucleic acid, wherein at the 3′ end of said spacer at least about 10 consecutive nucleotides of said at least about 16 consecutive nucleotides comprise at least about 90% complementarity to the target nucleic acid, wherein the target nucleic acid is adjacent to a protospacer adjacent motif (PAM) sequence in the genome of an organism of interest.

As used herein, a “target DNA,” “target region” or a “target region in the genome” refers to a region of an organism's genome that is fully complementary or substantially complementary (e.g., at least 70% complementary (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a spacer sequence in a spacer-repeat sequence or repeat-spacer-repeat sequence. In some embodiments, a target region may be about 10 to about 30 consecutive nucleotides, about 10 to about 40 consecutive nucleotides, about 10 to about 50 nucleotides, about 10 to about 60 nucleotides, about 10 to about 70 nucleotides, about 10 to about 80 nucleotides, about 10 to about 90 nucleotides, or about 10 to about 100 nucleotides, or more in length located immediately adjacent to a PAM sequence (PAM sequence located immediately 3′ or 5′ of the target region (protospacer) depending on the nuclease (e.g., Cpf1 or Cas9)) in the plastid genome of the organism.

A “hairpin sequence” as used herein, is a nucleotide sequence comprising hairpins (e.g., that forms one or more hairpin structures). A hairpin (e.g., stem-loop, fold-back) refers to a nucleic acid molecule having a secondary structure that includes a region of complementary nucleotides that form a double strand with an unstructured loop. Such structures are well known in the art. As known in the art, the double stranded region can comprise some mismatches in base pairing or can be perfectly complementary. In some embodiments of the present disclosure, a hairpin sequence of a nucleic acid construct can be located at the 3′end of a tracr nucleic acid.

A “trans-activating CRISPR (tracr) nucleic acid” or “tracr nucleic acid” as used herein refers to any tracr RNA (or its encoding DNA). A tracr nucleic acid comprises from 5′ to 3′ a bulge, a lower stem, a nexus hairpin and terminal hairpins, and optionally, at the 5′ end, an upper stem (See, Briner et al. (2014) Molecular Cell. 56(2):333-339).

A trans-activating CRISPR (tracr) nucleic acid functions in Type II CRISPR systems by hybridizing to the repeat portion of mature or immature crRNAs and recruiting Cas9 protein to the target site. The tracr nucleic acid may facilitate the catalytic activity of Cas9 by inducting structural rearrangement. Sequences for tracrRNAs are specific to the CRISPR-Cas system and can be variable. Any tracr nucleic acid, known or later identified, can be used with this invention. Thus, in some embodiments, a tracr nucleic acid useful with the invention can be any Type II CRISPR tracr nucleic acid and the Cas9 nuclease can be a Cas9 nuclease that corresponds to the tracr nucleic acid that is chosen. A “minimal tracr nucleic acid” comprises from 5′ to 3′ a bulge, a lower stem, a nexus hairpin, and terminal hairpins. In some embodiments, a tracr nucleic acid or a minimal tracr nucleic acid may be linked to a spacer-repeat sequence, repeat-spacer-repeat sequence, or to a CRISPR array to form a single guide nucleic acid (sgRNA, sgDNA).

As used herein “sequence identity” refers to the extent to which two optimally aligned polynucleotide or peptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the phrase “substantially identical,” or “substantial identity” in the context of at least two nucleic acid molecules, nucleotide sequences or protein sequences, refers to two or more sequences or subsequences that have at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and/or 100% nucleotide or amino acid residue identity, when compared and aligned for maximum correspondence, as measured using one of the following sequence comparison algorithms or by visual inspection. In some embodiments of the invention, the substantial identity exists over a region of the sequences that is at least about 50 nucleotides/residues to about 150 nucleotides/residues in length. Thus, in some embodiments of the invention, the substantial identity exists over a region of the sequences that is at least about 3 to about 15 (e.g., 3, 4, 5, 6, 7, 8, 9, 10, 11, 12, 13, 14, 15 nucleotides/residues in length and the like, or any value or any range therein), at least about 5 to about 30 , at least about 10 to about 30, at least about 16 to about 30, at least about 18 to at least about 25, at least about 18, at least about 22, at least about 25, at least about 30, at least about 40, at least about 50, about 60, about 70, about 80, about 90, about 100, about 110, about 120, about 130, about 140, about 150, or more nucleotides/residues in length, and any range therein.

In representative embodiments, the sequences can be substantially identical over at least about 22 nucleotides. In some embodiments, sequences of the invention can be about 70% to about 100% identical over at least about 16 nucleotides to about 25 nucleotides. In some embodiments, sequences of the invention can be about 75% to about 100% identical over at least about 16 nucleotides to about 25 nucleotides. In further embodiments, sequences of the invention can be about 80% to about 100% identical over at least about 16 nucleotides to about 25 nucleotides. In further embodiments, sequences of the invention can be about 80% to about 100% identical over at least about 7 nucleotides to about 25 nucleotides. In some embodiments, sequences of the invention can be about 70% identical over at least about 18 nucleotides. In some embodiments, the sequences can be about 85% identical over about 22 nucleotides. In some embodiments, the sequences can be 100% identical over about 16 nucleotides. In some embodiments, the sequences are substantially identical over the entire length of a coding region. Furthermore, in some embodiments, substantially identical nucleotide or polypeptide sequences perform substantially the same function (e.g., the function or activity of a sequence-specific nuclease (e.g., meganuclease, Cpf1 nuclease (e.g., nickase, DNA, RNA and/or PAM recognition and binding activities), Cas9 nuclease (e.g., nickase, DNA, RNA and/or PAM recognition and binding activites), ZFN, TALEN), a reverse transcriptase, a Ku polypeptide, a LigD polypeptide, a tracr nucleic acid, and/or a repeat sequence.

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, Calif.). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences may be to a full-length polynucleotide sequence or a portion thereof, or to a longer polynucleotide sequence. For purposes of this invention “percent identity” may also be determined using BLASTX version 2.0 for translated nucleotide sequences and BLASTN version 2.0 for polynucleotide sequences.

Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89: 10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleotide sequence to the reference nucleotide sequence is less than about 0.1 to less than about 0.001. Thus, in some embodiments of the invention, the smallest sum probability in a comparison of the test nucleotide sequence to the reference nucleotide sequence is less than about 0.001.

Two nucleotide sequences can also be considered to be substantially complementary when the two sequences hybridize to each other under stringent conditions. In some representative embodiments, two nucleotide sequences considered to be substantially complementary hybridize to each other under highly stringent conditions.

“Stringent hybridization conditions” and “stringent hybridization wash conditions” in the context of nucleic acid hybridization experiments such as Southern and Northern hybridizations are sequence dependent, and are different under different environmental parameters. An extensive guide to the hybridization of nucleic acids is found in Tijssen Laboratory Techniques in Biochemistry and Molecular Biology-Hybridization with Nucleic Acid Probes part I chapter 2 “Overview of principles of hybridization and the strategy of nucleic acid probe assays” Elsevier, New York (1993). Generally, highly stringent hybridization and wash conditions are selected to be about 5° C. lower than the thermal melting point (Tₘ) for the specific sequence at a defined ionic strength and pH.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleotide sequences which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2× SSC wash at 65° C. for 15 minutes (see, Sambrook, infra, for a description of SSC buffer). Often, a high stringency wash is preceded by a low stringency wash to remove background probe signal. An example of a medium stringency wash for a duplex of, e.g., more than 100 nucleotides, is lx SSC at 45° C. for 15 minutes. An example of a low stringency wash for a duplex of, e.g., more than 100 nucleotides, is 4-6× SSC at 40° C. for 15 minutes. For short probes (e.g., about 10 to 50 nucleotides), stringent conditions typically involve salt concentrations of less than about 1.0 M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2× (or higher) than that observed for an unrelated probe in the particular hybridization assay indicates detection of a specific hybridization. Nucleotide sequences that do not hybridize to each other under stringent conditions are still substantially identical if the proteins that they encode are substantially identical. This can occur, for example, when a copy of a nucleotide sequence is created using the maximum codon degeneracy permitted by the genetic code. The following are examples of sets of hybridization/wash conditions that may be used to clone homologous nucleotide sequences that are substantially identical to reference nucleotide sequences of the invention. In one embodiment, a reference nucleotide sequence hybridizes to the “test” nucleotide sequence in 7% sodium dodecyl sulfate (SDS), 0.5 M NaPO₄, 1 mM EDTA at 50° C. with washing in 2× SSC, 0.1% SDS at 50° C. In another embodiment, the reference nucleotide sequence hybridizes to the “test” nucleotide sequence in 7% sodium dodecyl sulfate (SDS), 0.5 M NaPO₄, 1 mM EDTA at 50° C. with washing in 1× SSC, 0.1% SDS at 50° C. or in 7% sodium dodecyl sulfate (SDS), 0.5 M NaPO₄, 1 mM EDTA at 50° C. with washing in 0.5× SSC, 0.1% SDS at 50° C. In still further embodiments, the reference nucleotide sequence hybridizes to the “test” nucleotide sequence in 7% sodium dodecyl sulfate (SDS), 0.5 M NaPO₄, 1 mM EDTA at 50° C. with washing in 0.1× SSC, 0.1% SDS at 50° C., or in 7% sodium dodecyl sulfate (SDS), 0.5 M NaPO₄, 1 mM EDTA at 50° C. with washing in 0.1× SSC, 0.1% SDS at 65° C.

As is well known in the art, nucleotide sequences can be codon optimized for expression in any species of interest. Codon optimization is well known in the art and involves modification of a nucleotide sequence for codon usage bias using species specific codon usage tables. The codon usage tables are generated based on a sequence analysis of the most highly expressed genes for the species of interest. When the nucleotide sequences are to be expressed in the nucleus, the codon usage tables are generated based on a sequence analysis of highly expressed nuclear genes for the species of interest. The modifications of the nucleotide sequences are determined by comparing the species specific codon usage table with the codons present in the native polynucleotide sequences. As is understood in the art, codon optimization of a nucleotide sequence results in a nucleotide sequence having less than 100% identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like) to the native nucleotide sequence but which still encodes a polypeptide having the same function as that encoded by the original, native nucleotide sequence. Thus, in some embodiments of the invention, a nucleotide sequence, nucleic acid and/or nucleic acid construct of this invention can be codon optimized for expression in the particular species of interest. In some embodiments, a polynucleotide encoding a sequence-specific nuclease (e.g., Cpf1 nuclease, Cas9 nuclease, ZFN. TALEN, meganuclease) can be codon optimized for expression in an organism of interest. Thus, for example, Cas9 or a Cpf1 polypeptide may be codon optimized for expression in Zea mays or Chlamydomonas reinhardtii. As an example, a codon optimized Cas9 polypeptide has been shown to be functional in Arabidopsis. (See, e.g., Jiang et al. Nucleic Acids Research, 41(20), el 88. (2013) and Xing et al. BMC Plant Biology 14:327 (2014)).

In some embodiments, the nucleic acids, nucleotide sequences and/or polypeptides of the invention are “isolated.” An “isolated” nucleic acid, an “isolated” nucleotide sequence or an “isolated” polypeptide is a nucleic acid, nucleotide sequence or polypeptide that, by the human hand, exists apart from its native environment and is therefore not a product of nature.

An isolated nucleic acid, nucleotide sequence or polypeptide may exist in a purified form that is at least partially separated from at least some of the other components of the naturally occurring organism or virus, for example, the cell or viral structural components or other polypeptides or nucleic acids commonly found associated with the polynucleotide. In representative embodiments, the isolated nucleic acid, the isolated nucleotide sequence and/or the isolated polypeptide is at least about 1%, 5%, 10%, 20%, 30%, 40%, 50%, 60%, 70%, 80%, 90%, 95%, or more pure.

In other embodiments, an isolated nucleic acid, nucleotide sequence or polypeptide may exist in a non-native environment such as, for example, a recombinant host cell. Thus, for example, with respect to nucleotide sequences, the term “isolated” means that it is separated from the chromosome and/or cell in which it naturally occurs. A polynucleotide is also isolated if it is separated from the chromosome and/or cell in which it naturally occurs in and is then inserted into a genetic context, a chromosome and/or a cell in which it does not naturally occur (e.g., a different host cell, different regulatory sequences, and/or different position in the genome than as found in nature). Accordingly, the recombinant nucleic acids, nucleotide sequences and their encoded polypeptides are “isolated” in that, by the human hand, they exist apart from their native environment and therefore are not products of nature. However, in some embodiments, they can be introduced into and exist in a recombinant host cell.

In some embodiments, the nucleotide sequences, polynucleotides, nucleic acids, and nucleic acid constructs of the invention can be “synthetic.” A “synthetic” nucleic acid, a “synthetic” nucleotide sequence or a “synthetic” polynucleotide is a nucleic acid, nucleotide sequence or polynucleotide that is not found in nature but is created by the human hand and is therefore not a product of nature.

In any of the embodiments described herein, the nucleotide sequences, polynucleotides, nucleic acids, and nucleic acid constructs of the invention can be operably associated with a variety of promoters, terminators, and/or other regulatory elements for expression in plant cell. Any promoter, terminator or other regulatory element functional in a plant cell may be used with the nucleic acids of this invention. A promoter useful with this invention may be a constitutive, inducible, temporally regulated, developmentally regulated, tissue specific or tissue preferred promoter. Thus, in representative embodiments, a promoter may be operably linked to a polynucleotide and/or nucleic acid of the invention (e.g., a polynucleotide encoding a sequence-specific nuclease (e.g., Cpf1 nuclease, Cas9 nuclease, meganuclease, ZFN, TALEN), a polynucleotide encoding a reverse transcriptase (RT) polypeptide, a polynucleotide encoding LigD, a polynucleotide encoding Ku, a guide nucleic acid (a CRISPR spacer-repeat sequence, tracr nucleic acid, CRISPR array, a tracr nucleic acid fused to a CRISPR array (sgDNA,sgRNA)), and/or a recombinant nucleic acid comprising (i) a plastid modification cassette, (ii) a first recognition site located immediately 5′ of the plastid modification cassette, (iii) a second recognition site located immediately 3′ of the plastid modification cassette, and (iv) a plastid localization sequence operably located 5′ of the first recognition site). In some embodiments, a terminator may be operably linked to a polynucleotide and/or nucleic acid of the invention.

A “promoter” is a nucleotide sequence that controls or regulates the transcription of a nucleotide sequence (i.e., a coding sequence) that is operably associated with the promoter. The coding sequence may encode a polypeptide and/or a functional RNA. Typically, a “promoter” refers to a nucleotide sequence that contains a binding site for RNA polymerase II or RNA polymerase III and directs the initiation of transcription. In general, promoters are found 5′, or upstream, relative to the start of the coding region of the corresponding coding sequence. The promoter region may comprise other elements that act as regulators of gene expression. These include a TATA box consensus sequence, and often a CAAT box consensus sequence (Breathnach and Chambon, (1981) Annu. Rev. Biochem. 50:349). In plants, the CAAT box may be substituted by the AGGA box (Messing et al., (1983) in Genetic Engineering of Plants, T. Kosuge, C. Meredith and A. Hollaender (eds.), Plenum Press, pp. 211-227). In some embodiments, when expressing a functional nucleic acid (e.g., CRISPR RNAs (e.g., sgRNAs)) to be transported to a chloroplast, a useful promoter may be a promoter recognized by RNA polymerase II (e.g., Nos promoter, CaMV 35S promoter).

Promoters useful with this invention can include, for example, constitutive, inducible, temporally regulated, developmentally regulated, chemically regulated, tissue-preferred and/or tissue-specific promoters for use in the preparation of recombinant nucleic acid molecules, i.e., “chimeric genes” or “chimeric polynucleotides.” These various types of promoters are known in the art. The choice of promoter will vary depending on the temporal and spatial requirements for expression, and also depending on the host cell to be transformed. Promoters for many different organisms are well known in the art. Based on the extensive knowledge present in the art, the appropriate promoter can be selected for the particular host organism of interest. Thus, for example, much is known about promoters upstream of highly constitutively expressed genes in model organisms and such knowledge can be readily accessed and implemented in other systems as appropriate.

In embodiments described herein, one or more of the polynucleotides and nucleic acids of the invention may be operably associated with a promoter as well as a terminator, and/or other regulatory elements for expression in plant cell. Any promoter, terminator or other regulatory element that is functional in a plant cell may be used with the nucleic acids of this invention. Non-limiting examples of promoters useful with this invention include, but are not limited to, an U6 RNA polymerase III promoter from, for example, Arabidopsis thaliana, a Nos promoter, a 35S promoter, actin promoter, ubiquitin promoter, Rubisco small subunit promoter, an inducible promoter, including but not limited to, a an AlcR/AlcA (ethanol inducible) promoter, a glucocorticoid receptor (GR) fusion, GVG, a pOp/LhGR (dexamethasone inducible) promoter, a XVE/OlexA (β-estradiol inducible) promoter, a heat shock promoter and/or a bidirectional promoter (See, e.g., Gatz, Christine. Current Opinion in Biotechnology 7(2):168-172 (1996); Borghi L. Methods Mol Bio1.655:65-75(2010); Baron et al. Nucleic acids research 23(17) (1995), 3605; Kumar et al. Plant molecular biology 87(4-5):341-353 (2015)).

By “operably linked” or “operably associated” as used herein, it is meant that the indicated elements are functionally related to each other, and are also generally physically related. Thus, the term “operably linked”, “operably located”, or “operably associated” as used herein, refers to nucleotide sequences on a single nucleic acid molecule that are functionally associated. Thus, a first nucleotide sequence that is operably linked to a second nucleotide sequence means a situation when the first nucleotide sequence is placed in a functional relationship with the second nucleotide sequence. For instance, a promoter is operably associated with a nucleotide sequence if the promoter effects the transcription or expression of said nucleotide sequence. Those skilled in the art will appreciate that the control sequences (e.g., promoter) need not be contiguous with the nucleotide sequence to which it is operably associated, as long as the control sequences function to direct the expression thereof. Thus, for example, intervening untranslated, yet transcribed, sequences can be present between a promoter and a nucleotide sequence, and the promoter can still be considered “operably linked” to the nucleotide sequence.

In some embodiments, a nucleic acid construct of the invention can be an “expression cassette” or can be comprised within an expression cassette. As used herein, “expression cassette” means a recombinant nucleic acid molecule comprising a nucleotide sequence of interest (NOI). An NOI can include, but is not limited to, a polynucleotide encoding a sequence-specific nuclease (e.g., Cpf1 nuclease, Cas9 nuclease, ZFN, TALEN, meganuclease), a polynucleotide encoding a reverse transcriptase (RT) polypeptide, a polynucleotide encoding LigD, a polynucleotide encoding a Ku, a CRISPR spacer-repeat sequence, tracr nucleic acid, CRISPR array, a tracr nucleic acid fused to a CRISPR array (to form a single guide nucleic acid), and/or a recombinant nucleic acid comprising (i) a plastid modification cassette, (ii) a first recognition site located immediately 5′ of the plastid modification cassette, (iii) a second recognition site located immediately 3′ of the plastid modification cassette, and (iv) a plastid localization sequence operably located 5′ of the first recognition site), wherein said nucleotide sequence is operably associated with at least a control sequence (e.g., a promoter). Thus, some aspects of the invention provide expression cassettes designed to express the nucleic acids constructs of the invention.

An expression cassette may be chimeric, meaning that at least one of its components is heterologous with respect to at least one of its other components. An expression cassette may also be one that is naturally occurring but has been obtained in a recombinant form useful for heterologous expression.

In addition to promoters, an expression cassette also can optionally include additionally regulatory elements functional in a plant cell including, but not limited to, a transcriptional and/or translational termination region (i.e., termination region). A variety of transcriptional terminators are available for use in expression cassettes and are responsible for the termination of transcription beyond the heterologous nucleotide sequence of interest and correct mRNA polyadenylation. The termination region may be native to the transcriptional initiation region, may be native to the operably linked nucleotide sequence of interest, may be native to the host cell, or may be derived from another source (i.e., foreign or heterologous to the promoter, to the nucleotide sequence of interest, to the host, or any combination thereof). Non-limiting examples of terminators functional in a plant and useful with this invention include, but are not limited to, an actin terminator; a Rubisco small subunit terminator, a Rubisco large subunit terminator, a nopaline synthase (nos) terminator, and/or a ubiquitin terminator.

An expression cassette also can include a nucleotide sequence for a selectable marker, which can be used to select a transformed host cell. As used herein, “selectable marker” means a nucleotide sequence that when expressed imparts a distinct phenotype to the host cell expressing the marker and thus allows such transformed cells to be distinguished from those that do not have the marker. Such a nucleotide sequence may encode either a selectable or screenable marker, depending on whether the marker confers a trait that can be selected for by chemical means, such as by using a selective agent (e.g., an antibiotic and the like), or on whether the marker is simply a trait that one can identify through observation or testing, such as by screening (e.g., fluorescence). Many examples of suitable selectable markers are known in the art and can be used in the expression cassettes described herein.

In addition to expression cassettes, the nucleic acids described herein can be used in connection with vectors. The term “vector” refers to a composition for transferring, delivering or introducing one or more nucleic acids into a cell. A vector comprises a nucleic acid molecule comprising the nucleotide sequence(s) to be transferred, delivered or introduced. Vectors for use in transformation of host organisms are well known in the art. Non-limiting examples of general classes of vectors include but are not limited to a viral vector, a plasmid vector, a phage vector, a phagemid vector, a cosmid vector, a fosmid vector, a bacteriophage, an artificial chromosome, or an Agrobacterium binary vector in double or single stranded linear or circular form which may or may not be self transmissible or mobilizable. A vector as defined herein can transform a eukaryotic host either by integration into the cellular genome or exist extrachromosomally (e.g. autonomous replicating plasmid with an origin of replication). Additionally included are shuttle vectors by which is meant a DNA vehicle capable, naturally or by design, of replication in two different host organisms. In some representative embodiments, the nucleic acid in the vector is under the control of, and operably linked to, an appropriate promoter or other regulatory elements for transcription in a host cell. The vector may be a bi-functional expression vector which functions in multiple hosts. In the case of genomic DNA, this may contain its own promoter or other regulatory elements and in the case of cDNA this may be under the control of an appropriate promoter or other regulatory elements for expression in the host cell. Accordingly, the nucleic acid molecules of this invention and/or expression cassettes can be comprised in vectors as described herein and as known in the art.

“Introducing,” “introduce,” “introduced” (and grammatical variations thereof) in the context of a polynucleotide of interest (e.g., any nucleic acid or polynucleotide of the invention) means presenting the polynucleotide of interest to the host organism or cell of said organism (e.g., host cell) in such a manner that the polynucleotide gains access to the interior of a cell. Where more than one polynucleotide is to be introduced these polynucleotides can be assembled as part of a single polynucleotide or nucleic acid construct, or as separate polynucleotides or nucleic acid constructs, and can be located on the same or different expression constructs or transformation vectors. Accordingly, these polynucleotides can be introduced into cells in a single transformation event, in separate transformation/transfection events, or, for example, they can be incorporated into an organism by conventional breeding protocols. Thus, in some aspects, one or more nucleic acid constructs of the invention (e.g., a polynucleotide encoding a sequence-specific nuclease (e.g., Cpf1 nuclease, Cas9 nuclease, meganuclease, ZFN, TALEN), a polynucleotide encoding a reverse transcriptase (RT) polypeptide, a polynucleotide encoding LigD, a polynucleotide encoding Ku, a guide nucleic acid (a CRISPR spacer-repeat sequence, tracr nucleic acid, CRISPR array, a tracr nucleic acid fused to a CRISPR array (sgDNA,sgRNA)), and/or a recombinant nucleic acid comprising (i) a plastid modification cassette, (ii) a first recognition site located immediately 5′ of the plastid modification cassette, (iii) a second recognition site located immediately 3′ of the plastid modification cassette, and (iv) a plastid localization sequence operably located 5′ of the first recognition site) can be introduced singly or in combination in a single expression cassette and/or vector into a host organism or a cell of said host organism.

The term “transformation” or “transfection” as used herein refers to the introduction of a heterologous nucleic acid into a cell. Transformation of a cell may be stable or transient or may be in part stably transformed and in part transiently transformed. Thus, in some embodiments, the modifications to the plastid genome can be stable and in some embodiments, the modifications to the nuclear genome can be transient. In some embodiments, after stable transformation or modification of the plastid genome, the nucleic acid constructs introduced to the nuclear genome can be removed by, for example, crossing with non-modified plants or segregation of non-homozygous plants.

“Transient transformation” in the context of a polynucleotide means that a polynucleotide is introduced into the cell and does not integrate into the nuclear or plastid genome of the cell.

By “stably introducing” or “stably introduced,” in the context of a polynucleotide, means that the introduced polynucleotide is stably incorporated into the genome of the cell, and thus the cell is stably transformed with the polynucleotide.

“Stable transformation” or “stably transformed” as used herein means that a nucleic acid construct is introduced into a cell and integrates into the genome of the cell. As such, the integrated nucleic acid construct is capable of being inherited by the progeny thereof, more particularly, by the progeny of multiple successive generations. “Genome” as used herein can include the nuclear, plastid, and/or mitochondrial genome, and therefore may include integration of a nucleic acid construct into the nuclear, plastid and/or mitochondrial genome. Stable transformation as used herein may also refer to a transgene that is maintained extrachromasomally, for example, as a minichromosome or a plasmid.

Transient transformation may be detected by, for example, an enzyme-linked immunosorbent assay (ELISA) or Western blot, which can detect the presence of a peptide or polypeptide encoded by one or more transgene introduced into a plant or plant cell. Stable transformation of a cell can be detected by, for example, a Southern blot hybridization assay of genomic DNA of the cell with nucleic acid sequences which specifically hybridize with a nucleotide sequence of a transgene introduced into an organism (e.g., a bacterium, an archaea, a yeast, an algae, and the like). Stable transformation of a cell can be detected by, for example, a Southern blot hybridization assay of DNA of the cell with nucleic acid sequences which specifically hybridize with a nucleotide sequence of a transgene introduced into a plant or other organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods. Transformation can also be detected by direct sequencing and/or hybridization protocols well known in the art.

A heterologous nucleotide sequence or nucleic acid construct of the invention can be introduced into a cell by any method known to those of skill in the art. In some embodiments of the invention, transformation of a cell comprises nuclear transformation. In some embodiments of the invention, transformation of a cell comprises plasmid transformation. In some embodiments, the nuclear transformation can be transient, while the plastid transformation or modification is stably integrated into the plastid genome. In still further embodiments, the heterologous nucleotide sequence(s) or nucleic acid construct(s) of the invention can be introduced into a cell via conventional breeding techniques.

Procedures for transforming both eukaryotic and prokaryotic organisms are well known and routine in the art and are described throughout the literature (See, for example, Jiang et al. 2013. Nat. Biotechnol. 31:233-239; Ran et al. Nature Protocols 8:2281-2308 (2013)). A nucleotide sequence therefore can be introduced into a host organism or its cell in any number of ways that are well known in the art. The methods of the invention do not depend on a particular method for introducing one or more nucleotide sequences into the organism, only that they gain access to the interior of at least one cell of the organism. Where more than one nucleotide sequence is to be introduced, they can be assembled as part of a single nucleic acid construct, or as separate nucleic acid constructs, and can be located on the same or different nucleic acid constructs. Accordingly, the nucleotide sequences can be introduced into the cell of interest in a single transformation event, or in separate transformation events, or, alternatively, where relevant, a nucleotide sequence can be incorporated into an organism as part of a breeding protocol.

The present invention provides novel nucleic acid constructs and methods for modifying a plastid genome. In some embodiments, a plant or plant cell can be transformed with nucleic acid constructs of this invention that are imported into plastids directly (utilizing plastid localization sequences) or when expressed the polypeptide products of said nucleic acid constructs are imported into plastids (utilizing plastid transit peptides), whereby the plastid genome is modified.

In some embodiments, homologous repair and recombination may be used to modify a plastid genome. Thus, for example, to introduce a polypeptide of interest (POI) into a plastid by homologous recombination, a reverse transcriptase and a plastid modification cassette comprising an intervening sequence comprising a POI as described herein can be introduced into a plant cell. In some embodiments, to improve the efficiency of recombination, the plant cell can be further transformed with a sequence-specific nuclease (e.g., Cpf1 nuclease, meganuclease, ZFN, TALEN, and/or a Cas9 and guide nucleic acid). The double stranded DNA generated by the reverse transcriptase will serve as a template during the homologous repair and recombination mechanism. The sequence-specific nuclease (e.g., Cpf1 nuclease, CRISPR-Cas9 system, meganuclease, ZFN, TALEN) will try to create double strand break in the plastid genome and the polynucleotide of interest in the plastid modification cassette will have the flanking sequences of the target region in the plastid DNA for homologous recombination during the repair process. Similarly, a deletion can be generated using a plastid modification cassette that does not comprise an intervening sequence. These and other methods of modifying a plastid genome and producing plants and plant cells comprising modified plastid genomes are described herein.

Thus, in some embodiments, a method of modifying a plastid genome of a plant cell is provided, comprising, consisting essentially of, or consisting of: introducing into a plant cell introducing into a plant cell (a) a polynucleotide encoding a reverse transcriptase (RT) polypeptide fused to a plastid transit peptide; (b) a recombinant nucleic acid comprising: (i) a plastid modification cassette, (ii) a first recognition site (i.e., long terminal repeat (LTR)) located immediately 5′ of the plastid modification cassette, (iii) a second recognition site located immediately 3′ of the plastid modification cassette, and (iv) a plastid localization sequence operably located 5′ of the first recognition site; and (c) a polynucleotide encoding a sequence-specific nuclease fused to a plastid transit peptide, thereby modifying the plastid genome of said plant cell.

In some embodiments, a method of modifying a plastid genome of a plant cell is provided, comprising, consisting essentially of, or consisting of: introducing into a plant cell introducing into a plant cell (a) a polynucleotide encoding a reverse transcriptase (RT) polypeptide fused to a plastid transit peptide; (b) a recombinant nucleic acid comprising: (i) a plastid modification cassette, (ii) a first recognition site (i.e., long terminal repeat (LTR)) located immediately 5′ of the plastid modification cassette, (iii) a second recognition site located immediately 3′ of the plastid modification cassette, and (iv) a plastid localization sequence operably located 5′ of the first recognition site; and (c) polynucleotide encoding a Cas9 nuclease fused to a plastid transit peptide; and a guide nucleic acid linked to a plastid localization sequence, thereby modifying the plastid genome of said plant cell.

In some embodiments, a method of modifying a plastid genome of a plant cell is provided, comprising, consisting essentially of, or consisting of: introducing into a plant cell (a) a polynucleotide encoding a reverse transcriptase (RT) polypeptide fused to a plastid transit peptide; (b) a recombinant nucleic acid comprising: (i) a plastid modification cassette, (ii) a first recognition site (i.e., long terminal repeat (LTR)) located immediately 5′ of the plastid modification cassette, (iii) a second recognition site located immediately 3′ of the plastid modification cassette, and (iv) a plastid localization sequence operably located 5′ of the first recognition site; and (c) polynucleotide encoding a Cpf1 nuclease fused to a plastid transit peptide; and a guide (crRNA, crDNA) nucleic acid linked to a plastid localization sequence, thereby modifying the plastid genome of said plant cell.

In some embodiments, a method of modifying a plastid genome of a plant cell is provided, comprising, consisting essentially of, or consisting of: introducing into a plant cell introducing into a plant cell (a) a polynucleotide encoding a reverse transcriptase (RT) polypeptide fused to a plastid transit peptide; (b) a recombinant nucleic acid comprising: (i) a plastid modification cassette, (ii) a first recognition site (i.e., long terminal repeat (LTR)) located immediately 5′ of the plastid modification cassette, (iii) a second recognition site located immediately 3′ of the plastid modification cassette, and (iv) a plastid localization sequence operably located 5′ of the first recognition site; and (c) a polynucleotide encoding a transcription activator-like (TAL) effector nuclease (TALEN) fused to a plastid transit peptide, wherein the TALEN comprises a TAL effector DNA-binding domain fused to a DNA cleavage domain, thereby modifying the plastid genome of said plant cell.

In still further embodiments, a method of modifying a plastid genome of a plant cell is provided, comprising, consisting essentially of, or consisting of: introducing into a plant cell introducing into a plant cell (a) a polynucleotide encoding a reverse transcriptase (RT) polypeptide fused to a plastid transit peptide; (b) a recombinant nucleic acid comprising: (i) a plastid modification cassette, (ii) a first recognition site (i.e., long terminal repeat (LTR)) located immediately 5′ of the plastid modification cassette, (iii) a second recognition site located immediately 3′ of the plastid modification cassette, and (iv) aplastid localization sequence operably located 5′ of the first recognition site; and (c) a polynucleotide encoding a zinc-finger nuclease (ZFN) fused to a plastid transit peptide, wherein the ZFN comprises a zinc finger DNA-binding domain fused to a DNA-cleavage domain, thereby modifying the plastid genome of said plant cell.

In some embodiments, a method of modifying a plastid genome of a plant cell is provided, comprising, consisting essentially of, or consisting of: introducing into a plant cell introducing into a plant cell (a) a polynucleotide encoding a reverse transcriptase (RT) polypeptide fused to a plastid transit peptide; (b) a recombinant nucleic acid comprising: (i) a plastid modification cassette, (ii) a first recognition site (i.e., long terminal repeat (LTR)) located immediately 5′ of the plastid modification cassette, (iii) a second recognition site located immediately 3′ of the plastid modification cassette, and (iv) a plastid localization sequence operably located 5′ of the first recognition site; and (c) a polynucleotide encoding a meganuclease fused to a plastid transit peptide, thereby modifying the plastid genome of said plant cell

In some embodiments, a method of modifying a plastid genome of a plant cell is provided, comprising, consisting essentially of, or consisting of introducing into a plant cell (a) a recombinant nucleic acid linked to a plastid localization sequence and comprising a polynucleotide encoding a reverse transcriptase polypeptide and a sequence-specific nuclease; and (b) a recombinant nucleic acid comprising (i) a plastid modification cassette, (ii) a first recognition site located immediately 5′ of the plastid modification cassette, (iii) a second recognition site located immediately 3′ of the plastid modification cassette, and (iv) a plastid localizing sequence operably located 5′ of the first recognition site, thereby modifying the plastid genome of the plant cell.

In some embodiments, a method of modifying a plastid genome of a plant cell is provided, comprising, consisting essentially of, or consisting of introducing into a plant cell (a) a recombinant nucleic acid linked to a plastid localization sequence and comprising a polynucleotide encoding a reverse transcriptase polypeptide and a Cas9 nuclease; (b) a guide nucleic acid linked to a plastid localization sequence; and (c) a recombinant nucleic acid comprising (i) a plastid modification cassette, (ii) a first recognition site located immediately 5′ of the plastid modification cassette, (iii) a second recognition site located immediately 3′ of the plastid modification cassette, and (iv) a plastid localizing sequence operably located 5′ of the first recognition site, thereby modifying the plastid genome of the plant cell.

In some embodiments, a method of modifying a plastid genome of a plant cell is provided, comprising, consisting essentially of, or consisting of introducing into a plant cell (a) a recombinant nucleic acid linked to a plastid localization sequence and comprising a polynucleotide encoding a reverse transcriptase polypeptide and a Cpf1 nuclease; (b) a guide (e.g., crRNA, crDNA) nucleic acid linked to a plastid localization sequence; and (c) a recombinant nucleic acid comprising (i) a plastid modification cassette, (ii) a first recognition site located immediately 5′ of the plastid modification cassette, (iii) a second recognition site located immediately 3′ of the plastid modification cassette, and (iv) a plastid localizing sequence operably located 5′ of the first recognition site, thereby modifying the plastid genome of the plant cell.

In some embodiments, a method of modifying a plastid genome of a plant cell is provided, comprising, consisting essentially of, or consisting of introducing into a plant cell (a) a recombinant nucleic acid linked to a plastid localization sequence and comprising a polynucleotide encoding a reverse transcriptase polypeptide and a meganuclease; and (b) a recombinant nucleic acid comprising (i) a plastid modification cassette, (ii) a first recognition site located immediately 5′ of the plastid modification cassette, (iii) a second recognition site located immediately 3′ of the plastid modification cassette, and (iv) a plastid localizing sequence operably located 5′ of the first recognition site, thereby modifying the plastid genome of the plant cell.

In some embodiments, a method of modifying a plastid genome of a plant cell is provided, comprising, consisting essentially of, or consisting of introducing into a plant cell (a) a recombinant nucleic acid linked to a plastid localization sequence and comprising a polynucleotide encoding a reverse transcriptase polypeptide and a transcription activator-like (TAL) effector nuclease (TALEN); and (b) a recombinant nucleic acid comprising (i) a plastid modification cassette, (ii) a first recognition site located immediately 5′ of the plastid modification cassette, (iii) a second recognition site located immediately 3′ of the plastid modification cassette, and (iv) a plastid localizing sequence operably located 5′ of the first recognition site, thereby modifying the plastid genome of the plant cell.

In some embodiments, a method of modifying a plastid genome of a plant cell is provided, comprising, consisting essentially of, or consisting of introducing into a plant cell (a) a recombinant nucleic acid linked to a plastid localization sequence and comprising a polynucleotide encoding a reverse transcriptase polypeptide and a zinc finger nuclease (ZFN); and (b) a recombinant nucleic acid comprising (i) a plastid modification cassette, (ii) a first recognition site located immediately 5′ of the plastid modification cassette, (iii) a second recognition site located immediately 3′ of the plastid modification cassette, and (iv) a plastid localizing sequence operably located 5′ of the first recognition site, thereby modifying the plastid genome of the plant cell.

In some embodiments, a method of modifying a plastid genome of a plant cell is provided, comprising, consisting essentially of, or consisting of introducing into a plant cell a polynucleotide encoding an ATP-dependent DNA ligase D (LigD) fused to a plastid transit peptide and a polynucleotide encoding a DNA-binding protein Ku (Ku) fused to a plastid transit peptide, thereby modifying the plastid genome of said plant cell. In some embodiments, a method of modifying a mitochondrial genome of a cell is provided, comprising, consisting essentially of, or consisting of introducing into a cell a polynucleotide encoding an ATP-dependent DNA ligase D (LigD) fused to a mitochondrial transit peptide and a polynucleotide encoding a DNA-binding protein Ku (Ku) fused to a mitochondrial transit peptide, thereby modifying the mitochondrial genome of said plant cell.

In further embodiments, a method of modifying a plastid genome of a plant cell is provided, comprising, consisting essentially of, or consisting of introducing into a plant cell a polynucleotide encoding an ATP-dependent DNA ligase D (LigD) fused to a plastid transit peptide, a polynucleotide encoding a DNA-binding protein Ku (Ku) fused to a plastid transit peptide, and a polynucleotide encoding a sequence-specific nuclease fused to a plastid transit peptide, thereby modifying the plastid genome of said plant. In some embodiments, a method of modifying a mitochondrial genome of a cell is provided, comprising, consisting essentially of, or consisting of introducing into a cell a polynucleotide encoding an ATP-dependent DNA ligase D (LigD) fused to a mitochondrial transit peptide, a polynucleotide encoding a DNA-binding protein Ku (Ku) fused to a mitochondrial transit peptide and a polynucleotide encoding a sequence-specific nuclease fused to a mitochondrial transit peptide, thereby modifying the mitochondrial genome of the cell.

In some embodiments, a method of modifying a plastid genome of a plant cell is provided, comprising, consisting essentially of, or consisting of introducing into a plant cell a polynucleotide encoding an ATP-dependent DNA ligase D (LigD) fused to a plastid transit peptide, a polynucleotide encoding a DNA-binding protein Ku (Ku) fused to a plastid transit peptide, a polynucleotide encoding a Cas9 nuclease fused to a plastid transit peptide and a guide nucleic acid linked to a plastid localization sequence, thereby modifying the plastid genome of said plant. In some embodiments, a method of modifying a mitochondrial genome of a cell is provided, comprising, consisting essentially of, or consisting of introducing into a cell a polynucleotide encoding an ATP-dependent DNA ligase D (LigD) fused to a mitochondrial transit peptide, a polynucleotide encoding a DNA-binding protein Ku (Ku) fused to a mitochondrial transit peptide, a polynucleotide encoding a Cas9 nuclease fused to a mitochondrial transit peptide and a guide nucleic acid linked to a mitochondrial localization sequence mitochondrial transit peptide, thereby modifying the mitochondrial genome of the cell.

In some embodiments, a method of modifying a plastid genome of a plant cell is provided, comprising, consisting essentially of, or consisting of introducing into a plant cell a polynucleotide encoding an ATP-dependent DNA ligase D (LigD) fused to a plastid transit peptide, a polynucleotide encoding a DNA-binding protein Ku (Ku) fused to a plastid transit peptide, a polynucleotide encoding a Cpf1 nuclease fused to a plastid transit peptide and a guide nucleic acid (e.g., crRNA, crDNA) linked to a plastid localization sequence, thereby modifying the plastid genome of said plant. In some embodiments, a method of modifying a mitochondrial genome of a cell is provided, comprising, consisting essentially of, or consisting of introducing into a cell a polynucleotide encoding an ATP-dependent DNA ligase D (LigD) fused to a mitochondrial transit peptide, a polynucleotide encoding a DNA-binding protein Ku (Ku) fused to a mitochondrial transit peptide, a polynucleotide encoding a Cpf1 nuclease fused to a mitochondrial transit peptide and a guide nucleic acid (e.g., crRNA, crDNA) linked to a mitochondrial localization sequence mitochondrial transit peptide, thereby modifying the mitochondrial genome of the cell.

In some embodiments, a method of modifying a plastid genome of a plant cell is provided, comprising, consisting essentially of, or consisting of introducing into a plant cell a polynucleotide encoding an ATP-dependent DNA ligase D (LigD) fused to a plastid transit peptide, a polynucleotide encoding a DNA-binding protein Ku (Ku) fused to a plastid transit peptide, and a polynucleotide encoding a transcription activator-like (TAL) effector nuclease (TALEN) fused to a plastid transit peptide, thereby modifying the plastid genome of said plant. In some embodiments, a method of modifying a mitochondrial genome of a cell is provided, comprising, consisting essentially of, or consisting of introducing into a cell a polynucleotide encoding an ATP-dependent DNA ligase D (LigD) fused to a mitochondrial transit peptide, a polynucleotide encoding a DNA-binding protein Ku (Ku) fused to a mitochondrial transit peptide and a polynucleotide encoding a transcription activator-like (TAL) effector nuclease (TALEN) fused to a mitochondrial transit peptide, thereby modifying the mitochondrial genome of the cell.

In some embodiments, a method of modifying a plastid genome of a plant cell is provided, comprising, consisting essentially of, or consisting of introducing into a plant cell a polynucleotide encoding an ATP-dependent DNA ligase D (LigD) fused to a plastid transit peptide, a polynucleotide encoding a DNA-binding protein Ku (Ku) fused to a plastid transit peptide, and a polynucleotide encoding zinc-finger nuclease (ZFN) fused to a plastid transit peptide, thereby modifying the plastid genome of said plant. In some embodiments, a method of modifying a mitochondrial genome of a cell is provided, comprising, consisting essentially of, or consisting of introducing into a cell a polynucleotide encoding an ATP-dependent DNA ligase D (LigD) fused to a mitochondrial transit peptide, a polynucleotide encoding a DNA-binding protein Ku (Ku) fused to a mitochondrial transit peptide and a polynucleotide encoding a zinc-finger nuclease (ZFN) fused to a mitochondrial transit peptide, thereby modifying the mitochondrial genome of the cell.

In other embodiments, a method of modifying a plastid genome of a plant cell is provided, comprising, consisting essentially of, or consisting of introducing into a plant cell a polynucleotide encoding an ATP-dependent DNA ligase D (LigD) fused to a plastid transit peptide, a polynucleotide encoding a DNA-binding protein Ku (Ku) fused to a plastid transit peptide, and a polynucleotide encoding a meganuclease fused to a plastid transit peptide thereby modifying the plastid genome of said plant. In some embodiments, a method of modifying a mitochondrial genome of a cell is provided, comprising, consisting essentially of, or consisting of introducing into a cella polynucleotide encoding an ATP-dependent DNA ligase D (LigD) fused to a mitochondrial transit peptide, a polynucleotide encoding a DNA-binding protein Ku (Ku) fused to a mitochondrial transit peptide and a polynucleotide encoding a meganuclease fused to a mitochondrial transit peptide, thereby modifying the mitochondrial genome of the cell.

In embodiments in which a mitochondrial genome is to be modified, the cell may be any eukaryotic cell (e.g., a plant, a fungus, an animal, and the like)

In some embodiments, the present invention further provides a method of expressing a polynucleotide sequence of interest (POI) in a plastid, comprising, consisting essentially of, or consisting of introducing into a plant cell: (a) a polynucleotide encoding a reverse transcriptase polypeptide fused to a plastid transit peptide and a sequence-specific nuclease fused to a plastid transit peptide, or a polynucleotide linked to a plastid localization sequence and encoding a reverse transcriptase polypeptide and a sequence-specific nuclease; and (b) a recombinant nucleic acid comprising (i) a plastid modification cassette, (ii) a first recognition site located immediately 5′ of the plastid modification cassette, (iii) a second recognition site located immediately 3′ of the plastid modification cassette, and (iv) a plastid localization sequence operably located 5′ of the first recognition site, wherein said plastid modification cassette comprises a POI, thereby expressing the POI in a plastid.

In some embodiments, the present invention further provides a method of expressing a polynucleotide sequence of interest (POI) in a plastid, comprising, consisting essentially of, or consisting of introducing into a plant cell: (a) a polynucleotide encoding a reverse transcriptase polypeptide fused to a plastid transit peptide and a Cas9 nuclease fused to a plastid transit peptide, or a polynucleotide linked to a plastid localization sequence and encoding a reverse transcriptase polypeptide and a Cas9 nuclease; (b) a guide nucleic acid linked to a plastid localization sequence; and (c) a recombinant nucleic acid comprising (i) a plastid modification cassette, (ii) a first recognition site located immediately 5′ of the plastid modification cassette, (iii) a second recognition site located immediately 3′ of the plastid modification cassette, and (iv) a plastid localization sequence operably located 5′ of the first recognition site, wherein said plastid modification cassette comprises a POI, thereby expressing the POI in a plastid. In some embodiments, the polynucleotide linked to a plastid localization sequence and encoding a reverse transcriptase polypeptide and a Cas9 nuclease may further comprise the guide nucleic acid.

In some embodiments, the present invention further provides a method of expressing a polynucleotide sequence of interest (POI) in a plastid, comprising, consisting essentially of, or consisting of introducing into a plant cell: (a) a polynucleotide encoding a reverse transcriptase polypeptide fused to a plastid transit peptide and a Cpf1 nuclease fused to a plastid transit peptide, or a polynucleotide linked to a plastid localization sequence and encoding a reverse transcriptase polypeptide and a Cpf1 nuclease; (b) a guide nucleic acid (e.g., crRNA, crDNA) linked to a plastid localization sequence; and (c) a recombinant nucleic acid comprising (i) a plastid modification cassette, (ii) a first recognition site located immediately 5′ of the plastid modification cassette, (iii) a second recognition site located immediately 3′ of the plastid modification cassette, and (iv) a plastid localization sequence operably located 5′ of the first recognition site, wherein said plastid modification cassette comprises a POI, thereby expressing the POI in a plastid. In some embodiments, the polynucleotide linked to a plastid localization sequence and encoding a reverse transcriptase polypeptide and a Cas9 nuclease may further comprise the guide nucleic acid.

In some embodiments, the present invention further provides a method of expressing a polynucleotide sequence of interest (POI) in a plastid, comprising, consisting essentially of, or consisting of introducing into a plant cell: (a) a polynucleotide encoding a reverse transcriptase polypeptide fused to a plastid transit peptide and a transcription activator-like (TAL) effector nuclease (TALEN) fused to a plastid transit peptide, or a polynucleotide linked to a plastid localization sequence and encoding a reverse transcriptase polypeptide and a transcription activator-like (TAL) effector nuclease; and (b) a recombinant nucleic acid comprising (i) a plastid modification cassette, (ii) a first recognition site located immediately 5′ of the plastid modification cassette, (iii) a second recognition site located immediately 3′ of the plastid modification cassette, and (iv) a plastid localization sequence operably located 5′ of the first recognition site, wherein said plastid modification cassette comprises a POI, thereby expressing the POI in a plastid.

In some embodiments, the present invention further provides a method of expressing a polynucleotide sequence of interest (POI) in a plastid, comprising, consisting essentially of, or consisting of introducing into a plant cell: (a) a polynucleotide encoding a reverse transcriptase polypeptide fused to a plastid transit peptide and a zinc-finger nuclease (ZFN) fused to a plastid transit peptide, or a polynucleotide linked to a plastid localization sequence and encoding a reverse transcriptase polypeptide and a zinc-finger nuclease (ZFN); and (b) a recombinant nucleic acid comprising (i) a plastid modification cassette, (ii) a first recognition site located immediately 5′ of the plastid modification cassette, (iii) a second recognition site located immediately 3′ of the plastid modification cassette, and (iv) a plastid localization sequence operably located 5′ of the first recognition site, wherein said plastid modification cassette comprises a POI, thereby expressing the POI in a plastid.

In some embodiments, the present invention further provides a method of expressing a polynucleotide sequence of interest (POI) in a plastid, comprising, consisting essentially of, or consisting of introducing into a plant cell: (a) a polynucleotide encoding a reverse transcriptase polypeptide fused to a plastid transit peptide and a meganuclease fused to a plastid transit peptide, or a polynucleotide linked to a plastid localization sequence and encoding a reverse transcriptase polypeptide and a meganuclease; and (b) a recombinant nucleic acid comprising (i) a plastid modification cassette, (ii) a first recognition site located immediately 5′ of the plastid modification cassette, (iii) a second recognition site located immediately 3′ of the plastid modification cassette, and (iv) a plastid localization sequence operably located 5′ of the first recognition site, wherein said plastid modification cassette comprises a POI, thereby expressing the POI in a plastid.

In some embodiments, the present invention further provides a method of transforming a plastid genome, comprising, consisting essentially of, or consisting of: introducing into a plant cell: (a) a polynucleotide encoding a reverse transcriptase polypeptide fused to a plastid transit peptide and a sequence-specific nuclease fused to a plastid transit peptide, or a polynucleotide linked to a plastid localization sequence and encoding a reverse transcriptase polypeptide and a sequence-specific nuclease; and (b) a recombinant nucleic acid comprising (i) a plastid modification cassette, (ii) a first recognition site located immediately 5′ of the plastid modification cassette, (iii) a second recognition site located immediately 3′ of the plastid modification cassette, and (iv) a plastid localization sequence operably located 5′ of the first recognition site, wherein said plastid modification cassette comprises a POI, thereby transforming said plastid genome.

In some embodiments, the present invention further provides a method of transforming a plastid genome, comprising, consisting essentially of, or consisting of; introducing into a plant cell: (a) a polynucleotide encoding a reverse transcriptase polypeptide fused to a plastid transit peptide and a Cas9 nuclease fused to a plastid transit peptide, or a polynucleotide linked to a plastid localization sequence and encoding a reverse transcriptase polypeptide and a Cas9 nuclease; (b) a guide nucleic acid linked to a plastid localization sequence; and (c) a recombinant nucleic acid comprising (i) a plastid modification cassette, (ii) a first recognition site located immediately 5′ of the plastid modification cassette, (iii) a second recognition site located immediately 3′ of the plastid modification cassette, and (iv) a plastid localization sequence operably located 5′ of the first recognition site, wherein said plastid modification cassette comprises a POI, thereby transforming said plastid genome.

In some embodiments, the present invention further provides a method of transforming a plastid genome, comprising, consisting essentially of, or consisting of: introducing into a plant cell: (a) a polynucleotide encoding a reverse transcriptase polypeptide fused to a plastid transit peptide and a Cpf1 nuclease fused to a plastid transit peptide, or a polynucleotide linked to a plastid localization sequence and encoding a reverse transcriptase polypeptide and a Cpf1 nuclease; (b) a guide nucleic acid (e.g., crRNA, crDNA) linked to a plastid localization sequence; and (c) a recombinant nucleic acid comprising (i) a plastid modification cassette, (ii) a first recognition site located immediately 5′ of the plastid modification cassette, (iii) a second recognition site located immediately 3′ of the plastid modification cassette, and (iv) a plastid localization sequence operably located 5′ of the first recognition site, wherein said plastid modification cassette comprises a POI, thereby transforming said plastid genome.

In some embodiments, the present invention further provides a method of transforming a plastid genome, comprising, consisting essentially of, or consisting of: introducing into a plant cell: (a) a polynucleotide encoding a reverse transcriptase polypeptide fused to a plastid transit peptide and a s transcription activator-like (TAL) effector nuclease fused to a plastid transit peptide, or a polynucleotide linked to a plastid localization sequence and encoding a reverse transcriptase polypeptide and a transcription activator-like (TAL) effector nuclease; and (b) a recombinant nucleic acid comprising (i) a plastid modification cassette, (ii) a first recognition site located immediately 5′ of the plastid modification cassette, (iii) a second recognition site located immediately 3′ of the plastid modification cassette, and (iv) a plastid localization sequence operably located 5′ of the first recognition site, wherein said plastid modification cassette comprises a POI, thereby transforming said plastid genome.

In some embodiments, the present invention further provides a method of transforming a plastid genome, comprising, consisting essentially of, or consisting of: introducing into a plant cell: (a) a polynucleotide encoding a reverse transcriptase polypeptide fused to a plastid transit peptide and a zinc-finger nuclease (ZFN) fused to a plastid transit peptide, or a polynucleotide linked to a plastid localization sequence and encoding a reverse transcriptase polypeptide and a zinc-finger nuclease (ZFN); and (b) a recombinant nucleic acid comprising (i) a plastid modification cassette, (ii) a first recognition site located immediately 5′ of the plastid modification cassette, (iii) a second recognition site located immediately 3′ of the plastid modification cassette, and (iv) a plastid localization sequence operably located 5′ of the first recognition site, wherein said plastid modification cassette comprises a POI, thereby transforming said plastid genome.

In some embodiments, the present invention further provides a method of transforming a plastid genome, comprising, consisting essentially of, or consisting of: introducing into a plant cell: (a) a polynucleotide encoding a reverse transcriptase polypeptide fused to a plastid transit peptide and a meganuclease fused to a plastid transit peptide, or a polynucleotide linked to a plastid localization sequence and encoding a reverse transcriptase polypeptide and a meganuclease; and (b) a recombinant nucleic acid comprising (i) a plastid modification cassette, (ii) a first recognition site located immediately 5′ of the plastid modification cassette, (iii) a second recognition site located immediately 3′ of the plastid modification cassette, and (iv) a plastid localization sequence operably located 5′ of the first recognition site, wherein said plastid modification cassette comprises a POI, thereby transforming said plastid genome.

The present invention further provides methods of producing plants and plant cells having modified plastid genomes. Thus, in some embodiments, a method of producing a plant cell having a modified plastid genome is provided, comprising, consisting essentially of, or consisting of introducing into a plant cell (a) a polynucleotide encoding a reverse transcriptase (RT) polypeptide fused to a plastid transit peptide; (b) a recombinant nucleic acid comprising: (i) a plastid modification cassette, (ii) a first recognition site located immediately 5′ of the plastid modification cassette, (iii) a second recognition site located immediately 3′ of the plastid modification cassette, and (iv) a plastid localization sequence operably located 5′ of the first recognition site; and (c) a polynucleotide encoding a sequence-specific nuclease fused to a plastid transit peptide, thereby producing a plant cell having a modified plastid genome.

In some embodiments a method of producing a plant cell having a modified plastid genome is provided, comprising, consisting essentially of, or consisting of introducing into a plant cell (a) a polynucleotide encoding a reverse transcriptase (RT) polypeptide fused to a plastid transit peptide; (b) a recombinant nucleic acid comprising: (i) a plastid modification cassette, (ii) a first recognition site located immediately 5′ of the plastid modification cassette, (iii) a second recognition site located immediately 3′ of the plastid modification cassette, and (iv) a plastid localization sequence operably located 5′ of the first recognition site; (c) a polynucleotide encoding a Cas9 nuclease fused to a plastid transit peptide; and (d) a guide nucleic acid linked to a plastid localization sequence, thereby producing a plant cell having a modified plastid genome.

In some embodiments a method of producing a plant cell having a modified plastid genome is provided, comprising, consisting essentially of, or consisting of introducing into a plant cell (a) a polynucleotide encoding a reverse transcriptase (RT) polypeptide fused to a plastid transit peptide; (b) a recombinant nucleic acid comprising: (i) a plastid modification cassette, (ii) a first recognition site located immediately 5′ of the plastid modification cassette, (iii) a second recognition site located immediately 3′ of the plastid modification cassette, and (iv) a plastid localization sequence operably located 5′ of the first recognition site; (c) a polynucleotide encoding a Cpf1 nuclease fused to a plastid transit peptide; and (d) a guide nucleic acid (e.g., crRNA, crDNA) linked to a plastid localization sequence, thereby producing a plant cell having a modified plastid genome.

In some embodiments, a method of producing a plant cell having a modified plastid genome is provided, comprising, consisting essentially of, or consisting of introducing into a plant cell (a) a polynucleotide encoding a reverse transcriptase (RT) polypeptide fused to a plastid transit peptide; (b) a recombinant nucleic acid comprising: (i) a plastid modification cassette, (ii) a first recognition site located immediately 5′ of the plastid modification cassette, (iii) a second recognition site located immediately 3′ of the plastid modification cassette, and (iv) a plastid localization sequence operably located 5′ of the first recognition site; and (c) a polynucleotide encoding a transcription activator-like (TAL) effector nuclease (TALEN) fused to a plastid transit peptide, thereby producing a plant cell having a modified plastid genome.

In some embodiments, a method of producing a plant cell having a modified plastid genome is provided, comprising, consisting essentially of, or consisting of introducing into a plant cell (a) a polynucleotide encoding a reverse transcriptase (RT) polypeptide fused to a plastid transit peptide; (b) a recombinant nucleic acid comprising: (i) a plastid modification cassette, (ii) a first recognition site located immediately 5′ of the plastid modification cassette, (iii) a second recognition site located immediately 3′ of the plastid modification cassette, and (iv) a plastid localization sequence operably located 5′ of the first recognition site; and (c) a polynucleotide encoding a zinc-finger nuclease (ZFN), fused to a plastid transit peptide, thereby producing a plant cell having a modified plastid genome.

In some embodiments, a method of producing a plant cell having a modified plastid genome is provided, comprising, consisting essentially of, or consisting of introducing into a plant cell (a) a polynucleotide encoding a reverse transcriptase (RT) polypeptide fused to a plastid transit peptide; (b) a recombinant nucleic acid comprising: (i) a plastid modification cassette, (ii) a first recognition site located immediately 5′ of the plastid modification cassette, (iii) a second recognition site located immediately 3′ of the plastid modification cassette, and (iv) a plastid localization sequence operably located 5′ of the first recognition site; and (c) a polynucleotide encoding a meganuclease fused to a plastid transit peptide, thereby producing a plant cell having a modified plastid genome.

In some embodiments, a method of producing a plant cell having a modified plastid genome is provided, comprising, consisting essentially of, or consisting of introducing into a plant cell (a) a recombinant nucleic acid linked to a plastid localization sequence and comprising a polynucleotide encoding a reverse transcriptase polypeptide and a sequence-specific nuclease; and(b) a recombinant nucleic acid comprising: (i) a plastid modification cassette, (ii) a first recognition site located immediately 5′ of the plastid modification cassette, (iii) a second recognition site located immediately 3′ of the plastid modification cassette, and (iv) a plastid localizing sequence operably located 5′ of the first recognition site, thereby modifying the plastid genome of the plant cell.

In some embodiments, a method of producing a plant cell having a modified plastid genome is provided, comprising, consisting essentially of, or consisting of introducing into a plant cell (a) a recombinant nucleic acid linked to a plastid localization sequence and comprising a polynucleotide encoding a reverse transcriptase polypeptide and a Cas9 nuclease; (b) a guide nucleic acid linked to a plastid localization sequence; and (c) a recombinant nucleic acid comprising: (i) a plastid modification cassette, (ii) a first recognition site located immediately 5′ of the plastid modification cassette, (iii) a second recognition site located immediately 3′ of the plastid modification cassette, and (iv) a plastid localizing sequence operably located 5′ of the first recognition site, thereby modifying the plastid genome of the plant cell.

In some embodiments, a method of producing a plant cell having a modified plastid genome is provided, comprising, consisting essentially of, or consisting of introducing into a plant cell (a) a recombinant nucleic acid linked to a plastid localization sequence and comprising a polynucleotide encoding a reverse transcriptase polypeptide and a Cpf1 nuclease; (b) a guide nucleic acid (e.g., crRNA, crDNA) linked to a plastid localization sequence; and (c) a recombinant nucleic acid comprising: (i) a plastid modification cassette, (ii) a first recognition site located immediately 5′ of the plastid modification cassette, (iii) a second recognition site located immediately 3′ of the plastid modification cassette, and (iv) a plastid localizing sequence operably located 5′ of the first recognition site, thereby modifying the plastid genome of the plant cell.

In further embodiments, a method of producing a plant cell having a modified plastid genome is provided, comprising, consisting essentially of, or consisting of introducing into a plant cell (a) a recombinant nucleic acid linked to a plastid localization sequence and comprising a polynucleotide encoding a reverse transcriptase polypeptide and a transcription activator-like (TAL) effector nuclease (TALEN); and (b) a recombinant nucleic acid comprising: (i) a plastid modification cassette, (ii) a first recognition site located immediately 5′ of the plastid modification cassette, (iii) a second recognition site located immediately 3′ of the plastid modification cassette, and (iv) a plastid localizing sequence operably located 5′ of the first recognition site, thereby modifying the plastid genome of the plant cell.

In additional embodiments, a method of producing a plant cell having a modified plastid genome is provided, comprising, consisting essentially of, or consisting of introducing into a plant cell (a) a recombinant nucleic acid linked to a plastid localization sequence and comprising a polynucleotide encoding a reverse transcriptase polypeptide and a zinc-finger nuclease (ZFN); and (b) a recombinant nucleic acid comprising: (i) a plastid modification cassette, (ii) a first recognition site located immediately 5′ of the plastid modification cassette, (iii) a second recognition site located immediately 3′ of the plastid modification cassette, and (iv) a plastid localizing sequence operably located 5′ of the first recognition site, thereby modifying the plastid genome of the plant cell.

In some embodiments, a method of producing a plant cell having a modified plastid genome is provided, comprising, consisting essentially of, or consisting of introducing into a plant cell (a) a recombinant nucleic acid linked to a plastid localization sequence and comprising a polynucleotide encoding a reverse transcriptase polypeptide and a meganuclease; and (b) a recombinant nucleic acid comprising: (i) a plastid modification cassette, (ii) a first recognition site located immediately 5′ of the plastid modification cassette, (iii) a second recognition site located immediately 3′ of the plastid modification cassette, and (iv) a plastid localizing sequence operably located 5′ of the first recognition site, thereby modifying the plastid genome of the plant cell.

As understood in the art, the various components that are introduced into the plant (e.g., polynucleotides, recombinant nucleic acids) may be introduced singly or together in any combination with individual or shared regulatory elements. Thus, for example, a polynucleotide encoding a Cas9 polypeptide fused to a plastid transit peptide and a polynucleotide encoding a RT fused to a plastid transit peptide may be introduced individually or in the same construct, and the guide nucleic acid and the recombinant nucleic acid can be introduced individually or in the same construct, wherein each is linked to a separate plastid localization sequence that may be the same or different plastid localization sequences. Thus, in any of the above described embodiments, the polynucleotide encoding a sequence-specific nuclease, the polynucleotide encoding a RT polypeptide, the polynucleotide encoding LigD, and/or the polynucleotide encoding Ku may be operably linked to one or more promoters and optionally, operably linked to one or more terminators, wherein the promoters may be the same or different and the terminators may be the same or different. Further, in any of the above described embodiments, a polynucleotide encoding a sequence specific nuclease (e.g., Cpfl nuclease, Cas9 nuclease, TALEN, ZFN and meganuclease) fused to a plastid transit peptide, a polynucleotide encoding an ATP-dependent DNA ligase D (LigD) fused to a plastid transit peptide and a polynucleotide encoding a DNA-binding protein Ku (Ku) fused to a plastid transit peptide may be introduced individually or in the same construct or in any combination thereof.

In some embodiments, a first recognition sequence can comprise, consist of, or consist essentially of, 5′ to 3′, a viral or retrotransposon R sequence, a viral or retrotransposon derived 5′ untranslated region (5′ UTR) and a primer binding site (PBS), and a second recognition sequence comprises a polypurine tract (PPT), a viral or retrotransposon derived 3′ untranslated region (3′ UTR), and a viral or retrotransposon R sequence, wherein the viral or retrotransposon R sequence of the first recognition sequence and the viral or retrotransposon R sequence of the second recognition sequence are identical. A viral or retrotransposon R sequence, a viral or retrotransposon derived 5′UTR or 3′ UTR regions, PBS, and/or PPT useful with this invention can be any known or later identified viral or retrotransposon R sequence, viral or retrotransposon derived 5′UTR or 3′ UTR, PBS, and/or PPT. Exemplary viral R sequences include, but is not limited to, those from

Moloney Murine Leukemia Virus (MMLV) (Genbank:
NC_001501.1)
(GCGCCAGTCCTCCGATTGACTGAGTCGCCCGGGTACCCGTGTATCCAAT
AAACCCTCTTGCAGTTGCA (SEQ ID NO: 1))
and/or
Human Immunodeficiency Virus 1 (HIV-1) (Genbank:
AF033819.3)
(GGTCTCTCTGGTTAGACCAGATCTGAGCCTGGGAGCTCTCTGGCTAACT
AGGGAACCCACTGCTTAAGCCTCAATAAAGCTTGCCTTGAGTGCTTC
(SEQ ID NO: 2)).

Exemplary viral derived 5′ UTR regions include, but are not limited to, those from

MMLV (Genbank: NC_001501.1)
(TCCGACTTGTGGTCTCGCTGTTCCTTGGGAGGGTCTCCTCTG
AGTGATTGACTACCCGTCAGCGGGGGTCTTTCATT (SEQ ID NO: 3))
and/or
HIV-1 (Genbank: AF033819.3)
(AAGTAGTGTGTGCCCGTCTGTTGTGTGACTCTGGTAACTAGAGATCCCT
CAGACCCTTTTAGTCAGTGTGGAAAATCTCTAGCAG (SEQ ID
NO: 4)).

A primer binding site (PBS) can include, but is not limited to, those from

MMLV (Genbank: NC_001501.1)
(TGGGGGGCGTTCCGAGAA (SEQ ID NO: 5))
and/or
HIV-1 (Genbank: AF033819.3)
(TGGCGCCCGAACAGGG AC (SEQ ID NO: 6)).

A PPT useful with the invention can include, but is not limited to, those from a PPT from

MMLV (Genbank: NC_001501.1)
(AAAAAGGGGGGAATGAAA (SEQ ID NO: 7))
and or
HIV-1 (Genbank: AF033819.3)
(AAAAGAAAAGGGGGGA (SEQ ID NO: 8)).

Exemplary 3′ UTR regions can include those from

MMLV (Genbank: NC_001501.1)
(GACCCCACCTGTAGGTTTGGCAAGCTAGCTTAAGTAACGCCATTTTGCA
AGGCATGGAAAAATACATAACTGAGAATAGAGAAGTTCAGATCAAGGTCA
GGAACAGATGGAACAGCTGAATATGGGCCAAACAGGATATCTGTGGTAAG
CAGTTCCTGCCCCGGCTCAGGGCCAAGAACAGATGGAACAGCTGAATATG
GGCCAAACAGGATATCTGTGGTAAGCAGTTCCTGCCCCGGCTCAGGGCCA
AGAACAGATGGTCCCCAGATGCGGTCCAGCCCTCAGCAGTTTCTAGAGAA
CCATCAGATGTTTCCAGGGTGCCCCAAGGACCTGAAATGACCCTGTGCCT
TATTTGAACTAACCAATCAGTTCGCTTCTCGCTTCTGTTCGCGCGCTTCT
GCTCCCCGAGCTCAATAAAAGAGCCCACAACCCCTCACTCGGG (SEQ
ID NO: 9))
and/or
HIV-1 (Genbank: AF033819.3)
(CTGGAAGGGCTAATTCACTCCCAAAGAAGACAAGATATCCTTGATCTGT
GGATCTACCACACACAAGGCTACTTCCCTGATTAGCAGAACTACACACCA
GGGCCAGGGGTCAGATATCCACTGACCTTTGGATGGTGCTACAAGCTAGT
ACCAGTTGAGCCAGATAAGATAGAAGAGGCCAATAAAGGAGAGAACACCA
GCTTGTTACACCCTGTGAGCCTGCATGGGATGGATGACCCGGAGAGAGAA
GTGTTAGAGTGGAGGTTTGACAGCCGCCTAGCATTTCATCACGTGGCCCG
AGAGCTGCATCCGGAGTACTTCAAGAACTGCTGACATCGAGCTTGCTACA
AGGGACTTTCCGCTGGGGACTTTCCAGGGAGGCGTGGCCTGGGCGGGACT
GGGGAGTGGCGAGCCCTCAGATCCTGCATATAAGCAGCTGCTTTTTGCCT
GTACTG (SEQ ID NO: 10)).

Additional, exemplary R sequences, 5′ UTR regions, PBS, PPT and/or 3′UTR regions can be derived from CaMV (Cauliflower Mosaic Virus).

A plastid modification cassette of the invention may be designed to modify the plastid genome in any number of ways. Thus, for example, the plastid modification cassette may be designed so that it can be used in combination with a reverse transcriptase polypeptide to delete one or more nucleotides or an entire nucleic acid region (transcribed and untranscribed regions), alter (inhibiting and/or activating) the expression of an endogenous polynucleotide, introduce one or more point mutations, introduce a synthetic promoter, introduce a regulatory RNA, introduce a polynucleotide to be expressed from an endogenous operon (e.g., no promoter or other regulatory sequence introduced), introducing a gene expression construct and/or introducing an operon expression construct, and the like.

In some embodiments, a plastid modification cassette comprises, consists of, or consists essentially of a first homology arm and a second homology arm, optionally wherein the plastid modification cassette further comprises, consists of, or consists essentially of an intervening synthetic nucleotide sequence (i.e., intervening sequence) up to about 10 kb in size located between the first and second homology arms. Thus, depending on whether, for example, a nucleic acid is to be introduced or a deletion is to be introduced, the modification cassette may or may not comprise an intervening sequence between the first homology arm and the second homology arm. The first and second homology arms are homologous to regions of the plastid DNA flanking the site in the plastid genome that is to be modified. In some embodiments, the homology arms are species specific, e.g., the plastid modification cassette is designed to work in a specific species or variety.

As used herein, an “intervening synthetic nucleotide sequence” can be any synthetic nucleotide sequence useful for modifying a plastid genome. Such sequences can be designed to introduce one or more point mutations, introduce one or more synthetic promoter, introduce one or more regulatory nucleic acids, introduce one or more polynucleotides of interest, introduce one or more gene expression constructs and/or introduce one or more operon expression constructs, or any combination thereof.

Many different modifications can be made to a plastid genome using the methods described herein. For example, a plastid modification cassette can be used to delete a gene or part thereof. In this example, no intervening sequence is located between the homology arms in the plastid modification cassette. In a further example, one or more point mutations can be introduced by incorporating one or more mutated base pairs into the intervening sequence. An additional embodiment comprises introducing a synthetic promoter into the plastid genome, wherein the intervening sequence comprises a synthetic promoter, which may replace an endogenous promoter. In a further embodiment, the method can comprise introducing a polynucleotide encoding a regulatory RNA, wherein the intervening sequence comprises, for example, a promoter/regulatory RNA/terminator that is to be introduced into the plastid genome. In a still further example, a polynucleotide of interest (POI) can be expressed using an endogenous operon by designing the intervening sequence to comprise the POI without any promoter, terminator or other regulatory sequence. Alternatively, as provided herein, an intervening sequence can comprise an expression construct including a promoter/POI/terminator, thereby expressing a POI in the plastid independently of any endogenous controls. Furthermore, operons may be introduced on an intervening sequence (e.g., a promoter/POI A/POI B/POI C (etc.)/terminator) between the homology arms.

In some embodiments, a modification to a plastid genome can include, for example, the introduction of an indel (insertion or deletion) to disrupt expression of a target nucleic acid. Such modifications can be carried out by introducing a polynucleotide encoding sequence-specific nuclease as described herein, (e.g., Cpf1 nuclease, a TALEN, a ZFN, a meganuclease and/or a Cas9 polypeptide and a guide nucleic acid (a crRNA/crDNA or a crRNA/crDNA and tracrRNA/tracrDNA) with or without the introduction of a polypeptide encoding LigD and Ku.

A “guide nucleic acid” of the invention comprises a recombinant CRISPR array (crRNA/crDNA) and a recombinant trans-activating CRISPR (tracr) nucleic acid, or a CRISPR array only in the case of Cpfl. The recombinant CRISPR array comprises at least one CRISPR spacer-repeat nucleic acid comprising: (a) a spacer sequence comprising a 5′ end and a 3′ end; and (b) a Type II CRISPR (Cas9) or Type V (Cpfl) repeat sequence, comprising a 5′ end and a 3′ end, wherein the spacer sequence is linked at its 3′end to the 5′ end of the repeat. In some embodiments, a recombinant CRISPR array and a recombinant tracr nucleic acid can be fused to form a chimeric or single guide nucleic acid (sgDNA, sgRNA).

Plastid transit peptides facilitate the targeting and translocation of cytosolically synthesized polypeptides into plastids. A plastid transit peptide useful with this invention can be any known or later identified plastid transit peptide sequence (see, e.g., Lee et al. The Plant Cell, 20(6), 1603-1622 (2008)). Exemplary plastid transit peptides include the transit peptide from ribulose-1,5-bisphosphate carboxylase/oxygenase small subunit (rbcS) (e.g., from C. reinhardtii, Arabidopsis, and/or tobacco), Arabidopsis presequence protease1 (AT3G19170), Chlamydomonas reinhardtii-(Stroma-targeting cTPs: photosystem I (PSI) subunits P28, P30, P35 and P37, respectively), chlorophyll a/b binding protein (e.g., from C. reinhardtii), C. reinhardtii—ATPase-γ, biotin carboxyl carrier protein (e.g., from C. reinhardtii and/or Arabidopsis), ferredoxin-dependent glutamate synthase 2 and/or protochlorophyllide oxidoreductase A (See, Table 1). Thus, in some embodiments, a polypeptide of the invention (e.g., a Cpf1 polypeptide, a Cas9 polypeptide, a reverse transcriptase (RT) polypeptide, an ATP-dependent DNA ligase D, and/or a DNA-binding protein Ku) may be fused to a plastid transit peptide to target and translocate said polypeptide into a plastid. Generally, a transit peptide is fused to the N-terminal end of the polypeptide that is to be translocated.

TABLE 1
Amino acid sequences of representative plastid transit peptides.

| Source | Sequence |
|---|---|
| Rubisco small | MASSVLSSAAVATRSNVAQANMVAPFTGLKSAASFPVS |
| subunit (tobacco) | RKQNLDITSIASNGGRVQC (SEQ ID NO: 11) (NCBI |
| | Accession No: pfam12338) |
| Arabidopsis | MLRTVSCLASRSSSSLFFRFFRQFPRSYMSLTSSTAALRV |
| presequence | PSRNLRRISSPSVAGRRLLLRRGLRIPSAAVRSVNGQFSR |
| protease1 | LSVRA (SEQ ID NO: 12) (GenBank Accession No: |
| (AT3G19170) | NP_001189932) |
| Chlamydomonas | MALVARPVLSARVAASRPRVAARKAVRVSAKYGEN |
| reinhardtii- | (SEQ ID NO: 13) (NCBI Accession No: pfam03244) |
| (Stroma-targeting | MQALSSRVNIAAKPQRAQRLVVRAEEVKA (SEQ ID |
| cTPs: photosystem | NO: 14) (GenBank Accession No: XP_001702611) |
| I (PSI) subunits | MQTLASRPSLRASARVAPRRAPRVAVVTKAALDPQ |
| P28, P30, P35 and | (SEQ ID NO: 15) (GenBank Accession No: XP_001703126) |
| P37, respectively) | MQALATRPSAIRPTKAARRSSVVVRADGFIG (SEQ ID |
| | NO: 16) (GenBank Accession No: XP_001697230) |
| C. reinhardtii- | MAFALASRKALQVTCKATGKKTAAKAAAPKSSGVEFY |
| chlorophyll a/b | GPNRAKWLGPYSEN (SEQ ID NO: 17) (GenBank Accession |
| protein (cabII-1) | No: XP_001695353) |
| C. reinhardtii- | MAAVIAKSSVSAAVARPARSSVRPMAALKPAVKAAPVA |
| Rubisco small | APAQANQMMVWT (SEQ ID NO: 18) (GenBank Accession |
| subunit | No: XP_001702409) |
| C. reinhardtii- | MAAMLASKQGAFMGRSSFAPAPKGVASRGSLQVVAGL |
| ATPase-γ | KEY (SEQ ID NO: 19) (GenBank Accession No: |
| | XP_001696335) |
| Rubisco small | MASSMLSSATMVASPAQATMVAPFNGLKSSAAFPATRK |
| subunit | ANNDITSITSNGGRVNC (SEQ ID NO: 20) (GenBank |
| Arabidopsis | Accession No: GI:227206240) |
| Biotin carboxyl | MASSSFSVTSPAAAASVYAVTQTSSHFPIQNRSRRVSFRL |
| carrier protein | SAKPKLRFLSKPSRSSYPVVKA (SEQ ID NO: 21) |
| Arabidopsis | (GenBank Accession No: GI:1588550) |

Mitochondrial targeting peptides facilitate the targeting and translocation of cytosolically synthesized polypeptides into mitochondria. A mitochondrial targeting peptide useful with this invention can be any known or later identified plastid transit peptide sequence. Exemplary mitochondrial targeting peptides include those provided in Table 2, below.

TABLE 2
Amino acid sequences of representative mitochondrial targeting peptides.

| Source | Sequence |
|---|---|
| Superoxide Dismutase | MALRTLASKKVLSFPFGGAGRPLAAAASA&RG\hat{ }VTT (SEQ |
| (Maize | ID NO: 22) (NCBI Accession No: AQK85007.1) |
| Superoxide Dismutase | MALRTLASRKTLAAAALPLAAAAAARGVTT |
| (Rice) | (SEQ ID NO: 23) (NCBI Accession No: XP_015640127.1) |
| Superoxide Dismutase | MALRSLVTRKNLPSAFKAATGLGQLRGLQT |
| (Hevea brasiliensis) | (SEQ ID NO: 24) (NCBI Accession No: XP_021684793.1) |
| Superoxide Dismutase | MALRTLVSRRTLATGLGFRQQLRGLQT |
| (Nicotiana attenuata) | (SEQ ID NO: 25) (NCBI Accession No: XP_019256915.1) |
| Rieske-FeS protein | MLRVAGRRLSSSAARSSSTFFTRSSFTVTDDSSPARSPSP |
| (Potato) | SLTSSFLDQIRGFSSN |
| | (SEQ ID NO: 26) (NCBI Accession No: P37841.1) |
| Rieske-FeS protein | MLRVAGRRLSSSLSWRPAAAVARGPLAGAGVPDRDDD |
| (Maize) | SARGRSQPRFSIDSPFFVASRGFSSTETVVPRM |
| | (SEQ ID NO: 27) (NCBI Accession No: NP_001105561.1) |

Similarly, a plastid localization sequence facilitates the targeting and translocation of nuclear transcribed nucleic acids/polynucleotides into plastids. A plastid localization sequence useful with this invention can be any known or later identified plastid localization sequence. Exemplary plastid localization sequences include, but are not limited to, an Eggplant Latent Viroid (ELV) non-coding RNA sequence, an Avsunviroidae family non-coding RNA sequence, an Avocado sunblotch viroid (ASBVd) non-coding RNA sequence, a Peach latent mosaic viroid (PLMVd) non-coding RNA sequence, a Chrysanthemum chlorotic mottle viroid (CChMVd) non-coding RNA sequence, a (eIF4E) eukaryotic initiation factor 4E, and/or any combination thereof (see, e.g., Molina-Serrano et al. J. Virol. 81(8):4363-4366 (2007); Flores et al. Ann. Rev. Microbiol. 68:395-414 (2014)).

An exemplary ELV) non-coding RNA sequence useful with this invention includes

(SEQ ID NO: 28)
5′TTGGCGAAACCCCATTTCGACCTTTCGGTCTCATCAGGGGTGGCACAC
ACCACCCTATGGGGAGAGGTCGTCCTCTATCTCTCCTGGAAGGCCGGAGC
AATCCAAAAGAGGTACACCCACCCATGGGTCGGGACTTTAAATTCGGAGG
ATTCGTCCTTTAAACGTTCCTCCAAGAGTCCCTTCCCCAAACCCTTACTT
TGTAAGTGTGGTTCGGCGAATGTACCGTTTCGTCCTTTCGGACTCATCAG
GGAAAGTACACACTTTCCGACGGTGGGTTCGTCGACACCTCTCCCCCTCC
CAGGTACTATCCCCTTTCCAGGATTTGTTCCC3′

Further exemplary plastid localization sequences include, but are not limited to, those from:

Columnea latent viroid mRNA (GenBank Accession No.
M93686.1; locus CLEAAA):
(SEQ ID NO: 29)
5′CGGAACTAAACTCGTGGTTCCTGTGGTTCACACCTGACCCTGCAGCCA
TGCAAAAGAAAAAAGAACGGGAGGGAGAGCGCAAGAGCGGTCTCAGGAGC
CCCGGGGCAACTCAGACCGAGCGGGGTCTCGTGGTCGAGGGCGTACGCTG
TTCAGACAGGAGTAATCCCAGCTGAAACAGGGTTTTCACCCTTCCTTTCT
TCTGGTTTCCTTCCTCTGCTTCAGCGGCCTCGCCCGGAACCTCTTGACCA
GCGCAGGTGCTGACGCGACCGGTGGCATCACCGAGTTTGCTCAAGCCTCA
ACCTCCTTTTTCTCTATTCTGTAGCTTGGTCTCCGGGCGAGGGTGTTTAG
CCCTTGGAACCGCAGTTGGTTCCT3′;
or
Peach latent mosaic viroid (GenBank Accession No.
HQ342885.1):
(SEQ ID NO: 30)
5′CTCATAAGTTTCGCCGTATCTCAACGGCTCATCAGTGGGCTAAGCCCA
GACTTATGAGAGATTAGTCACCTCTCAGCCCCTCCACCTTGGGGTGCCCT
ATTCGAGGCACTGCAGTCTCGATAGAAAGGCTAAGCACGTCGCAATGACG
TAAGGTGGGACTTTTCCTTCTGGAACCAAGCGGTTGGTTCCGAGGGGGGT
GTGATCCAGGTACCGCCGTAGAAACTGGATTACGACGTCTACCCGGGATT
CCAACCCGGTCCCCTCCAGAAGTGATTCTGGATGAAGAGTCGTGCTTAGC
ACACTGATGAGTCTCTGAAATGAGACGAAACTCTTTTGA3′

Thus, in some embodiments, a nucleic acid of the invention may be linked to plastid localization sequence to target and translocate said nucleic acid into a plastid. Generally, a nucleic acid that is to be translocated into a plastid is linked at its 5′ end to the plastid localization sequence.

The present invention further provides plant cells and plants and parts therefrom produced by the methods described herein as well as progeny produced from said plants and plant cells. In some aspects, the methods of the invention further comprise regenerating a stably transformed plant or plant part from a stably transformed plant cell having a modified plastid genome. Means for regeneration can vary from plant species to plant species, but generally a suspension of transformed protoplasts or a petri plate containing transformed explants is first provided. Callus tissue is formed and shoots may be induced from callus and subsequently root. Alternatively, somatic embryo formation can be induced in the callus tissue. These somatic embryos germinate as natural embryos to form plants. The culture media will generally contain various amino acids and plant hormones, such as auxin and cytokinins. It may also be advantageous to add glutamic acid and proline to the medium, especially for such species as corn and alfalfa. Efficient regeneration will depend on the medium, on the genotype, and on the history of the culture. If these three variables are controlled, then regeneration is usually reproducible and repeatable.

The regenerated plants are transferred to standard soil conditions and cultivated in a conventional manner. The plants are grown and harvested using conventional procedures.

The particular conditions for transformation, selection and regeneration of a plant can be optimized by those of skill in the art. Factors that affect the efficiency of transformation include the species of plant, the target tissue or cell, composition of the culture media, selectable marker genes, kinds of vectors, and light/dark conditions. Therefore, these and other factors may be varied to determine an optimal transformation protocol for any particular plant species. It is recognized that not every species will react in the same manner to the transformation conditions and may require a slightly different modification of the protocols disclosed herein. However, by altering each of the variables, an optimum protocol can be derived for any plant species.

Further, the genetic properties engineered into the transgenic seeds and plants, plant parts, and/or plant cells of the present invention described herein can be passed on by sexual reproduction or vegetative growth and therefore can be maintained and propagated in progeny plants. Generally, maintenance and propagation make use of known agricultural methods developed to fit specific purposes such as harvesting, sowing or tilling.

Thus, additionally provided herein are seeds produced from said plants and crops that comprise a plurality of the plants of the invention planted together in an agricultural field, a golf course, a residential lawn, a road side, an athletic field, and/or a recreational field.

The present invention further provides a product or products produced from the stably transformed plant, plant cell or plant part of the invention. In particular embodiments, the present invention further provides a product produced from the seed of the stably transformed plant.

In some aspects of the invention, a product can be a product harvested from the transgenic plants, plant parts, plant cells, and/or progeny thereof, or crops of the invention, as well as a processed product produced from said harvested product. A harvested product can be a whole plant or any plant part, as described herein, wherein said harvested product comprises a heterologous polynucleotide of the invention. Non-limiting examples of a harvested product include a seed, a fruit, a flower or part thereof (e.g., an anther, a stigma, and the like), a leaf, a stem, and the like. In some embodiments, a processed product includes, but is not limited to, a flour, meal, oil, starch, cereal, and the like produced from a harvested seed of the invention. In some embodiments, the product produced from the stably transformed plants, plant parts and/or plant cells can include, but is not limited to, biofuel, food, drink, animal feed, fiber, commodity chemicals, cosmetics, and/or pharmaceuticals

The invention will now be described with reference to the following examples. It should be appreciated that these examples are not intended to limit the scope of the claims to the invention, but are rather intended to be exemplary of certain embodiments. Any variations in the exemplified methods that occur to the skilled artisan are intended to fall within the scope of the invention.

### EXAMPLES

### Example 1

### CRISPR Editing of a Plastid Genome

Nuclear (or extrachromosomal) transformation of a plant to express Cas9 polypeptide that is targeted to the chloroplast and a guide RNA that is also targeted to the chloroplast results in targeting the Cas9 polypeptide to the desired chloroplast DNA (cpDNA) sequence, thereby allowing either homologous recombination for efficient insertion of transgenes into the cpDNA or non-homologous end joining (NHEJ) mutations in the cpDNA.

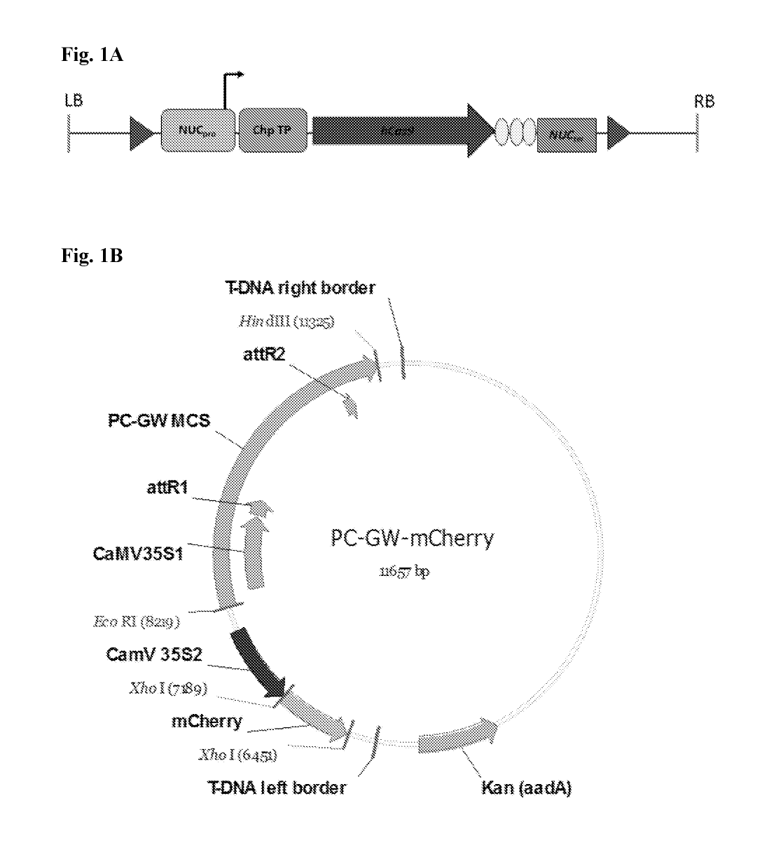

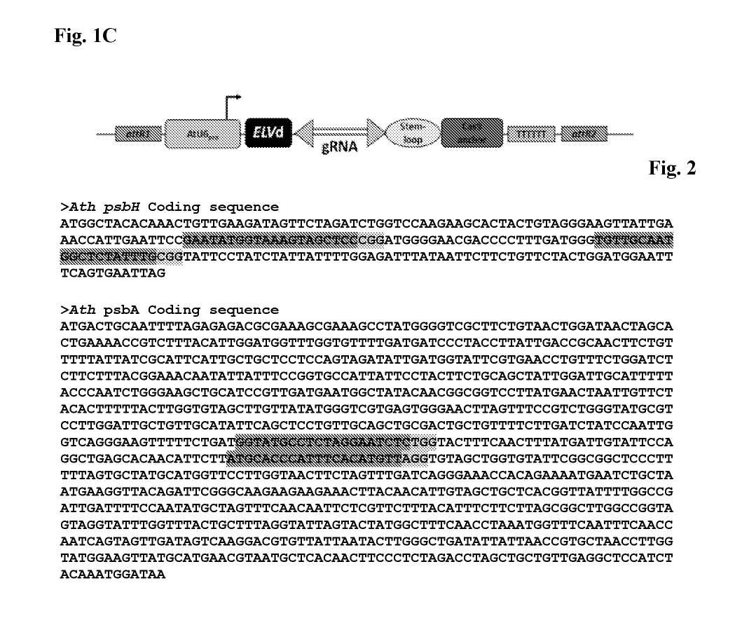

An exemplary transformation construct of the invention comprises a Cas9 polypeptide fused to a chloroplast targeting sequence of, for example, ribulose-1,5-bisphosphate carboxylase/oxygenase small subunit (rbcS) (see, FIG. 1A) that is sub-cloned into the attR1 and attR2 sites of a binary vector (e.g., PC-GW-Bar vector (Genbank: KP826773)) (see, FIG. 1B) by gateway assisted recombination and transformed into the nucleus of Arabidopsis by Agrobacterium-mediated plant transformation method. The CaMV 35S constitutive promoter may be used for driving the transcription of the Cas9 gene.

Homozygous transgenic lines expressing the Cas9 protein in the chloroplast are identified (genotyping, Western Blot). Different functional isoforms of Cas9 will be tested for efficiency and lethality. Stable transgenic lines expressing the Cas9 polypeptide will be transformed by a Agrobacterium-mediated plant transformation method with a construct (see, FIG. 1C) comprising a guide RNA (gRNA) to target the psbA or psbH to generate photosynthesis mutants that can be readily screened. Exemplary gRNA target sequences that may be used to create indels inpsbH or psbA gene are shown in FIG. 2. The Eggplant Latent Viroid (ELVd) derived non-coding RNA sequence may be used for importing the gRNA from the nucleus into the chloroplast as reported in (Gomez et al., 2012). T1 seedlings will be genotyped for mutations in the target genes.

Other forms of Cas9, e.g. deactivated Cas9 (dCas9), fdCas9 or nicking Cas9, may be used as described herein for gene activation/inactivation applications. In some embodiments, the plastids may also be transformed with RNase III, which may assist in activating the CRISPR pathway (Deltcheva et al., 2011).

### Example 2

### A method for Producing cDNA in a Plastid Using Reverse Transcriptase

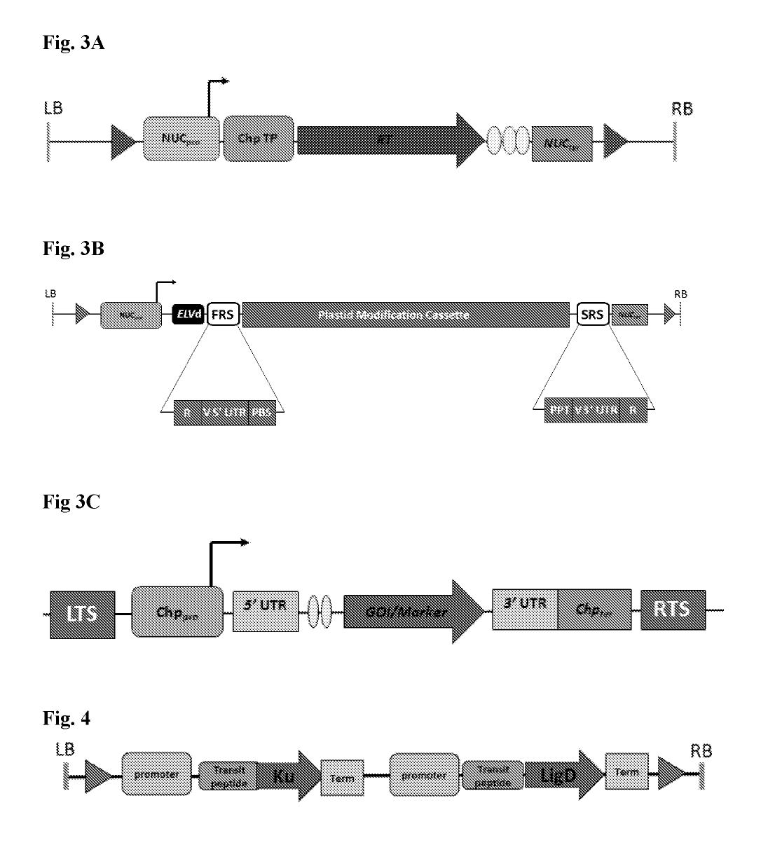

Arabidopsis plants are transformed (nuclear (or extrachromosomal)) with (a) a nucleotide sequence encoding a reverse transcriptase polypeptide that is targeted to the chloroplast (see, e.g., FIG. 3A) and a (b) a recombinant nucleic acid comprising (i) a plastid modification cassette, (ii) a first recognition site located immediately 5′ of the plastid modification cassette, (iii) a second recognition site located immediately 3′ of the plastid modification cassette, and (iv) a plastid localization sequence operably located 5′ of the first recognition site (see, e.g., FIG. 3B). Exemplary reverse transcriptase polypeptides can be obtained from Ty4 (copia), M-MLV, and/or HIV-1. A plastid localization sequence useful with the invention can be, for example, an Eggplant Latent Viroid derived non-coding RNA sequence (ELVd) which can target the guide nucleic acid to the chloroplast. The first recognition site can be a tRNA-derived recognition sequence (PBS) and the second recognition site can be a polypurine tract (PPT) both of which are involved in the function of the reverse transcriptase (see, e.g., FIG. 3B). The plastid modification cassette comprises a first homology arm and a second homology arm, and an intervening synthetic nucleotide sequence located between the first and second homology arms and comprising a polynucleotide sequence of interest (POI) (see, e.g., FIG. 3C). The homozygous stable transgenic Arabidopsis lines that are generated and express the chloroplast-targeted reverse transcriptase are evaluated for their ability to survive and produce intact and active reverse transcriptase protein in chloroplasts. Reverse transcriptase activity is assayed by an in vitro activity assay (Sigma and Life Technologies Assay Kits) and RNAseH activity according to Kaufmann et al. (2009). In some constructs, a generic promoter and terminator functional in a plant chloroplast can be included, for example, that from ribulose-1,5-bisphosphate carboxylase/oxygenase large subunit.

Additionally, a transient tobacco leaf transformation systems may be used to test the activity of reverse transcriptase in chloroplasts.

The combination of expression of a reverse transcriptase and a plastid modification cassette as described herein in the plastid will result in the generation of a double stranded DNA of the desired transgene (POI) in the chloroplast for integration into the desired cpDNA (chloroplast DNA) locus by homologous recombination.

### Example 3

### Efficient Transformation and/or Editing of Plastid DNA and Production of Homoplasmic Transplastomic Plants

A plant cell is transformed (nuclear (or extrachromosomal)) with (a) a polynucleotide encoding a Cas9 polypeptide fused to a plastid transit peptide and operably linked to a promoter; (b) a guide nucleic acid operably linked at the 5′ end to a chloroplast localization sequence; (c) a polynucleotide encoding a reverse transcriptase polypeptide fused to a plastid transit peptide; and (d) a recombinant nucleic acid comprising (i) a plastid modification cassette, (ii) a first recognition site located immediately 5′ of the plastid modification cassette, (iii) a second recognition site located immediately 3′ of the plastid modification cassette, and (iv) a plastid localization sequence operably located 5′ of the first recognition site, thereby modifying and/or transforming the chloroplast genome of said plant cell. Said plant cell can be regenerated into a homoplasmic transplastomic plant having all chloroplasts with modified genomes that are identical and not segregating.

### Example 4

### Non-Homologous End-joining DNA Repair in Plastids

While plastids have an effective mechanism to repair double-stranded DNA breaks using homologous recombination (HR or Homology-Derived Repair HDR), a repair mechanism using Non-Homologous End-Joining (NHEJ) has not been shown to exist in chloroplasts. This is likely due to the prokaryotic origin of the chloroplasts. Under NHEJ, the two DNA ends are joined in an error-prone manner, resulting in random insertions and deletions (indels). Plants comprising the polynucleotides and nucleic acids described in Examples 1 to 3, will be further transformed with a polynucleotide encoding an ATP-dependent DNA ligase D (LigD) fused to a plastid transit peptide and a polynucleotide encoding a DNA-binding protein Ku (Ku) fused to a plastid transit peptide to enable NHEJ and HDR, thereby improving the efficiency of Cas9-based genome editing further.

Thus, for example, a nucleotide sequence encoding a Mycobacterium tuberculosis (Mt) Ku-like protein fused to a plastid transit peptide (e.g., rbcS) and a nucleotide sequence encoding an ATP-dependent DNA ligase (LigD) fused to a plastid transit peptide (e.g., rbcS), can be introduced into a plant to perform non-homologous end joining (NHEJ) in the chloroplast (see, e.g., FIG. 4).

Thus, heterologous expression of Ku and LigD polypeptides provides Cas9-directed genome editing via NHEJ repair in plastids. This may assist in the generation of site-specific mutations (indels) to evaluate gene function in plastids or modify specific functions. Expression of Ku and LigD enzymes in plastids may increase the efficiency of Cas9-based plastid DNA transformation as well. The polynucleotides encoding Ku and LigD polypeptides can be stably or transiently transformed into the plant nucleus as fusion constructs with a chloroplast target sequence (see, e.g., FIG. 4).

The above examples clearly illustrate the advantages of the invention. Although the present invention has been described with reference to specific details of certain embodiments thereof, it is not intended that such details should be regarded as limitations upon the scope of the invention except as and to the extent that they are included in the accompanying claims.

### Example 5

### Expression of Reverse Transcriptase in a Stably Transformed Plant

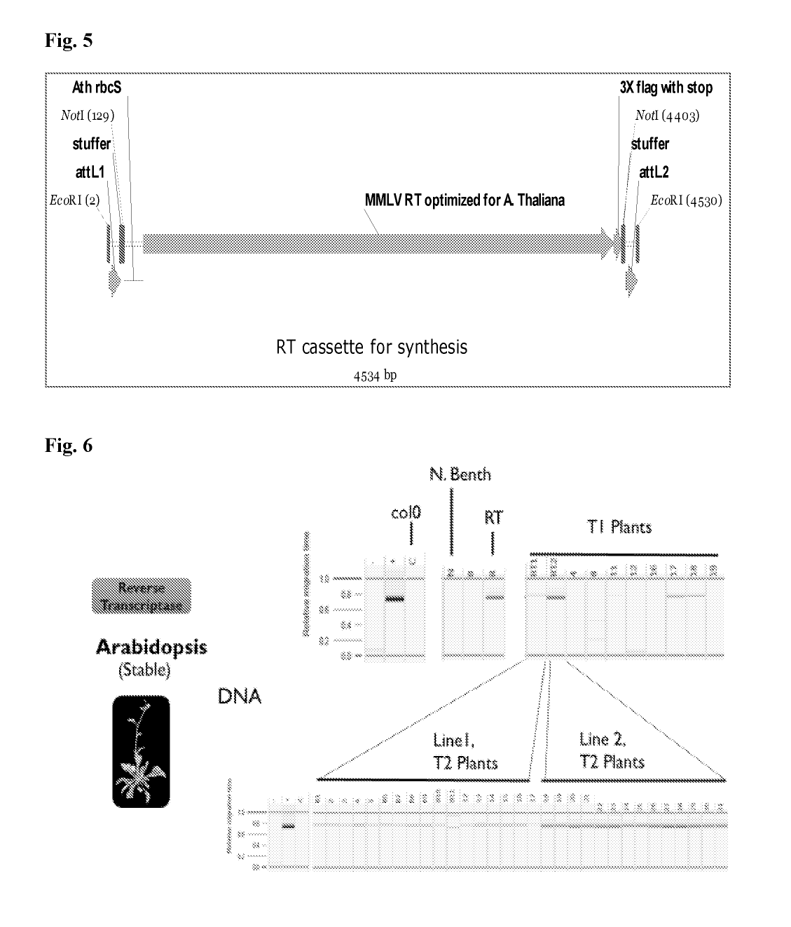

We have expressed a chloroplast localized reverse transcriptase in plants. An exemplary construct is shown in FIG. 5. Here, the gene sequence is codon optimized for nuclear expression in Arabidopsis thaliana. The RT cassette was synthesized and cloned at the EcoRI site vector pUC57 vector by Genscript. The cassette was further sub-cloned into PC-GW-Bar (plant transformation vector) (Genbank accession: KP826773) by gateway cloning (Sequence of protein and gene cassette attached in separate document). Maintenance of the reverse transcriptase (RT) gene and expression of the protein is heritable. FIG. 6 shows that expression of this protein by plants corresponds with the genuine activity of reverse transcription. Not only does FIG. 6 show these plants carry the gene, but also that it is passed on to subsequent generations.

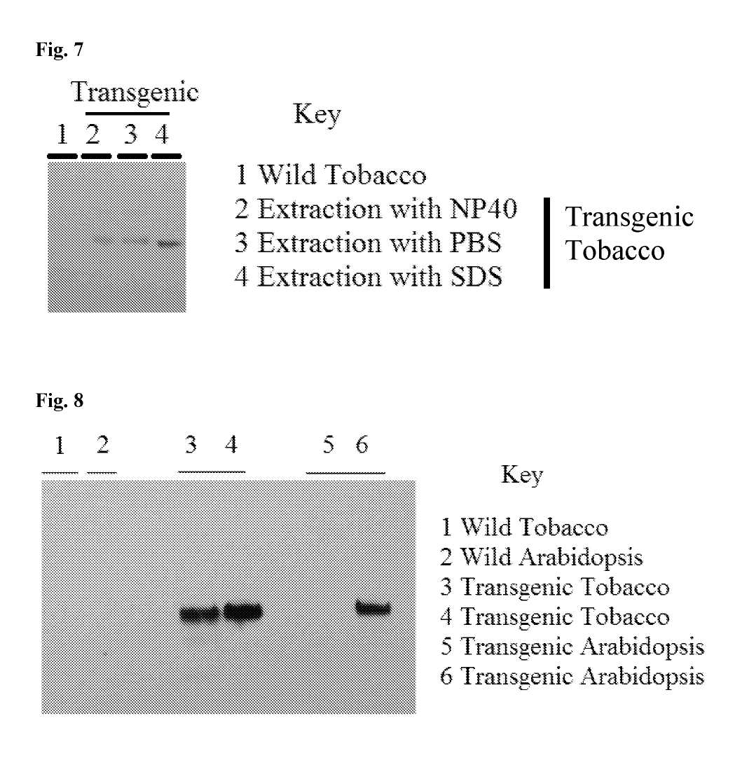

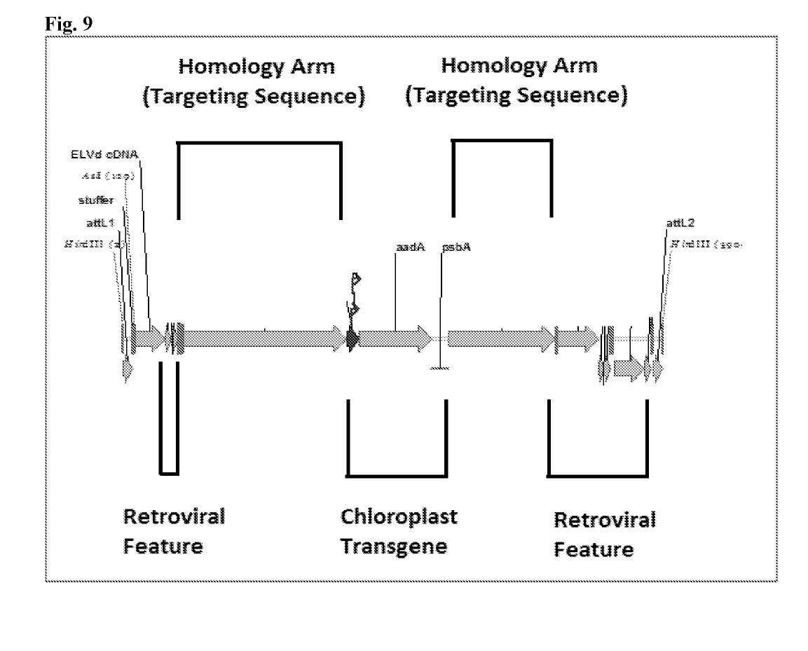

FIGS. 7 and 8 provide western blots of protein extracted from tobacco and Arabidopsis. The presence of a band indicates the presence of reverse transcriptase protein. FIG. 7 provides a comparison between extraction techniques in recovering reverse transcriptase produced by tobacco. Lane 1 is protein extracted from normal tobacco, whereas lanes 2, 3, and 4 are protein extracted from transgenic tobacco expressing chloroplast targeted reverse transcriptase. Lanes 2, 3, and 4 differ in extraction methods, the major difference being that protein in lanes 2 and 4 are extracted with high levels of detergent, whereas lane 3 is essentially just water and salt. Because we can identify protein in lane 3, we can safely assume that chloroplast localized reverse transcriptase is soluble. FIG. 8 shows that RT protein can be produced in stable lines of Arabidopsis thaliana.

As a preliminary examination of reverse transcriptase activity in the transgenic plants, plant material was ground with a salt buffer and used in a fluorescent assay that detects the production of DNA from RNA (EnzCheck Reverse Transcriptase assay). In this experiment, the presence of fluorescent signal was used as a proxy for the production of DNA from RNA. The preliminary results shown in Table 3 show that the transgenic plants exhibit some degree of reverse transcriptase activity, while non-transgenic plants do not, thus showing that the system is working as intended.

TABLE 3
Reverse transcriptase activity

| Sample | Fluorescence Value |
|---|---|
| Commerical RT | 90 |
| Wild Arabidopsis | 0 |
| Wild Tobacco | 0 |
| Transgenic Arabidopsis | 9 |
| Transgenic Tobacco | 10 |

### Example 6

### Design of Plastid Transformation Expression Cassettes

Four different plastid modification cassettes have been designed and made. The major difference between each plastid modification cassette is the sequence of the homology arms. The homology arms determine where a chloroplast transgene will integrate in a chloroplast genome. Retroviral features are necessary for the action of reverse transcriptase as described in the original project design and our patent filing. Homology arms are required for the integration of the PTEC into chloroplast genomes. These regions must be complementary to the chloroplast of a plant for transformation to take place.

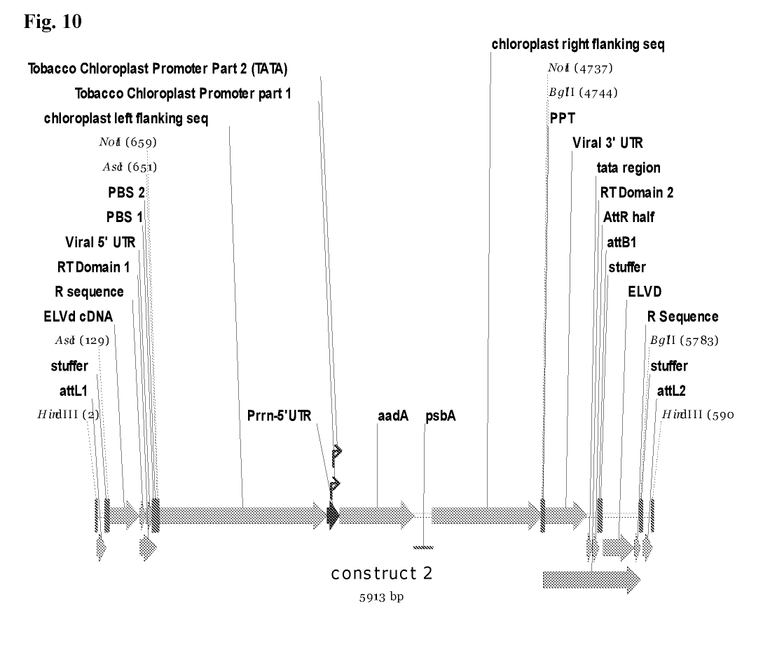

Four different constructs were made using different homology arms to assess if the location of transgene integration has an impact on the efficiency of the system. A generic plastid modification cassette is shown in FIG. 9. Each of the plastid modification cassette constructs described in this example carry the features of the exemplary cassette shown in FIG. 9 and are identical in sequence with one another other than the exception of the homology arms. FIG. 10 shows the exemplary construct of FIG. 9 in more detail. Example plastid modification cassette constructs include the nucleotide sequence of SEQ ID NO:31 (PTECv1), the nucleotide sequence of SEQ ID NO:32 (PTECv4), and the nucleotide sequence of SEQ ID NO:33. (PTECv5)

### Example 7

### Plastid Modification Cassette Expression in Plants

We have found that at least 3 different plastid modification cassette RNAs can be carried and produced by plants. The genes coding the plastid modification cassette and expression of the plastid modification cassette RNA can be carried to at least the second generation. However, one of the plastid modification cassette constructs appears to affect the viability of the derived seed from the plants carrying that construct.

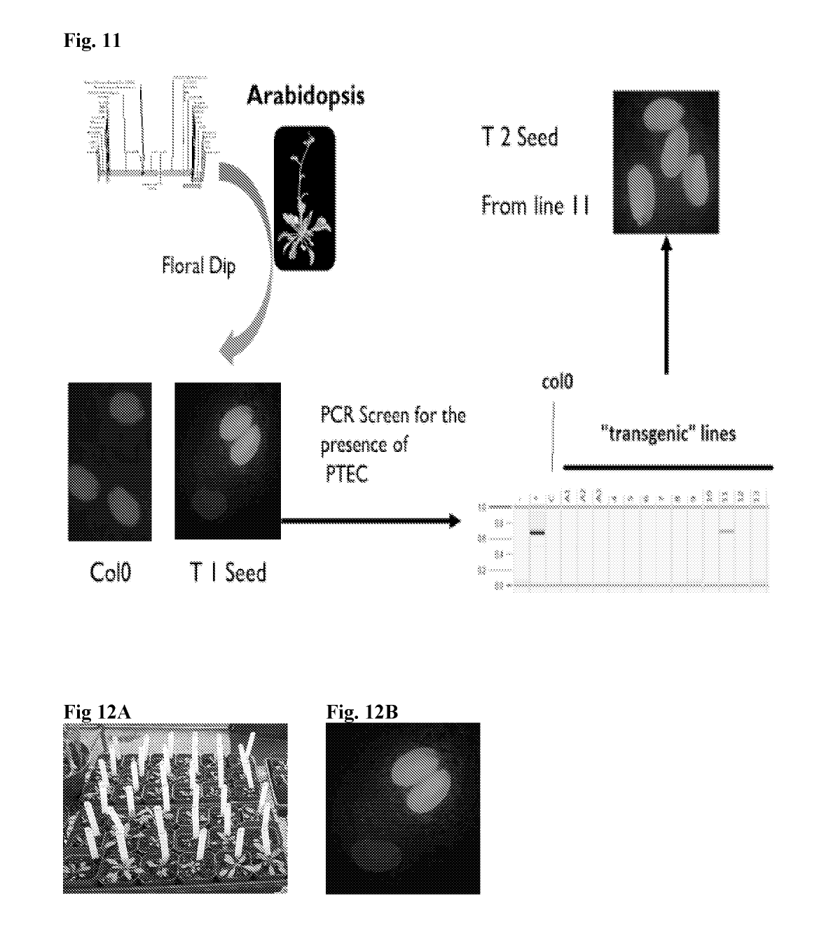

FIG. 11 shows an exemplary workflow design for generating transgenic Arabidopsis and confirming the presence of the transgene introduced in a plastid modification cassette. The plastid modification cassette is inserted into Arabidopsis cells via agrobacterium using the floral dip method. Here, the plastid modification cassette construct co-expresses a fluorescent protein (marker gene) that allows us to identify plants containing the construct by segregating seed. Plants were verified to be transgenic by PCR analysis.

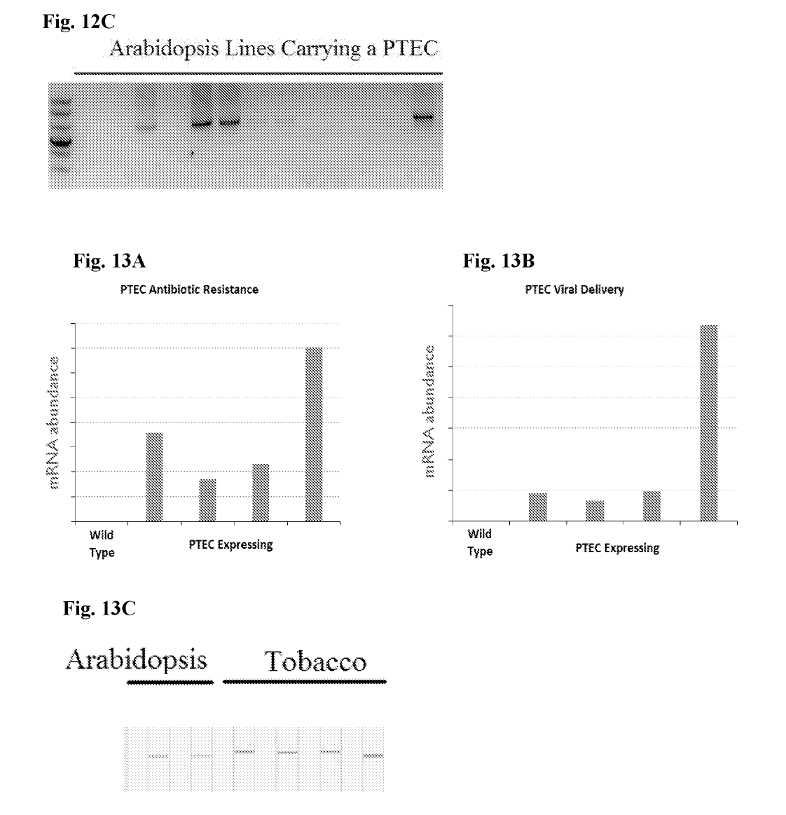

FIGS. 12A-12C show that the transgenes in the plastid modification cassette are heritable and expressed. Seeds carrying the plastid modification cassette were identified using mCherry expression and fluorescence sorting (FIG. 12A). About 80 plants have been identified using mCherry fluorescence sorting are growing in a greenhouse (FIG. 12B). FIG. 12C shows Arabidopsis thaliana lines, which carry the full length plastid modification cassette. The plants which carry the cassette are revealed by the presence of a single dark band. These plants produced progeny (seed), which we have collected are currently screening to produce a second generation.

FIGS. 13A-13C show that plastid modification cassette RNA is expressed and the expression is heritable in both tobacco and Arabidopsis. Quantitative PCR assays are used to show the abundance of RNA in transformed and wild type tobacco expressing the plastid modification cassette (FIG. 13A and FIG. 13B). The two assays use the same leaf material but target different locations on the plastid modification cassette to probe. Using PCR, material from plastid modification cassette carrying lines of Arabidopsis was compared with tobacco in order to demonstrate that plastid modification cassette expression was maintained in a stable, heritable system (FIG. 13C).

### Example 9

### Plastid Targeted Cas9

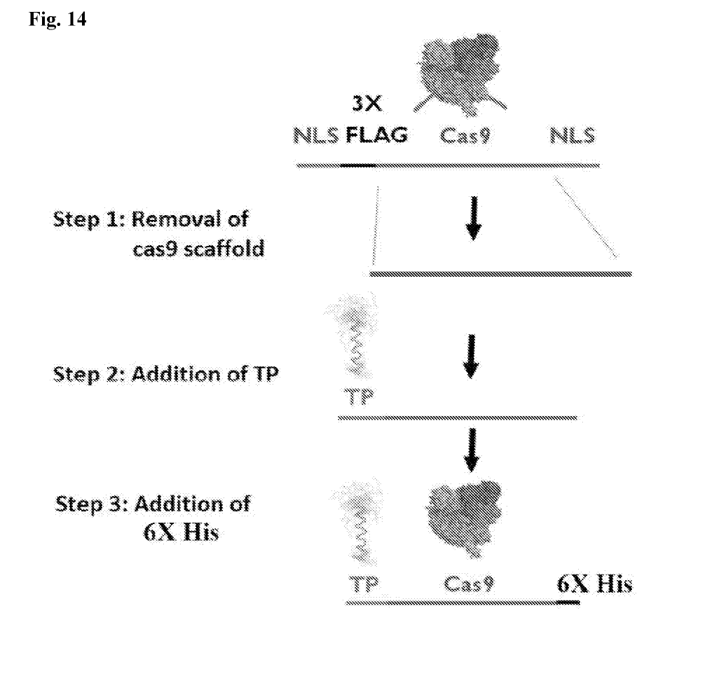

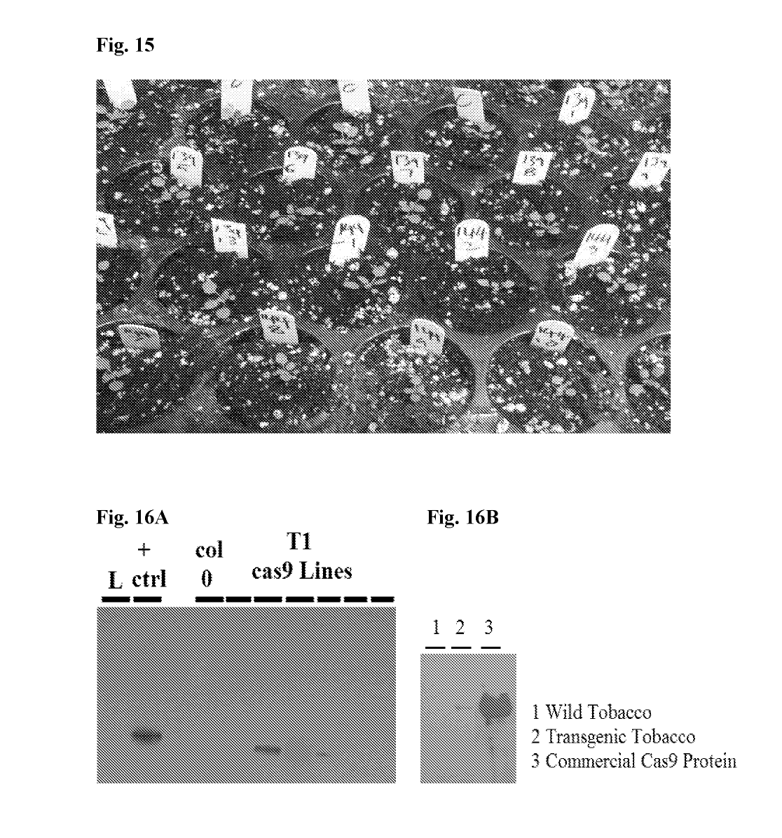

Cas9 can be modified to be targeted to the chloroplast as shown in FIG. 14, which is an exemplary process that replaces the nuclear localization signal (NLS) of the Cas9 with a chloroplast transit peptide (TP) sequence. First and second generation Arabidopsis plants have been generated (FIG. 15). These plants exhibit no obvious differences compared to wild type plants. FIGS. 16A-16B show that chloroplast targeted Cas9 is produced by the transgenic Arabidopsis (FIG. 16A) and tobacco (FIG. 16B) plants.

### Example 10

### CRISPR Cas Guide Designs for Chloroplast Editing

We generated two separate sgRNA expression constructs to test the interaction between the sgRNA, which is necessary for the action of Cas9, and the ELVD, which enabled chloroplast entry. In these two constructs, the order of the ELVD and sgRNA are reversed with the ELVD located at the 5′ end of the sgRNA in one construct and the ELVD located at the 3′ end of the sgRNA in the other construct. The sgRNA cassettes were synthesized by Integrated DNA technologies and cloned in PC-GW-EGFP (plant transformation vector) by gateway cloning.

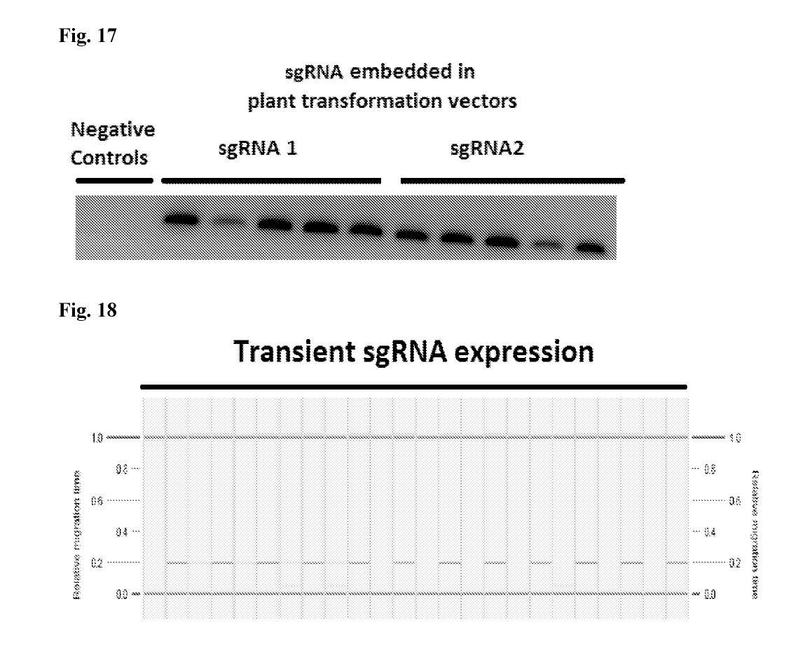

Ten sgRNA cassettes embedded in plant transformation vectors were identified using PCR using primers specific to each unique 20 nt target site (FIG. 17). The amplified band shown in the figure demonstrated the presence of the correct casette sequence. Additional sequencing data additioanlly confirms the proper construction of each casette.

### Example 11

### Plants Expressing sgRNA Containing Chloroplast Targeting Untranslated Regions (UTRs)

Tobacco carrying the sgRNA transgenes as described above was shown to produce a full length sgRNA, including the chloroplast targeting UTR (FIG. 18).

### Example 12

### Constructs Comprising sgRNA with Nos Promoter

Additional constructs were prepared using the Nos promoter instead of the U6 promoter. These will be used to generate transgenic plants.

### Example 13

### Plants transformed and Expressing Reverse Transcriptase and the Plastid Modification Cassette Transgene

Plants transformed with and expressing reverse transcriptase as described above were transformed with two vectors coding for plastid modification cassettes. This combination yields seed that is mCherry fluorescent and resistant to the antibiotic/herbicide glufosinate. Seed was screened for mCherry fluorescence and plated on agar containing glufosinate. Plantlets appeared 14 days after planting in soil and were transfer to greenhouse.

### REFERENCES

Bock, Ralph. “Engineering Plastid Genomes: Methods, Tools, and Applications in Basic Research and Biotechnology.” Annual review of plant biology 66 (2015): 211-241.
Bock, Ralph. “Plastid biotechnology: prospects for herbicide and insect resistance,metabolic engineering and molecular farming.” Current Opinion in Biotechnology 18, no. 2 (2007): 100-106.
Borner, T., Zhelyazkova, P., Legen, J., and C. Schmitz-Linneweber “Chloroplast gene expression—RNA synthesis and processing” in S. M. Theg, F. A. Wollman (Eds.), Advances in Plant Biology, Vol. 5: Plastid Biology, Springer, Dordrecht (2014), pp. 3-47
Boynton, John E., Nicholas W. Gillham, Elizabeth H. Harris, Jonathan P. Hosler, Anita M. Johnson, Allan R. Jones, Barbara L. Randolph-Anderson, Dominique Robertson, Ted M. Klein, and Katherine B. Shark. “Chloroplast transformation in Chlamydomonas with high velocity microprojectiles.” Science 240, no. 4858 (1988): 1534-1538.
Corriveau, Joseph L., and Annette W. Coleman. “Rapid screening method to detect potential biparental inheritance of plastid DNA and results for over 200 angiosperm species.” American Journal of Botany (1988): 1443-1458.
Daniell, Henry, Gricel Ruiz, Bela Denes, Laurence Sandberg, and William Langridge. “Optimization of codon composition and regulatory elements for expression of human insulin like growth factor-1 in transgenic chloroplasts and evaluation of structural identity and function.” BMC biotechnology 9, no. 1 (2009): 33.
Day, A. and Goldschmidt-Clermont, M. (2011), The chloroplast transformation toolbox: selectable markers and marker removal. Plant Biotechnology Journal, 9: 540-553. doi: 0.1111/j.1467-7652.2011.00604.x
Golds, Timothy, Pal Maliga, and Hans-Ulrich Koop. “Stable plastid transformation in PEG-treated protoplasts of Nicotiana tabacum.” Nature Biotechnology 11, no. 1 (1993): 95-97.
Gomez, Gustavo, and Vicente Pallas. “Studies on subcellular compartmentalization of plant pathogenic noncoding RNAs give new insights into the intracellular RNA-traffic mechanisms.” Plant physiology 159, no. 2 (2012): 558-564.
Maliga, Pal. “Plastid transformation in flowering plants.” In Genomics of chloroplasts and mitochondria, pp. 393-414. Springer Netherlands, 2012.
Meyers, B., Zaltsman, A., Lacroix, B., Kozlovsky, S. V., & Krichevsky, A. (2010). Nuclear and plastid genetic engineering of plants: comparison of opportunities and challenges. Biotechnology advances, 28(6), 747-756.
Oey, Melanie, Marc Lohse, Bernd Kreikemeyer, and Ralph Bock. “Exhaustion of the chloroplast protein synthesis capacity by massive expression of a highly stable protein antibiotic.” The plant journal 57, no. 3 (2009): 436-445.
Quesada-Vargas, Tania, Oscar N. Ruiz, and Henry Daniell. “Characterization of heterologous multigene operons in transgenic chloroplasts. Transcription, processing, and translation.” Plant physiology 138, no. 3 (2005): 1746-1762.
Ruf, Stephanie, Daniel Karcher, and Ralph Bock. “Deteimining the transgene containment level provided by chloroplast transformation.” Proceedings of the National Academy of Sciences 104, no. 17 (2007): 6998-7002.
Ruf, Stephanie, Marita Hermann, Irving J. Berger, Helaine Carrer, and Ralph Bock. “Stable genetic transformation of tomato plastids and expression of a foreign protein in fruit.” Nature biotechnology 19, no. 9 (2001): 870-875.
Ruhlman, Tracey, Dheeraj Verma, Nalapalli Samson, and Henry Daniell. “The role of heterologous chloroplast sequence elements in transgene integration and expression.” Plant physiology 152, no. 4 (2010): 2088-2104.
Sack, Markus, Anna Hofbauer, Rainer Fischer, and Eva Stoger. “The increasing value of plant-made proteins.” Current opinion in biotechnology 32 (2015): 163-170.
Staub, Jeffrey M., and Pal Maliga. “Expression of a chimeric uidA gene indicates that polycistronic mRNAs are efficiently translated in tobacco plastids.” The Plant Journal 7, no. 5 (1995): 845-848.
Stoger, Eva, Rainer Fischer, Maurice Moloney, and Julian K-C. Ma. “Plant molecular pharming for the treatment of chronic and infectious diseases”. Annual review of plant biology 65 (2014): 743-768.
Svab, Zora, and Pal Maliga. “Exceptional transmission of plastids and mitochondria from the transplastomic pollen parent and its impact on transgene containment.” Proceedings of the National Academy of Sciences 104, no. 17 (2007): 7003-7008.
Svab, Zora, Peter Hajdukiewicz, and Pal Maliga. “Stable transformation of plastids in higher plants.” Proceedings of the National Academy of Sciences 87, no. 21 (1990): 8526-8530.
Zhang, J., Khan, S. A., Hasse, C., Ruf, S., Heckel, D. G., & Bock, R. (2015). Full crop protection from an insect pest by expression of long double-stranded RNAs in plastids. Science, 347(6225), 991-994.

That which is claimed is:

1. A method of modifying a plastid genome of a plant cell, comprising
introducing into a plant cell:
(a) a polynucleotide encoding a reverse transcriptase (RT) polypeptide fused to a plastid transit peptide;
(b) a recombinant nucleic acid comprising (i) a plastid modification cassette, (ii) a first recognition site located immediately 5′ of the plastid modification cassette, (iii) a second recognition site located immediately 3′ of the plastid modification cassette, and (iv) a plastid localization sequence operably located 5′ of the first recognition site; and
(c) a polynucleotide encoding a sequence-specific nuclease fused to a plastid transit peptide, thereby modifying the plastid genome of said plant cell.

2. A method of modifying a plastid genome of a plant cell, comprising
introducing into a plant cell a polynucleotide encoding an ATP-dependent DNA ligase D (LigD) fused to a plastid transit peptide and a polynucleotide encoding a DNA-binding protein Ku (Ku) fused to a plastid transit peptide, thereby modifying the plastid genome of said plant cell.

3. The method of claim 2, further comprising introducing into the plant cell a polynucleotide encoding a sequence-specific nuclease fused to a plastid transit peptide.

4. A method of modifying a plastid genome of a plant cell, comprising:
introducing into a plant cell
(a) a recombinant nucleic acid linked to a plastid localization sequence and comprising a polynucleotide encoding a reverse transcriptase polypeptide and a sequence-specific nuclease; and
(b) a recombinant nucleic acid comprising (i) a plastid modification cassette, (ii) a first recognition site located immediately 5′ of the plastid modification cassette, (iii) a second recognition site located immediately 3′ of the plastid modification cassette, and (iv) a plastid localizing sequence operably located 5′ of the first recognition site, thereby modifying the plastid genome of the plant cell.

5. The method of claim 3 or claim 4, wherein the sequence-specific nuclease is a Cas9 nuclease or a Cpf1 nuclease, the method further comprising introducing a guide nucleic acid linked to a plastid localization sequence.

6. The method of claim 3 or claim 4, wherein the sequence-specific nuclease is a Cpf1 nuclease, transcription activator-like (TAL) effector nuclease (TALEN), a zinc-finger nuclease (ZFN), and/or a meganuclease.

7. A method of producing a plant cell having a modified plastid genome, comprising
introducing into a plant cell:
(a) a polynucleotide encoding a reverse transcriptase (RT) polypeptide fused to a plastid transit peptide;
(b) a recombinant nucleic acid comprising (i) a plastid modification cassette, (ii) a first recognition site located immediately 5′ of the plastid modification cassette, (iii) a second recognition site located immediately 3′ of the plastid modification cassette, and (iv) a plastid localization sequence operably located 5′ of the first recognition site; and
(c) a polynucleotide encoding a sequence-specific nuclease fused to a plastid transit peptide, thereby producing a plant cell having a modified plastid genome.

8. A method of producing a plant cell having a modified plastid genome, comprising introducing into a plant cell a polynucleotide encoding an ATP-dependent DNA ligase D (LigD) fused to a plastid transit peptide and a polynucleotide encoding a DNA-binding protein Ku (Ku) fused to a plastid transit peptide, thereby modifying the plastid genome of said plant cell.

9. The method of claim 8, further comprising introducing into said plant cell a sequence-specific nuclease fused to a plastid transit peptide.

10. A method of producing a plant cell having a modified plastid genome, comprising:
introducing into a plant cell
(a) a recombinant nucleic acid linked to a plastid localization sequence and comprising a polynucleotide encoding a reverse transcriptase polypeptide and a sequence-specific nuclease; and
(b) a recombinant nucleic acid comprising (i) a plastid modification cassette, (ii) a first recognition site located immediately 5′ of the plastid modification cassette, (iii) a second recognition site located immediately 3′ of the plastid modification cassette, and (iv) a plastid localizing sequence operably located 5′ of the first recognition site, thereby modifying the plastid genome of the plant cell.

11. The method of claim 9 or claim 10, wherein the sequence-specific nuclease is a Cas9 nuclease or a Cpf1 nuclease, the method further comprising introducing a guide nucleic acid linked to a plastid localization sequence.

12. The method of claim 10, wherein the sequence-specific nuclease is a Cpf1 nuclease, transcription activator-like (TAL) effector nuclease (TALEN), a zinc-finger nuclease (ZFN), and/or a meganuclease.

13. A method of expressing a polynucleotide sequence of interest (POI) in a plastid, comprising introducing into a plant cell: (a) a polynucleotide encoding a reverse transcriptase polypeptide fused to a plastid transit peptide and a sequence-specific nuclease fused to a plastid transit peptide, or a polynucleotide linked to a plastid localization sequence and encoding a reverse transcriptase polypeptide and a sequence-specific nuclease; and (b) a recombinant nucleic acid comprising (i) a plastid modification cassette, (ii) a first recognition site located immediately 5′ of the plastid modification cassette, (iii) a second recognition site located immediately 3′ of the plastid modification cassette, and (iv) a plastid localization sequence operably located 5′ of the first recognition site, wherein said plastid modification cassette comprises a POI, thereby expressing the POI in a plastid.

14. A method of transforming a plastid genome, comprising: introducing into a plant cell: (a) a polynucleotide encoding a reverse transcriptase polypeptide fused to a plastid transit peptide and a sequence-specific nuclease fused to a plastid transit peptide, or a polynucleotide linked to a plastid localization sequence and encoding a reverse transcriptase polypeptide and a sequence-specific nuclease; and (b) a recombinant nucleic acid comprising (i) a plastid modification cassette, (ii) a first recognition site located immediately 5′ of the plastid modification cassette, (iii) a second recognition site located immediately 3′ of the plastid modification cassette, and (iv) a plastid localization sequence operably located 5′ of the first recognition site, wherein said plastid modification cassette comprises a POI, thereby transforming said plastid genome.

15. The method of any one of claim 1, 3, 7, 9, 13 or 14, wherein introducing a sequence-specific nuclease comprises introducing:
a polynucleotide encoding a Cpf1 nuclease fused to a plastid transit peptide or encoding a Cas9 nuclease fused to a plastid transit peptide; and
a guide nucleic acid linked to a plastid localization sequence, thereby modifying the plastid genome of said plant cell.

16. The method of any one of claim 1, 3, 7, 9, 13 or 14, wherein introducing a sequence-specific nuclease comprises introducing:
a polynucleotide encoding a transcription activator-like (TAL) effector nuclease (TALEN) fused to a plastid transit peptide, wherein the TALEN comprises a TAL effector DNA-binding domain fused to a DNA cleavage domain.

17. The method of any one of claim 1, 3, 7, 9, 13 or 14, wherein introducing a sequence-specific nuclease comprises introducing:
a polynucleotide encoding a zinc-finger nuclease (ZFN) fused to a plastid transit peptide, wherein the ZFN comprises a zinc finger DNA-binding domain fused to a DNA-cleavage domain.

18. The method of any one of claim 1, 3, 7, 9, 13 or 14, wherein introducing a sequence-specific nuclease comprises introducing:
a polynucleotide encoding a meganuclease fused to a plastid transit peptide.

19. The method of any one of claims 1 to 18, wherein the polynucleotide encoding a sequence-specific nuclease, the polynucleotide encoding a RT polypeptide, the polynucleotide encoding LigD, and/or the polynucleotide encoding Ku are operably linked to one or more promoters and optionally, operably linked to one or more terminators.

20. The method of any one of claim 1, 4, 5, 7, 10, 11, or 15, wherein the recombinant nucleic acid and/or the guide nucleic acid are each operably linked to a promoter and optionally, operably linked to a terminator.

21. The method of any one of claim 5, 11, 15 or 20, wherein the guide nucleic acid comprises a recombinant CRISPR array or a recombinant CRISPR array and a recombinant trans-activating CRISPR (tracr) nucleic acid.

22. The method of claim 21, wherein the recombinant CRISPR array and the recombinant tracr nucleic acid are fused to form a single guide (sg) nucleic acid.

23. The method of claim 21 or claim 22, wherein the recombinant CRISPR array comprises at least one CRISPR spacer-repeat nucleic acid comprising:
(a) a spacer sequence comprising a 5′ end and a 3′ end; and
(b) a Type II CRISPR repeat sequence comprising a 5′ end and a 3′ end or a Type V repeat sequence comprising a 5′ end and a 3′ end,
wherein the spacer sequence is linked at its 3′end to the 5′ end of the repeat.

24. The method of any one of claims 1 to 3, 7 to 9, or 13 to 21, wherein the plastid transit peptide is a transit peptide from ribulose-1,5-bisphosphate carboxylase/oxygenase small subunit (rbcS), chlorophyll a/b binding protein, biotin carboxyl carrier protein, ferredoxin-dependent glutamate synthase 2 and/or protochlorophyllide oxidoreductase A.

25. The method of any one of claims 1, 4, 5 to 7, 10 to 12, or 13 to 21, wherein the plastid localization sequence is an Eggplant Latent Viroid non-coding RNA sequence, an Avsunviroidae family non-coding RNA sequence, an Avocado sunblotch viroid (ASBVd) non-coding RNA sequence, a Peach latent mosaic viroid (PLMVd) non-coding RNA sequence, a Chrysanthemum chlorotic mottle viroid (CChMVd) non-coding RNA sequence, a (eIF4E) eukaryotic initiation factor 4E, and/or any combination thereof.

26. The method of any one of claims 1, 4 to 7, 10 to 21, wherein the plastid modification cassette comprises a first homology arm and a second homology arm, optionally wherein the plastid modification cassette further comprises an intervening synthetic nucleotide sequence up to about 10 kb in size located between the first and second homology arms.

27. The method of any one of claims 1 to 26, further comprising regenerating a plant from said plant cell having a modified plastid genome.

28. A plant produced by the method of claim 27.

29. A seed produced from the plant of claim 28.

30. A crop comprising a plurality of the plants of claim 28 planted together in an agricultural field, a golf course, a residential lawn, a road side, an athletic field, and/or a recreational field.

31. A product produced from the plant of claim 28, the seed of claim 29, or the crop of claim 30.


SEQUENCE LISTING (33 sequences)

SEQ ID NO: 1 - DNA, 68 bp, Moloney Murine Leukemia Virus
gcgccagtcctccgattgactgagtcgcccgggtacccgtgtatccaataaaccctcttgcagttgca

SEQ ID NO: 2 - DNA, 96 bp, Human immunodeficiency virus type 1
ggtctctctggttagaccagatctgagcctgggagctctctggctaactagggaacccactgcttaagcctcaataaagcttgccttgagtgcttc

SEQ ID NO: 3 - DNA, 77 bp, Moloney Murine Leukemia Virus
tccgacttgtggtctcgctgttccttgggagggtctcctctgagtgattgactacccgtcagcgggggtctttcatt

SEQ ID NO: 4 - DNA, 85 bp, Human immunodeficiency virus type 1
aagtagtgtgtgcccgtctgttgtgtgactctggtaactagagatccctcagacccttttagtcagtgtggaaaatctctagcag

SEQ ID NO: 5 - DNA, 18 bp, Moloney Murine Leukemia Virus
tggggggcgttccgagaa

SEQ ID NO: 6 - DNA, 18 bp, Human immunodeficiency virus type 1
tggcgcccgaacagggac

SEQ ID NO: 7 - DNA, 18 bp, Moloney Murine Leukemia Virus
aaaaaggggggaatgaaa

SEQ ID NO: 8 - DNA, 16 bp, Human immunodeficiency virus type 1
aaaagaaaagggggga

SEQ ID NO: 9 - DNA, 442 bp, Moloney Murine Leukemia Virus
gaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggg

SEQ ID NO: 10 - DNA, 455 bp, Human immunodeficiency virus type 1
ctggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataagatagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgacatcgagcttgctacaagggactttccgctggggactttccagggaggcgtggcctgggcgggactggggagtggcgagccctcagatcctgcatataagcagctgctttttgcctgtactg

SEQ ID NO: 11 - protein, 57 aa, Nicotiana tabacum
MASSVLSSAAVATRSNVAQANMVAPFTGLKSAASFPVSRKQNLDITSIASNGGRVQC

SEQ ID NO: 12 - protein, 85 aa, Arabidopsis thaliana
MLRTVSCLASRSSSSLFFRFFRQFPRSYMSLTSSTAALRVPSRNLRRISSPSVAGRRLLLRRGLRIPSAAVRSVNGQFSRLSVRA

SEQ ID NO: 13 - protein, 35 aa, Chlamydomonas reinhardtii
MALVARPVLSARVAASRPRVAARKAVRVSAKYGEN

SEQ ID NO: 14 - protein, 29 aa, Chlamydomonas reinhardtii
MQALSSRVNIAAKPQRAQRLVVRAEEVKA

SEQ ID NO: 15 - protein, 35 aa, Chlamydomonas reinhardtii
MQTLASRPSLRASARVAPRRAPRVAVVTKAALDPQ

SEQ ID NO: 16 - protein, 31 aa, Chlamydomonas reinhardtii
MQALATRPSAIRPTKAARRSSVVVRADGFIG

SEQ ID NO: 17 - protein, 51 aa, Chlamydomonas reinhardtii
MAFALASRKALQVTCKATGKKTAAKAAAPKSSGVEFYGPNRAKWLGPYSEN

SEQ ID NO: 18 - protein, 50 aa, Chlamydomonas reinhardtii
MAAVIAKSSVSAAVARPARSSVRPMAALKPAVKAAPVAAPAQANQMMVWT

SEQ ID NO: 19 - protein, 40 aa, Chlamydomonas reinhardtii
MAAMLASKQGAFMGRSSFAPAPKGVASRGSLQVVAGLKEV

SEQ ID NO: 20 - protein, 55 aa, Arabidopsis thaliana
MASSMLSSATMVASPAQATMVAPFNGLKSSAAFPATRKANNDITSITSNGGRVNC

SEQ ID NO: 21 - protein, 62 aa, Arabidopsis thaliana
MASSSFSVTSPAAAASVYAVTQTSSHFPIQNRSRRVSFRLSAKPKLRFLSKPSRSSYPVVKA

SEQ ID NO: 22 - protein, 34 aa, Zea mays
MALRTLASKKVLSFPFGGAGRPLAAAASARGVTT

SEQ ID NO: 23 - protein, 30 aa, Oryza sativa
MALRTLASRKTLAAAALPLAAAAAARGVTT

SEQ ID NO: 24 - protein, 30 aa, Hevea brasiliensis
MALRSLVTRKNLPSAFKAATGLGQLRGLQT

SEQ ID NO: 25 - protein, 27 aa, Nicotiana attenuata
MALRTLVSRRTLATGLGFRQQLRGLQT

SEQ ID NO: 26 - protein, 56 aa, Solanum tuberosum
MLRVAGRRLSSSAARSSSTFFTRSSFTVTDDSSPARSPSPSLTSSFLDQIRGFSSN

SEQ ID NO: 27 - protein, 70 aa, Zea mays
MLRVAGRRLSSSLSWRPAAAVARGPLAGAGVPDRDDDSARGRSQPRFSIDSPFFVASRGFSSTETVVPRM

SEQ ID NO: 28 - DNA, 330 bp, Eggplant Latent Viroid
ttggcgaaaccccatttcgacctttcggtctcatcaggggtggcacacaccaccctatggggagaggtcgtcctctatctctcctggaaggccggagcaatccaaaagaggtacacccacccatgggtcgggactttaaattcggaggattcgtcctttaaacgttcctccaagagtcccttccccaaacccttactttgtaagtgtggttcggcgaatgtaccgtttcgtcctttcggactcatcagggaaagtacacactttccgacggtgggttcgtcgacacctctccccctcccaggtactatcccctttccaggatttgttccc

SEQ ID NO: 29 - DNA, 372 bp, Columnea latent viroid
cggaactaaactcgtggttcctgtggttcacacctgaccctgcagccatgcaaaagaaaaaagaacgggagggagagcgcaagagcggtctcaggagccccggggcaactcagaccgagcggggtctcgtggtcgagggcgtacgctgttcagacaggagtaatcccagctgaaacagggttttcacccttcctttcttctggtttccttcctctgcttcagcggcctcgcccggaacctcttgaccagcgcaggtgctgacgcgaccggtggcatcaccgagtttgctcaagcctcaacctcctttttctctattctgtagcttggtctccgggcgagggtgtttagcccttggaaccgcagttggttcct

SEQ ID NO: 30 - DNA, 337 bp, Peach latent mosaic viroid
ctcataagtttcgccgtatctcaacggctcatcagtgggctaagcccagacttatgagagattagtcacctctcagcccctccaccttggggtgccctattcgaggcactgcagtctcgatagaaaggctaagcacgtcgcaatgacgtaaggtgggacttttccttctggaaccaagcggttggttccgaggggggtgtgatccaggtaccgccgtagaaactggattacgacgtctacccgggattccaacccggtcccctccagaagtgattctggatgaagagtcgtgcttagcacactgatgagtctctgaaatgagacgaaactcttttga

SEQ ID NO: 31 - DNA, 5647 bp, Artificial
  PTECv1
ttggcgaaaccccatttcgacctttcggtctcatcaggggtggcacacaccaccctatggggagaggtcgtcctctatctctcctggaaggccggagcaatccaaaagaggtacacccacccatgggtcgggactttaaattcggaggattcgtcctttaaacgttcctccaagagtcccttccccaaacccttactttgtaagtgtggttcggcgaatgtaccgtttcgtcctttcggactcatcagggaaagtacacactttccgacggtgggttcgtcgacacctctccccctcccaggtactatcccctttccaggatttgttcccgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttggggggcgttccgagaatggtagggatgacaggatttgggcgcgccgcggccgcaattcaccgccgtatggctgaccggcgattactagcgattccggcttcatgcaggcgagttgcagcctgcaatccgaactgaggacgggtttttggggttagctcaccctcgcgggatcgcgaccctttgtcccggccattgtagcacgtgtgtcgcccagggcataaggggcatgatgacttgacgtcatcctcaccttcctccggcttatcaccggcagtctgttcagggttccaaactcaacgatggcaactaaacacgagggttgcgctcgttgcgggacttaacccaacaccttacggcacgagctgacgacagccatgcaccacctgtgtccgcgttcccgaaggcacccctctctttcaagaggattcgcggcatgtcaagccctggtaaggttcttcgctttgcatcgaattaaaccacatgctccaccgcttgtgcgggcccccgtcaattcctttgagtttcattcttgcgaacgtactccccaggcgggatacttaacgcgttagctacagcactgcacgggtcgatacgcacagcgcctagtatccatcgtttacggctaggactactggggtatctaatcccattcgctcccctagctttcgtctctcagtgtcagtgtcggcccagcagagtgctttcgccgttggtgttctttccgatctctacgcatttcaccgctccaccggaaattccctctgcccctaccgtactccagcttggtagtttccaccgcctgtccagggttgagccctgggatttgacggcggacttaaaaagccacctacagacgctttacgcccaatcattccggataacgcttgcatcctctgtattaccgcggctgctggcacagagttagccgatgcttattccccagataccgtcattgcttcttctccgggaaaagaagttcacgacccgtgggccttctacctccacgcggcattgctccgtcaggctttcgcccattgcggaaaattccccactgctgcctcccgtaggagtctgggccgtgtctcagtcccagtgtggctgatcatcctctcggaccagctactgatcatcgccttggtaagctattgcctcaccaactagctaatcagacgcgagcccctcctcgggcggattcctccttttgctcctcagcctacggggtattagcagccgtttccagctgttgttcccctcccaagggcaggttcttacgcgttactcacccgtccgccactggaaacaccacttcccgtccgacttgcatgtgttaagcatgccgccagcgttcatcctgagccaggatcgaactctccatgagattcatagttgcattacttatagcttccttgttcgtagacaaagcggattcggaattgtctttcattccaaggcataacttgtatccatgcgcttcatattcgcccggagttcgctcccagaaatatagccatccctgccccctcacgtcaatcccacgagcctcttatccattctcattgaacgacggcgggggagctttcgaggcctcgaaatccaactagaaaaactcacattgggcttagggataatcaggctcgaactgatgacttccaccacgtcaaggtgacactctaccgctgagttatatcccttccccgccccatcgagaaatagaactgactaatcctaagtcaaagggtcgagaaactcaacgccactattcttgaacaacttggagccgggccttcttttcgcactattacggatatgaaaataatggtcaaaatcggattcaattgtcgctcccccgccgtcgttcaatgagaatggataagaggctcgtgggattgacgtgagggggcagggatggctatatttctgggagcgaactccgggcgaatacgaagcgcttggatacagttgtagggagggatttatggcagaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcttgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataagatcctggcctagtctataggaggttttgaaaagaaaggagcaataatcattttcttgttctatcaagagggtgctattgctcctttctttttttctttttatttatttactagtattttacttacatagacttttttgtttacattatagaaaaagaaggagaggttattttcttgcatttattcatgaactgcccctatcggaaataggattgactaccgattccgaaggaactggagttacatctcttttccattcaagagttcttatgcgtttccacgcccctttgagaccccgaaaaatggacaaattccttttcttaggaacacatacaagattcgtcactacaaaaaggataatggtaaccctaccattaactacttcatttatgaatttcatagtaatagaaatacatgtcctaccgagacagaatttggaacttgctatcctcttgcctagcaggcaaagatttacctccgtggaaaggatgattcattcggatcgacatgagagtccaactacattgccagaatccatgttgtatatttgaaagaggttgacctccttgcttctctcatggtacactcctcttcccgccgagccccttttctcctcggtccacagagacaaaatgtaggactggtgccaacaattcatcagactcactaagtcgggatcactaactaatactaatctaatataatagtctaatatatctaatataatagaaaatactaatataatagaaaagaactgtcttttctgtatactttccccggttccgttgctaccgcgggctttacgcaatcgatcggattagatagatatcccttcaacataggtcatcgaaaggatctcggagacccaccaaagtacgaaagccaggatctttcagaaaacggattcctattcaaagagtgcataaccgcatggataagctcacactaacccgtcaatttgggatccaaattcgagattttccttgggaggtatcgggaaggatttggaatggaataatatcgattcatacagaagaaaaggttctctattgattcaaacactgtacctaacctatgggatagggatcgaggaaggggaaaaaccgaagatttcacatggtacttttatcaatctgatttatttcgtacctttcgttcaatgagaaaatgggtcaaattctacaggatcaaacctatgggacttaaggaatgatataaaaaaaagagagggaaaatattcatattaaataaatatgaagtagaagaacccagattccaaatgaacaaattcaaacttgaaaaggatcttccttattcttgaagaatgaggggcaaagggattgatcaagaaagatcgcggccgcagatctaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcgggtatataaggaagttcatttcatttggagagaacacggctgcaggaaagacggttctagaatccgcttaagacctcctaggtccaacgcgttttctactagttacattgagagagcgagacgtcccggtgtgtgtgagagagcagaatcgccggccgccaccttcggcgcagcgccttggcgaaaccccatttcgacctttcggtctcatcaggggtggcacacaccaccctatggggagaggtcgtcctctatctctcctggaaggccggagcaatccaaaagaggtacacccacccatgggtcgggactttaaattcggaggattcgtcctttaaacgttcctccaagagtcccttccccaaacccttactttgtaagtgtggttcggcgaatgtaccgtttcgtcctttcggactcatcagggaaagtacacactttccgacggtgggttcgtcgacacctctccccctcccaggtactatcccctttccaggatttgttcccgcgccagtcctccgattgactgagtcgcccgggtacccgtgtatccaataaaccctcttgcagttgca

SEQ ID NO: 32 - DNA, 5946 bp, Artificial
  PTECv4
ttggcgaaaccccatttcgacctttcggtctcatcaggggtggcacacaccaccctatggggagaggtcgtcctctatctctcctggaaggccggagcaatccaaaagaggtacacccacccatgggtcgggactttaaattcggaggattcgtcctttaaacgttcctccaagagtcccttccccaaacccttactttgtaagtgtggttcggcgaatgtaccgtttcgtcctttcggactcatcagggaaagtacacactttccgacggtgggttcgtcgacacctctccccctcccaggtactatcccctttccaggatttgttcccgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttggggggcgttccgagaatggtagggatgacaggatttgggcgcgccgcggccgcatgtcaccacaaacagagactaaagcaagtgttgggttcaaagctggtgttaaagagtataaattgacttactatactcctgaatatgaaaccaaggatactgatatcttggcagcattccgagtaactcctcaacctggagttccacctgaagaagcaggggctgcggtagctgctgaatcttctactggtacatggacaactgtgtggaccgatgggcttaccagccttgatcgttacaaaggacgatgctaccacatcgagcccgttccaggagaagaaactcaatttattgcgtatgtagcttatcccttagacctttttgaagaaggttcggttactaacatgtttacctcgattgtgggtaatgtatttgggttcaaagccctggctgctctacgtctagaggatctgcgaatccctcctgcttatactaaaactttccaaggaccacctcatggtatccaagttgaaagagataaattgaacaagtatggacgtcccctattaggatgtactattaaaccaaaattggggttatccgcgaaaaactatggtagagcagtttatgaatgtctacgtggtggacttgattttaccaaagatgatgagaatgtgaactcccaaccatttatgcgttggagagaccgtttcttattttgtgccgaagctatttataaatcacaggctgaaacaggtgaaatcaaagggcattatttgaatgctactgcgggtacatgcgaagaaatgatcaaaagagctgtatttgccagagaattgggagttcctatcgtaatgcatgactacttaacagggggattcaccgcaaatactagtttgtctcattattgccgagataatggcctacttcttcacatccaccgtgcaatgcacgctgttattgatagacagaagaatcatggtatgcacttccgtgtactagctaaagctttacgtctatctggtggagatcatattcacgcgggtacagtagtaggtaaacttgaaggagacagggagtcaactttgggctttgttgatttactgcgcgatgattatgttgaaaaagatcgaagccgcggtatctttttcactcaagattgggtctcactacctggtgttctgcctgtggcttcagggggtattcacgtttggcatatgcctgctttgaccgagatctttggagatgattctgtactacaattcggtggaggaactttaggccacccttggggaaatgcaccgggtgccgtagccaaccgagtagctctggaagcatgtgtacaagctcgtaatgagggacgtgatcttgcagtcgagggtaatgaaattatccgtgaagcttgcaaatggagtcctgaactagctgctgcttgtgaagtatggaaagagatcacatttaacttcccaaccatcgataaattagatggccaagagtagatgaattagatttagtaattcacgtttgttttattagtttaattgcactcggctcaatcttttttttactaaaaaagattgagccgaggttatctgttgtatatactattttttttgatagatacatacttaaatttagatagaaaaaaaactcttcaataaaaaaaagaagattaaacacaactacaattttgttattgtagtgttgtgtgctcccccgccgtcgttcaatgagaatggataagaggctcgtgggattgacgtgagggggcagggatggctatatttctgggagcgaactccgggcgaatacgaagcgcttggatacagttgtagggagggatttagtactatggcagaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcttgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaagtactgatcctggcctagtctataggaggttttgaaaagaaaggagcaataatcattttcttgttctatcaagagggtgctattgctcctttctttttttctttttatttatttactagtattttacttacatagacttttttgtttacattatagaaaaagaaggagaggttattttcttgcatttattcatgagtgttatattctttcgtgtcagggcttgaaccaagtatccccgcttcttctaccccatcctgcatgttgtccttttcttttcattccgtattggaataaaaaaagtttaggccagaagctctatggaaaaatcgtggttcaattttatgttttctaagggagaattggaatacagaggtgagctaagtaaagcaatggatagttttgctcctggtgaaaagactactataagtcaagaccgttttatatatgatatggataaaaacttttatggttgggatgagcgttctagttattcttctagttattccaataatgttgatcttttagttagctccaaggacattcgcaatttcatatcggatgacaccttttttgttagggatagtaataagaatagttattctatattttttgataaaaaaaaaaaaatttttgagattgacaatgattttagtgacctagaaaaatttttttatagttattgtagttctagttatctaaataatagatctaaaggtgacaacgatctgcactatgatccttacattaaggatactaaatataattgtactaatcacattaatagttgcattgattcttattttcgttcttacatctgtattgataataactttttaatcgatagtaataattttaatgaaagttacatttataatttcatttgtagtgaaagcggaaagattcgtgaaagtaaaaattacaagataagaactaataggaatcgtagtaatttaataagttctaaggatttcgatataactcaaaactacaatcaattgtggattcaatgcgacaattgttatggattaatgtataagaaagtcaaaatgaatgtttgtgaacaatgtggacattatttgaaaatgagtagttcagaaagaatcgagctttcgattgatccgggtacttggaatcctatggatgaagacatggtctctgcggatcccattaaatttcattcgaaggaggaaccttataaaaaccgtattgactctgcgcaaaaaactacaggattgactgacgctgttcaaacaggtacaggtcaactaaacggtattccggtagcccttggggttatggattttcggtttatggggggtagtatgggatccgtagtaggcgaaaaaataactcgtttgatcgagtatgctaccaatcaatgtttacctcttattttagtgtgttcttccggaggagcacgaatgcaagaaggaagtttaagtttgatgcaaatggctaaaatttcttcggttttatgtgattatcaatcaagtaaaaagttattctatatatcaattcttacatctcctactaccggtggagtgacagctagttttggtatgttgggggatatcattattgccgaaccctatgcctatattgcatttgcgggtaaaagagtaattgaacaaacattgaaaaaagccgtgcctgaaggttcacaagcggctgaatctttattacgtaagggcttattggatgcaattgtaccacgtaatcttttaaaaggtgttctgagcgagttatttcagctccatgctttttttcctttgaacacaaattaagcggccgcctcgagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcgggtatataaggaagttcatttcatttggagagaacacggctgcaggaaagacggttctagaatccgcttaagacctcctaggtccaacgcgttttctactagttacattgagagagcgagacgtcccggtgtgtgtgagagagcagaatcgccggccgccaccttcggcgcagcgccttggcgaaaccccatttcgacctttcggtctcatcaggggtggcacacaccaccctatggggagaggtcgtcctctatctctcctggaaggccggagcaatccaaaagaggtacacccacccatgggtcgggactttaaattcggaggattcgtcctttaaacgttcctccaagagtcccttccccaaacccttactttgtaagtgtggttcggcgaatgtaccgtttcgtcctttcggactcatcagggaaagtacacactttccgacggtgggttcgtcgacacctctccccctcccaggtactatcccctttccaggatttgttcccgcgccagtcctccgattgactgagtcgcccgggtacccgtgtatccaataaaccctcttgcagttgca

SEQ ID NO: 33 - DNA, 5884 bp, Artificial
  PTECv5
ttggcgaaaccccatttcgacctttcggtctcatcaggggtggcacacaccaccctatggggagaggtcgtcctctatctctcctggaaggccggagcaatccaaaagaggtacacccacccatgggtcgggactttaaattcggaggattcgtcctttaaacgttcctccaagagtcccttccccaaacccttactttgtaagtgtggttcggcgaatgtaccgtttcgtcctttcggactcatcagggaaagtacacactttccgacggtgggttcgtcgacacctctccccctcccaggtactatcccctttccaggatttgttcccgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttggggggcgttccgagaatggtagggatgacaggatttgggcgcgccgcggccgcagatctttctcaatcaatctctttgcccctcattcttcgagaatcagaaagagactttttcaagtttgaatttgttcatttggaatctgggttcttctacttcattttgatttacttattttttctttattttctctctcttttctttatttgatttcttttttgattttattcccttccatcattcttaagtcccataagtttgatcctatagaatctgacccattttctcattgagcgaaggggtacgaaataaattcaatcagatttatttttgatcaaaaaaaaaaaaaaaaatcactatgtgaaatcttcgtttttttttttctctttctctatcgctttcccataagtacagcacttgttgaatcgatagagaaccttttcttctgtatcgatatgaatccattatgaatcgatattattacattccaattccttaccaatatccctcaaggaaaatcccgaattggatcccaaattgacgggttagtgtgagcttatccatgcggttatgcactcttcgaataggaattcattttctgaaagattctggctttcgtgctttggcgggtctccgagatcctttcgacgacctatgttgtgttgaagggatatctagatgatccgatcaattgcgtaaagcccgcagtagcaacggaaccggggaaagtatacataagtatacagaaaagacagttcttttcttttctattatattaggattttctattctattagattagttagtgatcttggcgcagtgagtcctttcttccgtgattaactgttggcaccagtcctacattttgtctctgcggaccgagaagaaaggaggctccgcgggaagaggattgtaccatagaagcacggaggtaaacctctttccaatatataaattctggcaatgtagttgggctttcatgttgatccgaatgaatcatctttttcgcggagtgaaatctttgcctgctaggcaagatgataggatagcaagttacaaattctgtttcggtaggacatgtatttctattactatgaaattcataaataaaatagttaatcgtggggttaccattctctctttttttttttcgttatctcgcatgtggtcctaagaaaagggaatttgtcgatttttcggggtcttaaaggggcgtggaaacacataagaactcttgaatggaaatggaaaagagatgtaactccagttcctttggaaataggaagatctttggcgcaagaataaaggattaatccgtatcatcttgacttggttcttatttctctatttttttaagtttaagaaaagaataccgtttctcctacccgtatcgaatagaacatgccgagtcaaatcttcttcatgtaaaaccggcttgatttagatcgggagaatcgtacggttttatgaaaccatgtgctatggctcgaatccgtagtcaatcctatttccgataggagtagttgctcccccgccgtcgttcaatgagaatggataagaggctcgtgggattgacgtgagggggcagggatggctatatttctgggagcgaactccgggcgaatacgaagcgcttggatacagttgtagggagggatttatgcatatggcagaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcttgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaatgcatgatcctggcctagtctataggaggttttgaaaagaaaggagcaataatcattttcttgttctatcaagagggtgctattgctcctttctttttttctttttatttatttactagtattttacttacatagacttttttgtttacattatagaaaaagaaggagaggttattttcttgcatttattcatgcaagttgttcaagaatagtggccttgagtttctcgaccctttgacttaggattagtcagttctatttcttgatgggggaagggatataactcagcggtagagtgtcaccttgacgtggtggaagtcatcagttcgagcctgattatccctaaacccaatgaatgtgagtttttctattttgacttgctccctcgctgtgatcgaataagaatggataagaggctcgtgggattgacgtgagggggtaggggtagctatatttctgggagcgaactccatgcgaatatgaagcgcatggatacaagttatgacttggaatgaaagacaattccgaatcagctttgtctacgaagaaggaagctataagtaatgcaactatgaatctcatggagagttcgatcctggctcaggatgaacgctggcggcatgcttaacacatgcaagtcggacgggaagtggtgtttccagtggcggacgggtgagtaacgcgtaagaacctgcccttgggaggggaacaacagctggaaacggctgctaataccccgtaggctgaggagcaaaaggaggaatccgcccgaggaggggctcgcgtctgattagctagttggtgaggcaatagcttaccaaggcgatgatcagtagctggtccgagaggatgatcagccacactgggactgagacacggcccagactcctacgggaggcagcagtggggaattttccgcaatgggcgaaagcctgacggagcaatgccgcgtggaggtagaaggcctacgggtcctgaacttcttttcccagagaagaagcaatgacggtatctggggaataagcatcggctaactctgtgccagcagccgcggtaatacagaggatgcaagcgttatccggaatgattgggcgtaaagcgtctgtaggtggctttttaagtccgccgtcaaatcccagggctcaaccctggacaggcggtggaaactaccaagcttgagtacggtaggggcagagggaatttccggtggagcggtgaaatgcgtagagatcggaaagaacaccaacggcgaaagcactctgctgggccgacactgacactgagagacgaaagctaggggagcgaatgggattagataccccagtagtcctagccgtaaacgatggatactaggcgctgtgcgtatcgacccgtgcagtgctgtagctaacgcgttaagtatcccgcctggggagtacgttcgcaagaatgaaactcaaaggaattgacgggggcccgcacaagcggtggagcatgtggtttaattcgatgcaaagcgaagaaccttaccagggcttgacatgccgcgaatcctcttgaaagagaggggtgccttcgggaacgcggacacaggtggtgcatggctgtcgtcagctcgtgccgtaaggtgttgggttaagtcccgcaacgagcgcaaccctcgtgtttagttgccaccgttgagtttggaaccctgaacagactgccggtgataagccggaggaaggtgaggatgacgtcaagtcatcatgccccttatgccctgggcgacacacgtgctacaatggccgggacaaagggtcgcgatcccgcgagggtgagctaactccaaaaacccgtcctcagttcggattgcaggctgcaactcgcctgcatgaagccggaatcgctagtaatcgccggtcagccatacggcggtgaattgcggccgccctgcaggaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcgggtatataaggaagttcatttcatttggagagaacacggctgcaggaaagacggttctagaatccgcttaagacctcctaggtccaacgcgttttctactagttacattgagagagcgagacgtcccggtgtgtgtgagagagcagaatcgccggccgccaccttcggcgcagcgccttggcgaaaccccatttcgacctttcggtctcatcaggggtggcacacaccaccctatggggagaggtcgtcctctatctctcctggaaggccggagcaatccaaaagaggtacacccacccatgggtcgggactttaaattcggaggattcgtcctttaaacgttcctccaagagtcccttccccaaacccttactttgtaagtgtggttcggcgaatgtaccgtttcgtcctttcggactcatcagggaaagtacacactttccgacggtgggttcgtcgacacctctccccctcccaggtactatcccctttccaggatttgttcccgcgccagtcctccgattgactgagtcgcccgggtacccgtgtatccaataaaccctcttgcagttgca